(19) United States
(12) Patent Application Publication
(10) Pub. No.: US 2024/0152049 A1
(43) Pub. Date: May 9, 2024

(54) ACTINIC RAY-SENSITIVE OR RADIATION-SENSITIVE RESIN COMPOSITION, RESIST FILM, PATTERN FORMING METHOD, AND METHOD FOR MANUFACTURING ELECTRONIC DEVICE

(71) Applicant: FUJIFILM Corporation, Tokyo (JP)

(72) Inventors: Masafumi KOJIMA, Haibara-gun (JP); Yosuke BEKKI, Hibara-gun (JP); Nobuhiro HIURA, Haibara-gun (JP)

(73) Assignee: FUJIFILM Corporation, Tokyo (JP)

(21) Appl. No.: 18/392,802

(22) Filed: Dec. 21, 2023

Related U.S. Application Data
(63) Continuation of application No. PCT/JP22/21808, filed on May 27, 2022.

(30) Foreign Application Priority Data
Jun. 22, 2021 (JP) 2021-103377

(51) Int. Cl. G03F 7/004; G03F 7/20; G03F 7/32

(52) U.S. Cl. CPC G03F 7/0045; G03F 7/2004; G03F 7/325

(57) ABSTRACT

Provided is an actinic ray-sensitive or radiation-sensitive resin composition that can provide a pattern having good cross-sectional rectangularity. The actinic ray-sensitive or radiation-sensitive resin composition includes: a resin having a group that is decomposed by the action of an acid to generate a polar group; an onium salt (I) that generates an acid represented by formula (1) upon irradiation with actinic rays or radiation; and an onium salt (II) having at least one structural moiety W that includes an anionic moiety A and a cationic moiety M and forms an acidic moiety represented by HA upon irradiation with the actinic rays or radiation. The acid dissociation constant derived from the acidic moiety represented by HA and formed by replacing the cationic moiety in the structural moiety W with H⁺ is larger than the acid dissociation constant of the acid represented by formula (1).

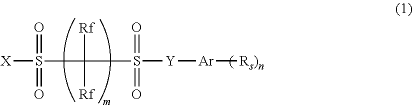

### CROSS-REFERENCE TO RELATED APPLICATIONS

This application is a Continuation of PCT International Application No. PCT/JP2022/021808 filed on May 27, 2022, which claims priority under 35 U.S.C. § 119(a) to Japanese Patent Application No. 2021-103377 filed on Jun. 22, 2021. The above applications are hereby expressly incorporated by reference, in their entirety, into the present application.

### BACKGROUND OF THE INVENTION

### 1. Field of the Invention

The present invention relates to an actinic ray-sensitive or radiation-sensitive resin composition, a resist film, a pattern forming method, and a method for manufacturing an electronic device.

### 2. Description of the Related Art

Examples of the pattern forming method include the following method.

An actinic ray-sensitive or radiation-sensitive resin film formed using an actinic ray-sensitive or radiation-sensitive resin composition (this film is hereinafter referred to as a “resist film”) is exposed to light, so that the solubility of the resin film in a developer is changed in regions in which the exposure pattern is reflected. Then the developer (such as an alkaline aqueous-based or organic solvent-based developer) is used to develop the resist film to remove the exposed or unexposed portions of the resist film, and a desired pattern is thereby obtained.

For example, JP2020-183375A discloses a resist composition including a quencher represented by the following chemical formula and a photoacid generator.

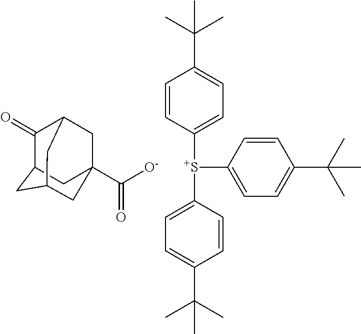 [SMILES: O=C1C2CC3CC1CC(C(=O)[O-])(C3)C2 ; CC(C)(C)c1ccc([S+](c2ccc(C(C)(C)C)cc2)c2ccc(C(C)(C)C)cc2)cc1]

### SUMMARY OF THE INVENTION

The present inventors have conducted studies on the resist composition (actinic ray-sensitive or radiation-sensitive resin composition) described in JP2020-183375A. Then the inventors have found that there is room for improvement in the rectangularity of a cross section of a pattern obtained by exposing to light a resist film formed using the quencher and the photoacid generator disclosed in JP2020-183375A and developing the resist film.

Accordingly, it is an object of the invention to provide an actinic ray-sensitive or radiation-sensitive resin composition that can provide a pattern having good cross-sectional rectangularity.

It is another object of the invention to provide a resist film, a pattern forming method, and a method for manufacturing an electronic device.

The inventors have conducted extensive studies and thus completed the invention. Specifically, the inventors have found that the above objects can be achieved by the following.

[1] An actinic ray-sensitive or radiation-sensitive resin composition including: a resin having a group that is decomposed by the action of an acid to generate a polar group;
an onium salt (I) that generates an acid represented by formula (1) described later upon irradiation with actinic rays or radiation; and
an onium salt (II) having at least one structural moiety W that includes an anionic moiety A and a cationic moiety M and forms an acidic moiety represented by HA upon irradiation with the actinic rays or radiation,
wherein an acid dissociation constant derived from the acidic moiety that is represented by HA and that is formed by replacing the cationic moiety in the structural moiety W with H⁺ is larger than an acid dissociation constant of the acid represented by formula (1).

[2] The actinic ray-sensitive or radiation-sensitive resin composition according to [1], wherein the anionic moiety A in the structural moiety W is a moiety represented by any of formulas (II)-1 to (II)-6 described later.

[3] The actinic ray-sensitive or radiation-sensitive resin composition according to [2], wherein the anionic moiety A in the structural moiety W is the moiety represented by any of formulas (II)-1 and (II)-3 to (II)-6 described later.

[4] The actinic ray-sensitive or radiation-sensitive resin composition according to [2], wherein the anionic moiety A in the structural moiety W is the moiety represented by formula (II)-1 described later.

[5] The actinic ray-sensitive or radiation-sensitive resin composition according to any one of [1] to [4], wherein n in formula (1) is 2 to 5.

[6] The actinic ray-sensitive or radiation-sensitive resin composition according to any one of [1] to [5], wherein R_{S} or R_{S}'s in formula (1) each independently represent —CO—OR_{S1}, —O—CO—R_{S1}, —O—CO—O—R_{S1}, —SO₂—R_{S1}, or —SO₃—R_{S1}, and R_{S1} represents a monovalent substituent.

[7] The actinic ray-sensitive or radiation-sensitive resin composition according to any one of [1] to [6], wherein R_{S} or R_{S}'s in formula (1) each independently represent —CO—OR_{S1} or —O—CO—R_{S1}, and R_{S1} represents a monovalent substituent.

[8] A resist film formed using the actinic ray-sensitive or radiation-sensitive resin composition according to any one of [1] to [7].

[9] A pattern forming method including:
a resist film forming step of forming a resist film on a substrate using the actinic ray-sensitive or radiation-sensitive resin composition according to any one of [1] to [7];
an exposure step of exposing the resist film to light; and
a developing step of developing the exposed resist film using a developer.

[10] The pattern forming method according to [9], wherein, in the exposure step, EUV light is used for exposure.

[11] The pattern forming method according to [9] or [10], wherein the developer includes an organic solvent.

[12] The pattern forming method according to [11], wherein the organic solvent includes butyl acetate.

[13] A method for manufacturing an electronic device, the method including the pattern forming method according to any one of [9] to [12].

The present invention can provide an actinic ray-sensitive or radiation-sensitive resin composition that can provide a pattern having good cross-sectional rectangularity.

The invention can also provide a resist film, a pattern forming method, and a method for manufacturing an electronic device that are related to the actinic ray-sensitive or radiation-sensitive resin composition.

### DESCRIPTION OF THE PREFERRED EMBODIMENTS

The present invention will next be described in detail.

Structural requirements described below may be described on the basis of representative embodiments of the present invention. However, the invention is not limited to these embodiments.

The meanings of notations in the present specification will be described.

In the present specification, a numerical range represented using “to” means a range including the numerical values before and after the “to” as the minimum value and the maximum value, respectively.

In the present specification, the “cross-sectional rectangularity of a pattern” is evaluated as follows. A line pattern having an average line width of 20 nm is formed using a resist film formed on a substrate, and the shape of a cross section of the pattern is observed under a critical dimension scanning electron microscope. The cross-sectional rectangularity is evaluated as the ratio of the pattern line width La in an upper portion of the pattern (a pattern surface on an opposite side from the substrate) to the pattern line width Lb in a bottom portion of the pattern (a pattern surface on the side facing the substrate). Detailed measurement conditions will be described later.

An “organic group” is a group including at least one carbon atom.

As for notations of groups (atomic groups), a notation that is not specified as substituted and unsubstituted is intended to encompass groups having no substituent and groups having a substituent, so long as the notation does not depart from the spirit of the invention. For example, an “alkyl group” is intended to encompass not only an alkyl group having no substituent (an unsubstituted alkyl group) but also an alkyl group having a substituent (a substituted alkyl group).

An “alkyl group” represents a linear or branched alkyl group. A “cycloalkyl group” represents a cyclic alkyl group.

A substituent is a monovalent substituent, unless otherwise specified.

Examples of the substituent include: halogen atoms such as a fluorine atom, a chlorine atom, a bromine atom, and an iodine atom; alkoxy groups such as a methoxy group, an ethoxy group, and a tert-butoxy group; aryloxy groups such as a phenoxy group and a p-tolyloxy group; alkoxycarbonyl groups such as a methoxycarbonyl group, a butoxycarbonyl group, and a phenoxycarbonyl group; acyloxy groups such as an acetoxy group, a propionyloxy group, and a benzoyloxy group; acyl groups such as an acetyl group, a benzoyl group, an isobutyryl group, an acryloyl group, a methacryloyl group, and a methoxalyl group; alkylsulfanyl groups such as a methylsulfanyl group and a tert-butylsulfanyl group; arylsulfanyl groups such as a phenylsulfanyl group and a p-tolylsulfanyl group; alkyl groups; cycloalkyl groups; aryl groups; heteroaryl groups; a hydroxy group; a carboxy group; a formyl group; a sulfo group; a cyano group; alkylaminocarbonyl groups; arylaminocarbonyl groups; sulfonamido groups; silyl groups; an amino group; monoalkylamino groups; dialkylamino groups; arylamino groups; alkylthio groups; and combinations thereof. In the present specification, these substituents are referred to as “substituents K.”

The “actinic rays” or “radiation” means, for example, an emission line spectrum of a mercury lamp, far-ultraviolet rays typified by excimer laser light, extreme ultraviolet light (EUV light), X-rays, an electron beam (EB), etc.

“Light” means actinic rays or radiation.

“Exposure to light” is intended to encompass not only exposure to an emission line spectrum of a mercury lamp, far-ultraviolet rays typified by excimer laser light, X-rays, EUV light, etc. but also image drawing using an electron beam or a particle beam such as an ion beam, unless otherwise specified.

“To” is used to mean that numerical values before and after the “to” are used as the lower limit and the upper limit, respectively.

No limitation is imposed on the bonding direction of a divalent group in the present specification, unless otherwise specified. For example, when Y in a compound represented by a general formula “X—Y—Z” is —COO—, Y may be —CO—O— or may be —O—CO—. This compound may be “X—CO—O—Z” or may be “X—O—CO—Z.”

(Meth)acrylate is intended to refer to both acrylate and methacrylate.

(Meth)acrylic is intended to refer to both acrylic and methacrylic.

The mass average molecular weight (Mw), number average molecular weight (Mn), and dispersity (hereinafter may be referred to also as the “molecular weight distribution”) (Mw/Mn) of a compound are defined as polystyrene-equivalent values determined by GPC (Gel Permeation Chromatography) measurement (solvent: tetrahydrofuran, flow rate (injection amount of a sample): 10 μL, columns: TSK gel Multipore HXL-M manufactured by TOSOH Corporation, column temperature: 40° C., flow velocity: 1.0 mL/minute, detector: differential refractive index detector) using a GPC apparatus (HLC-8120GPC manufactured by TOSOH Corporation).

The compositional ratio (molar ratio, mass ratio, etc.) of a resin is measured by ¹³C-NMR (nuclear magnetic resonance).

The acid dissociation constant (pKa) is the pKa in an aqueous solution and is specifically a value determined by computation using the following software package 1 based on a Hammett substituent constant and a database of known literature values. All pKa values in the present specification are values determined by computation using this software package. Software package 1: Advanced Chemistry Development (ACD/Labs) Software V 8.14 for Solaris (1994-2007 ACD/Labs).

The pKa can also be determined by a molecular orbital calculation method. In one specific example of this method, H⁺ dissociation free energy in an aqueous solution is computed based on a thermodynamic cycle to compute the pKa. As for the method for computing the H⁺ dissociation free energy, the density functional theory (DFT), for example, can be used for the computation. Various other methods have been reported in literature etc., but the computation method is not limited thereto. There are a plurality of software applications capable of performing the DFT, and one example is Gaussian 16.

The pKa is a value determined by computation using the software package 1 based on the Hammett substituent constant and the database of known literature values as described above. When the pKa cannot be computed using this method, a value obtained using Gaussian 16 based on the DFT (density functional theory) is used.

As described above, the pKa means the “pKa in an aqueous solution.” However, when the pKa in an aqueous solution cannot be computed, the “pKa in a dimethyl sulfoxide (DMSO) solution” is used.

“Solids” are components forming a resist film, and a solvent is not included. Any component included in the film is considered as a solid component even when the component is in a liquid form.

“1 inch” means 25.4 mm.

### <Actinic Ray-Sensitive or Radiation-Sensitive Resin Composition>

The actinic ray-sensitive or radiation-sensitive resin composition of the invention includes: a resin having a group that is decomposed by the action of an acid to generate a polar group; an onium salt (I) that generates an acid represented by formula (1) below upon irradiation with actinic rays or radiation; and an onium salt (II) having at least one structural moiety W that includes an anionic moiety A and a cationic moiety M and forms an acidic moiety represented by HA upon irradiation with the actinic rays or radiation. The acid dissociation constant derived from the acidic moiety represented by HA and formed by replacing the cationic moiety in the structural moiety W with H⁺ is larger than the acid dissociation constant of the acid represented by formula (1).

The “actinic ray-sensitive or radiation-sensitive resin composition” is hereinafter referred to also as a “resist composition.”

The “resin having the group that is decomposed by the action of an acid to generate a polar group” is hereinafter referred to as an “acid decomposable resin.”

The “acid dissociation constant derived from the acidic moiety represented by HA and formed by replacing the cationic moiety in the structural moiety W with H⁺” is referred to also as the “acid dissociation constant of HA in the structural moiety W.”

When the structure described above is used, a pattern obtained by exposing the resist film to light and developing the resist film has good cross-sectional rectangularity. The mechanism for this is unclear. However, the inventors infer that the mechanism is as follows.

The onium salt (I) interacts strongly with the acid decomposable resin included in the resist composition, and diffusion of the acid generated in the exposed portions of the resist film to the unexposed portions is reduced. Moreover, the acid dissociation constant of the acidic moiety in the structural moiety W in the onium salt (II) included in the resist composition of the invention is larger than the acid dissociation constant of the acid generated from the onium salt (I). Therefore, the onium salt (II) in the unexposed portions of the resist film can function as a quencher for the acid generated from the onium salt (I). Thus, the diffusion of the acid generated from the onium salt (I) to the unexposed portions is further reduced by the action of the onium salt (II) in the unexposed portions. As a result of this, the acid generated in the exposed portions of the resist film when the resist film formed on the substrate is exposed to light and developed to obtain a pattern does not diffuse. In this case, the width of the pattern removed during development on a surface of the resist film on the side facing the substrate is substantially the same as that on a surface on the opposite side from the substrate, and the pattern has good cross-sectional rectangularity.

When the pattern obtained has further improved cross-sectional rectangularity, it is said that “the effects of the invention are further enhanced.”

The resist composition of the invention will next be described in detail.

The resist composition may be a positive-type resist composition or a negative-type resist composition. Moreover, the resist composition of the invention may be a resist composition for alkali development or may be a resist composition for organic solvent development.

The resist composition may by a non-chemical amplification resist composition. The resist composition may also have the function as a chemical amplification resist composition.

The components of the resist composition will be described in detail.

### [Onium Salt (I)]

The onium salt (I) is an onium salt that generates an acid represented by formula (1) described later upon irradiation with actinic rays or radiation. Therefore, the onium salt (I) functions as a photoacid generator.

The onium salt is a compound having an anionic moiety and a cationic moiety in its molecule. In the onium salt (I), the anionic moiety is an anion derived from the acid represented by formula (1) below.

The acid represented by formula (1) and the cationic moiety in the onium salt (I) will be described.

### (Acid Represented by Formula (1))

Formula (1) is shown below.

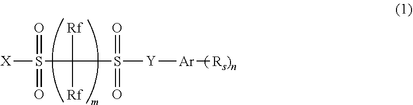

In formula (1), X represents —OH or —NH—SO₂—R_{X}. R_{X} represents an alkyl group having at least one fluorine atom.

Rf represents a fluorine atom or an alkyl group having at least one fluorine atom.

Y represents a single bond, an oxygen atom, or a sulfur atom.

Ar represents an n+1 valent aromatic ring group optionally having a substituent other than R_{S}.

R_{S} represents —O—R_{S1}, —CO—R_{S1}, —CO—O—R_{S1}, —O—CO—R_{S1}, —O—CO—O—R_{S1}, —SO₂—R_{S1}, or —SO₃—R_{S1}. R_{S1} represents a monovalent substituent.

m represents an integer of 1 or more.

n represents an integer of 1 to 5.

The requirements of the acid represented by formula (1) will be described in detail.

In formula (1), X represents —OH or —NH—SO₂—R_{X}. R_{X} represents an alkyl group having at least one fluorine atom.

No particular limitation is imposed on the number of carbon atoms in the alkyl group moiety in R_{X}, but the number of carbon atoms is preferably 1 to 6 and more preferably 1 to 2.

The alkyl group has at least one fluorine atom. Specifically, it is only necessary that at least one hydrogen atom in the alkyl group be replaced with a fluorine atom, and all the hydrogen atoms in the alkyl group may be replaced with fluorine atoms. In particular, it is preferable that all the hydrogen atoms in the alkyl group are replaced with fluorine atoms. Specifically, R_{X} is preferably a perfluoroalkyl group.

H in X is easily dissociated from X, and X forms an atomic group that acts as an acid. Therefore, the acid dissociation constant of the acid represented by formula (1) is the dissociation constant of H in X. No particular limitation is imposed on the acid dissociation constant of the acid represented by formula (1) so long as it is smaller than the acid dissociation constant of the structural moiety W of the onium salt (II). The acid dissociation constant of the acid represented by formula (1) is preferably 2.0 or less, more preferably 0.5 or less, and still more preferably −3.0 or less. No particular limitation is imposed on the lower limit of the acid dissociation constant, but the lower limit may be −15.0 or more.

In formula (1), Rf represents a fluorine atom or an alkyl group having at least one fluorine atom.

When Rf is an alkyl group having at least one fluorine atom, no particular limitation is imposed on the number of carbon atoms in the alkyl group moiety in Rf, and the number of carbon atoms is preferably 1 to 6 and more preferably 1 to 2.

The alkyl group has at least one fluorine atom. Specifically, it is only necessary that at least one hydrogen atom in the alkyl group be replaced with a fluorine atom, and all the hydrogen atoms in the alkyl group may be replaced with fluorine atoms. In particular, it is preferable that all the hydrogen atoms in the alkyl group are replaced with fluorine atoms. Specifically, when Rf is an alkyl group having at least one fluorine atom, Rf is preferably a perfluoroalkyl group.

In particular, Rf is preferably a fluorine atom.

In formula (1), Y represents a single bond, an oxygen atom, or a sulfur atom.

Y is preferably a single bond or an oxygen atom and more preferably an oxygen atom.

In formula (1), Ar represents an n+1 valent aromatic ring group. The n+1 valent aromatic ring group corresponds to a group formed by removing n+1 hydrogen atoms from the aromatic ring. When, for example, n+1 is 2, Ar represents a divalent aromatic group (an arylene group or a heteroarylene group).

No particular limitation is imposed on the aromatic ring group, and the aromatic ring included in the aromatic ring group may be a monocyclic ring or a polycyclic ring.

Examples of the aromatic ring included in the aromatic ring group include aromatic hydrocarbon rings and aromatic heterocyclic rings. Examples of the aromatic hydrocarbon ring include a benzene ring, a naphthalene ring, an anthracene ring, a phenanthrene ring, and a pyrene ring. Examples of the aromatic heterocyclic ring include a furan ring, a pyrrole ring, a pyrazole ring, an imidazole ring, a thiophene ring, an oxazole ring, and a thiazole ring.

When the aromatic ring included in the aromatic ring group is a polycyclic ring, the aromatic ring may be a polycyclic ring formed by combining an aromatic hydrocarbon ring and an aromatic heterocyclic ring. Examples of the polycyclic ring include an indole ring, an isoindole ring, a benzimidazole ring, a purine ring, a carbazole ring, a benzofuran ring, an isobenzofuran ring, a benzothiophene ring, a benzoxazole ring, and a benzothiazole ring.

In particular, the aromatic ring group is preferably an aromatic hydrocarbon ring and more preferably a benzene ring.

Ar may have a substituent other than R_{S}. Examples of the substituent other than R_{S} include alkyl groups. The number of carbon atoms in the alkyl group is preferably 1 to 5. Examples of the alkyl group include a methyl group, an ethyl group, a propyl group, an isopropyl group, a sec-butyl group, and a t-butyl group. Of these, a methyl group is preferred.

The alkyl group may have a substituent. Examples of the substituent include the substituents K, and the substituent is preferably a halogen atom.

In formula (1), R_{S} represents —O—R_{S1}, —CO—R_{S1}, —CO—O—R_{S1}, —O—CO—R_{S1}, —O—CO—O—R_{S1}, —SO₂-R_{S1}, or —SO₃-R_{S1}. R_{S1} represents a monovalent substituent.

When n is 2 or more, R_{S}'s may be the same or different. When n is 2 or more, R_{S}'s may be bonded together to form a ring.

In terms of further enhancing the effects of the invention, R_{S} is preferably —CO—R_{S1}, —CO—O—R_{S1}, —O—CO—R_{S1}, —O—CO—O—R_{S1}, —SO₂-R_{S1}, or —SO₃-R_{S1}, more preferably —CO—OR_{S1}, —O—CO—R_{S1}, —O—CO—O—R_{S1}, —SO₂—R_{S1}, or —SO₃—R_{S1}, and still more preferably —CO—O—R_{S1} or —O—CO—R_{S1}.

No particular limitation is imposed on R_{S1} so long as it is a monovalent substituent. Examples of R_{S1} include the substituents K. Specific examples of R_{S1} include alkyl groups optionally having a substituent, cycloalkyl groups optionally having a substituent, aryl groups optionally having a substituent, and combinations thereof. The alkyl, cycloalkyl, and aryl groups optionally having a substituent will be described.

The alkyl group optionally having a substituent in R_{S1} may be linear or branched. No particular limitation is imposed on the number of carbon atoms in the alkyl group optionally having a substituent, and the number of carbon atoms is preferably 1 to 8 and more preferably 1 to 4.

Examples of the alkyl group moiety in the alkyl group optionally having a substituent include a methyl group, an ethyl group, a propyl group, an isopropyl group, a sec-butyl group, and a t-butyl group.

Examples of the optional substituent of the alkyl group include the substituents K. Of these, halogen atoms are preferred, and a fluorine atom is more preferred. Any hydrogen atom in the alkyl group may be replaced with a halogen atom. In this case, some of the hydrogen atoms in the alkyl group may be replaced with halogen atoms, or all the hydrogen atoms may be replaced with halogen atoms.

The cycloalkyl group optionally having a substituent in R_{S1} may be a monocyclic ring or a polycyclic ring. No particular limitation is imposed on the number of carbon atoms in the cycloalkyl group optionally having a substituent, and the number of carbon atoms is preferably 4 to 20 and more preferably 4 to 16.

Examples of the cycloalkyl group moiety in the cycloalkyl group optionally having a substituent include: monocyclic cycloalkyl groups such as a cyclopropyl group, a cyclobutyl group, a cyclopentyl group, a cyclohexyl group, and a cycloheptyl group; and polycyclic cycloalkyl groups such as a norbornyl group, a camphor residue, a tricyclodecanyl group, a tetracyclodecanyl group, a tetracyclododecanyl group, and an adamantyl group.

It is also preferable that —(CH₂)— in the cycloalkyl group is replaced with a divalent substituent. The divalent substituent for —(CH₂)— in the cycloalkyl group is preferably —O—, —S—, —CO—, —OC—O—, or —CO—O— and more preferably —O—. The number of divalent substituents for —(CH₂)— in the cycloalkyl group is preferably 1 to 3 and more preferably 1.

Examples of the optional substituent in the cycloalkyl group include the substituents K, and the substituent is preferably an alkyl group. Preferred modes and specific examples of the alkyl group are the same as those described for the alkyl group moiety in the alkyl group optionally having a substituent.

The aryl group optionally having a substituent in R_{S1} may be a monocyclic ring or a polycyclic ring. The aryl group optionally having a substituent may be a heteroaryl group which optionally has a substituent and in which the atoms forming the ring include an atom other than carbon atoms. No particular limitation is imposed on the number of carbon atoms in the aryl group optionally having a substituent, and the number of carbon atoms is preferably 6 to 20 and more preferably 6 to 10.

Specific examples of the aromatic ring moiety included in the aryl group optionally having a substituent are the same as those for the aromatic ring in Ar. In particular, the aromatic ring moiety included in the aryl group optionally having a substituent is preferably a benzene ring.

Examples of the optional substituent in the aryl group include the substituents K. Of these, an alkyl group optionally having a substituent is preferred. No particular limitation is imposed on the number of carbon atoms in the alkyl group optionally having a substituent, and the number of carbon atoms is preferably 1 to 8 and more preferably 1 to 4.

Specific examples of the alkyl group optionally having a substituent are the same as those for the above-described alkyl group optionally having a substituent, and their preferred modes are also the same as those for the above-described alkyl group.

No particular limitation is imposed on the number of substituents in the aryl group optionally having a substituent. The number of substituents is preferably 1 to 3 and more preferably 1 to 2.

In formula (1), m is an integer of 1 or more.

m is preferably an integer of 1 to 5 and more preferably an integer of 1 to 3.

In formula (1), n represents an integer of 1 to 5.

In terms of further enhancing the effects of the invention, n is preferably an integer of 2 to 5, more preferably an integer of 2 to 3, and still more preferably 2.

Preferably, n is an integer of 2 to 3, and R_{S} is —CO—O—R_{S1} or —O—CO—R_{S1}.

### (Cationic Moiety)

The cationic moiety in the onium salt (I) is a structural moiety including a positively charged atom or atomic group and is, for example, a singly charged organic cation. Preferred modes of the organic cation will next be described.

The organic cationic moiety in the onium salt (I) is preferably an organic cation (cation (Za1)) represented by formula (ZaI) or an organic cation (cation (ZaII)) represented by formula (ZaII).

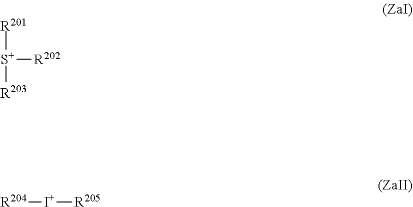

In formula (ZaI), R²⁰¹, R²⁰², and R²⁰³ each independently represent an organic group. The number of carbon atoms in each of the organic groups used as R²⁰¹, R²⁰², and R²⁰³ is generally 1 to 30 and preferably 1 to 20. Two selected from the group consisting of R²⁰¹ to R²⁰³ may be bonded together to form a ring structure, and the ring may include an oxygen atom, a sulfur atom, —CO—O—, an amido group, or a carbonyl group. Examples of the group formed from two selected from the group consisting of R²⁰¹ to R²⁰³ that are bonded together include alkylene groups (such as a butylene group and a pentylene group) and —CH₂—CH₂—O—CH₂—CH₂—.

Preferred examples of the form of the organic cation in formula (ZaI) include a cation (ZaI-1), a cation (ZaI-2), an organic cation (cation (ZaI-3b)) represented by formula (ZaI-3b), and an organic cation (cation (ZaI-4b)) represented by formula (ZaI-4b) that will be described later.

First, the cation (ZaI-1) will be described.

The cation (ZaI-1) is an arylsulfonium cation in which at least one of R²⁰¹, R²⁰², or R²⁰³ in formula (ZaI) is an aryl group.

In the arylsulfonium cation, each of R²⁰¹ to R²⁰³ may be an aryl group. Alternatively, some of R²⁰¹ to R²⁰³ may be an aryl group, and the rest may be an alkyl group or a cycloalkyl group.

Alternatively, one of R²⁰¹, R²⁰², or R²⁰³ may be an aryl group, and the remaining two of R²⁰¹ to R²⁰³ may be bonded together to form a ring structure. The ring may include an oxygen atom, a sulfur atom, —CO—O—, an amido group, or a carbonyl group. Examples of the group formed by bonding two selected from the group consisting of R²⁰¹ to R²⁰³ together include alkylene groups in which at least one methylene group is replaced by an oxygen atom, a sulfur atom, —CO—O—, an amido group, and/or a carbonyl group (such as a butylene group, a pentylene group, and a —CH₂—CH₂—O—CH₂—CH₂—).

Examples of the arylsulfonium cation include triarylsulfonium cations, diarylalkylsulfonium cations, aryldialkylsulfonium cations, diarylcycloalkylsulfonium cations, and aryldicycloalkylsulfonium cations.

Each aryl group included in the arylsulfonium cation is preferably a phenyl group or a naphthyl group and is more preferably a phenyl group. The aryl group may have a heterocyclic structure having an oxygen atom, a nitrogen atom, a sulfur atom, etc. Examples of the heterocyclic structure include a pyrrole residue, a furan residue, a thiophene residue, an indole residue, a benzofuran residue, and a benzothiophene residue. When the arylsulfonium cation has two or more aryl groups, the two or more aryl groups may be the same or different.

The alkyl group or the cycloalkyl group optionally included in the arylsulfonium cation is preferably a linear alkyl group having 1 to 15 carbon atoms, a branched alkyl group having 3 to 15 carbon atoms, or a cycloalkyl group having 3 to 15 carbon atoms and more preferably, for example, a methyl group, an ethyl group, a propyl group, a n-butyl group, a sec-butyl group, a t-butyl group, a cyclopropyl group, a cyclobutyl group, a cyclohexyl group, etc.

The aryl, alkyl, and cycloalkyl groups in R²⁰¹ to R²⁰³ may each independently have a substituent, and the substituent is preferably an alkyl group (having, for example, 1 to 15 carbon atoms), a cycloalkyl group (having, for example, 3 to 15 carbon atoms), an aryl group (having, for example, 6 to 14 carbon atoms), an alkoxy group (having, for example, 1 to 15 carbon atoms), a cycloalkylalkoxy group (having, for example, 1 to 15 carbon atoms), a halogen atom (for example, fluorine or iodine), a hydroxy group, a carboxy group, —CO—O—, a sulfinyl group, a sulfonyl group, an alkylthio group, or a phenylthio group.

Each substituent may have a substituent if possible. It is also preferable that the alkyl group has, for example, a halogen atom as a substituent and is therefore a halogenated alkyl group such as a trifluoromethyl group.

It is also preferable that any of these substituents are combined together to form an acid-decomposable group.

The acid-decomposable group means a group that is decomposed by the action of an acid to generate an acid group and preferably has a structure in which the acid group is protected by a leaving group that leaves by the action of an acid. The details of the acid group and the leaving group will be described later together with the acid decomposable resin.

Next, the cation (ZaI-2) will be described.

The cation (ZaI-2) is a cation in which R²⁰¹ to R²⁰³ in formula (ZaI) each independently represent an organic group having no aromatic ring. The aromatic ring is intended to encompass an aromatic ring including a heteroatom.

The number of carbon atoms in each of the organic groups having no aromatic ring and represented by R²⁰¹ to R²⁰³ is generally 1 to 30 and preferably 1 to 20.

R²⁰¹ to R²⁰³ are each independently preferably an alkyl group, a cycloalkyl group, an allyl group, or a vinyl group, more preferably a linear or branched 2-oxoalkyl group, a 2-oxocycloalkyl group, or an alkoxycarbonylmethyl group, and still more preferably a linear or branched 2-oxoalkyl group.

Examples of the alkyl and cycloalkyl groups in R²⁰¹ to R²⁰³ include: linear alkyl groups having 1 to 10 carbon atoms and branched alkyl groups having 3 to 10 carbon atoms (such as a methyl group, an ethyl group, a propyl group, a butyl group, and a pentyl group); and cycloalkyl groups having 3 to 10 carbon atoms (such as a cyclopentyl group, a cyclohexyl group, and a norbornyl group).

R²⁰¹ to R²⁰³ may each be further substituted with a halogen atom, an alkoxy group (having, for example, 1 to 5 carbon atoms), a hydroxy group, a cyano group, or a nitro group.

It is also preferable that the substituents in R²⁰¹ to R²⁰³ are each independently combined with another substituent to form an acid-decomposable group.

Next, the cation (ZaI-3b) will be described.

The cation (ZaI-3b) is a cation represented by formula (ZaI-3b).

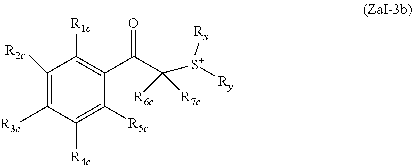

In formula (ZaI-3b), R_{1c} to R_{5c} each independently represent a hydrogen atom, an alkyl group, a cycloalkyl group, an aryl group, an alkoxy group, an aryloxy group, an alkoxycarbonyl group, an alkylcarbonyloxy group, a cycloalkylcarbonyloxy group, a halogen atom, a hydroxy group, a nitro group, an alkylthio group, or an arylthio group.

R_{6c} and R_{7c} each independently represent a hydrogen atom, an alkyl group (such as a t-butyl group), a cycloalkyl group, a halogen atom, a cyano group, or an aryl group.

Rₓ and R_{y} each independently represent an alkyl group, a cycloalkyl group, a 2-oxoalkyl group, a 2-oxocycloalkyl group, an alkoxycarbonylalkyl group, an allyl group, or a vinyl group.

R_{1c} to R_{7c}, Rₓ, and R_{y} may each have a substituent, and it is also preferable that these substituents are each independently combined with another substituent to form an acid-decomposable group.

A combination of two or more selected from the group consisting of R_{1c} to R_{5c}, a pair of R_{5c} and R_{6c}, a pair of R_{6c} and R_{7c}, a pair of R_{5c} and Rₓ, and a pair of Rₓ and R_{y} may each be bonded together to form a ring. These rings may each independently include an oxygen atom, a sulfur atom, a ketone group, an ester bond, or an amide bond.

Each ring may be an aromatic or non-aromatic hydrocarbon ring, an aromatic or non-aromatic heterocyclic ring, or a polycyclic condensed ring formed by combining two or more of the above rings. The ring may be a 3- to 10-membered ring and is preferably a 4- to 8-membered ring and more preferably a 5- or 6-membered ring.

Examples of the groups formed by bonding two or more selected from the group consisting of R_{1c} to R_{5c}, bonding R_{6c} and R_{7c}, and bonding Rₓ and R_{y} include alkylene groups such as a butylene group and a pentylene group. A methylene group in the alkylene group may be replaced with a heteroatom such as an oxygen atom.

The group formed by bonding R_{5c} and R_{6c} and the group formed by bonding R_{5c} and Rₓ are each preferably a single bond or an alkylene group. Examples of the alkylene group include a methylene group and an ethylene group.

R_{1c} to R_{5c}, R_{6c}, R_{7c}, Rₓ, R_{y}, the ring formed by bonding together a combination of two or more selected from the group consisting of R_{1c} to R_{5c}, the ring formed by bonding together a pair of R_{5c} and R_{6c}, the ring formed by bonding together a pair of R_{6c} and R_{7c}, the ring formed by bonding together a pair of R_{5c} and Rₓ, and the ring formed by bonding together a pair of Rₓ and R_{y} may each have a substituent.

Next, the cation (ZaI-4b) will be described.

The cation (ZaI-4b) is a cation represented by formula (ZaI-4b).

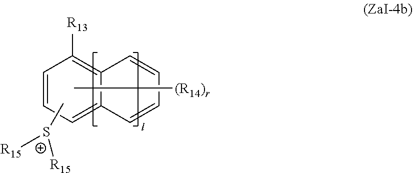

In formula (ZaI-4b), 1 represents an integer of from 0 to 2.

r represents an integer of from 0 to 8.

R₁₃ represents a hydrogen atom, a halogen atom (such as a fluorine atom or an iodine atom), a hydroxy group, an alkyl group, a halogenated alkyl group, an alkoxy group, a carboxy group, an alkoxycarbonyl group, or a group including a cycloalkyl group (a cycloalkyl group itself or a group having a cycloalkyl group as a part thereof). These groups may each further have a substituent.

R₁₄ represents a hydroxy group, a halogen atom (such as a fluorine atom or an iodine atom), an alkyl group, a halogenated alkyl group, an alkoxy group, an alkoxycarbonyl group, an alkylcarbonyl group, an alkylsulfonyl group, a cycloalkylsulfonyl group, or a group including a cycloalkyl group (a cycloalkyl group itself or a group having a cycloalkyl group as a part thereof). These groups may each have a substituent. When a plurality of R₁₄'s are present, they each independently represent any of the above groups such as a hydroxy group.

R₁₅'s each independently represent an alkyl group, a cycloalkyl group, or a naphthyl group. The two R₁₅'s may be bonded together to form a ring. When the two R₁₅'s are bonded together to form a ring, the skeleton of the ring may include a heteroatom such as an oxygen atom or a nitrogen atom.

In one preferred mode, the two R₁₅'s are each an alkylene group and are bonded together to form a ring structure. The above alkyl, cycloalkyl, and naphthyl groups and the ring formed by bonding the two R₁₅'s may each have a substituent.

The alkyl group represented by each of R₁₃, R₁₄, and R₁₅s may be a linear or branched alkyl group. Preferably, the number of carbon atoms in the alkyl group is 1 to 10. Each alkyl group is more preferably a methyl group, an ethyl group, a n-butyl group, a t-butyl group, etc.

It is also preferable that the substituents in R₁₃ to R₁₅'s, Rₓ, and R_{y} are each independently combined with another substituent to form an acid-decomposable group.

Next, formula (ZaII) will be described.

In formula (ZaII), R²⁰⁴ and R²⁰⁵ each independently represent an aryl group, an alkyl group, or a cycloalkyl group.

The aryl group represented by each of R²⁰⁴ and R²⁰⁵ is preferably a phenyl group or a naphthyl group and more preferably a phenyl group. The aryl group represented by each of R²⁰⁴ and R²⁰⁵ may be an aryl group having a heterocycle having an oxygen atom, a nitrogen atom, or a sulfur atom. Examples of the skeleton of the aryl group having a heterocycle include pyrrole, furan, thiophene, indole, benzofuran, and benzothiophene.

The alkyl or cycloalkyl group represented by each of R²⁰⁴ and R²⁰⁵ is preferably a linear alkyl group having 1 to 10 carbon atoms or a branched alkyl group having 3 to 10 carbon atoms (such as a methyl group, an ethyl group, a propyl group, a butyl group, or a pentyl group) or is a cycloalkyl group having 3 to 10 carbon atoms (such as a cyclopentyl group, a cyclohexyl group, or a norbornyl group).

The aryl, alkyl, and cycloalkyl groups represented by R²⁰⁴ and R²⁰⁵ may each independently have a substituent. Examples of the optional substituents in the aryl, alkyl, and cycloalkyl groups represented by R²⁰⁴ and R²⁰⁵ include alkyl groups (having, for example, 1 to 15 carbon atoms), cycloalkyl groups (having, for example, 3 to 15 carbon atoms), aryl groups (having, for example, 6 to 15 carbon atoms), alkoxy groups (having, for example, 1 to 15 carbon atoms), halogen atoms, a hydroxy group, and a phenylthio group. It is also preferable that the substituents in R²⁰⁴ and R²⁰⁵ are each independently combined with another substituent to form an acid-decomposable group.

The molecular weight of the onium salt (I) is preferably 100 to 10000, more preferably 100 to 2500, and still more preferably 100 to 1500.

The content of the onium salt (I) with respect to the total mass of solids in the resist composition is preferably 1% by mass or more, more preferably 2% by mass or more, and still more preferably 4% by mass or more. The upper limit of the content with respect to the total mass of the solids in the resist composition is preferably 70% by mass or less, more preferably 50% by mass or less, and still more preferably 40% by mass or less.

One onium salt (I) may be used alone, or two or more onium salts (I) may be used. When two or more onium salts (I) are used, it is preferable that the total content falls within the above preferred range.

### [Onium Salt (II)]

The onium salt (II) includes at least one structural moiety W that includes an anionic moiety A and a cationic moiety M and forms an acidic moiety represented by HA upon irradiation with actinic rays or radiation, and the acid dissociation constant derived from the acidic moiety represented by HA and formed by replacing the cationic moiety in the structural moiety W with H⁺ (the acid dissociation constant of HA in the structural moiety W) is larger than the acid dissociation constant of the acid represented by formula (1) above.

The onium salt (II) may further have, in addition to the structural moiety W, a structural moiety Y that includes an anionic moiety A_{Y} and a cationic moiety M_{Y} and forms an acidic moiety represented by HA_{Y} upon irradiation with actinic rays or radiation. The acid dissociation constant derived from the acidic moiety represented by HA_{Y} and formed by replacing the cationic moiety M_{Y} in the structural moiety Y with H⁺ (the acid dissociation constant of HA_{Y} in the structural moiety Y) is the same as the acid dissociation constant of the acid represented by formula (1) or smaller than the acid dissociation constant of the acid represented by formula (1).

First, the structural moiety W and the structural moiety Y will be described.

### (Structural Moiety W)

As described above, the structural moiety W includes the anionic moiety A and the cationic moiety M.

The onium salt (II) may have a plurality of the structural moieties W.

The anionic moiety A and the cationic moiety M in the structural moiety W will be described in detail.

### —Anionic Moiety A—

The anionic moiety A is a structural moiety including a negatively charged atom or atomic group. No particular limitation is imposed on the anionic moiety A so long as the acid dissociation constant of HA in the structural moiety W is larger than the acid dissociation constant of the acid represented by formula (1) above. Preferred modes of the anionic moiety A will be described.

The acid dissociation constant of HA in the structural moiety W is, for example, 12.0 or less and preferably 10.0 or less. The lower limit of the acid dissociation constant is preferably −4.0 or more.

The difference between the acid dissociation constant of the acid represented by formula (1) and the acid dissociation constant of HA in the structural moiety W is preferably 1.0 or more and more preferably 1.5 or more.

In terms of further enhancing the effects of the invention, the anionic moiety A in the structural moiety W is preferably a moiety represented by any of formulas (II)-1 to (II)-6.

In formulas (II)-1 to (II)-6, * represents a direct bond.

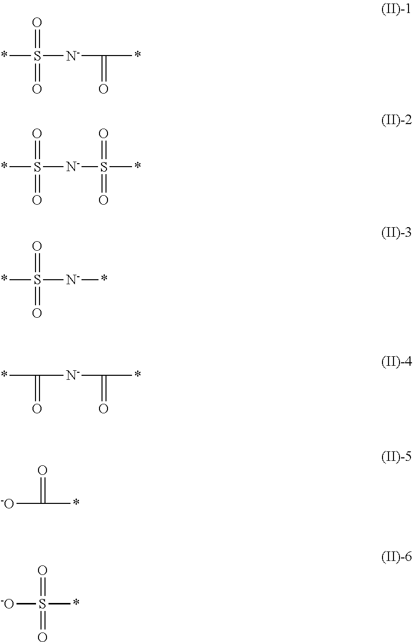

In terms of further enhancing the effects of the invention, the anionic moiety A in the structural moiety W is more preferably a moiety represented by any of formulas (II)-1 and (II)-3 to (II)-6 and more preferably a moiety represented by formula (II)-1.

—Cationic moiety M—

The cationic moiety M is a structural moiety including a positively charged atom or atomic group. No particular limitation is imposed on the cationic moiety M so long as the structural moiety W can form the acidic moiety represented by HA upon irradiation with actinic rays or radiation, and the definition and preferred modes of the cationic moiety M are as described above for the cationic moiety in the onium salt (I).

### (Structural Moiety Y)

As described above, the structural moiety Y includes the anionic moiety A_{Y} and the cationic moiety My.

The onium salt (II) may have a plurality of the structural moieties Y.

The anionic moiety A_{Y} and cationic moiety M_{Y} in the structural moiety Y will be described in detail.

—Anionic moiety A_{Y}—

The anionic moiety A_{Y} is a structural moiety including a negatively charged atom or atomic group. The acid dissociation constant derived from the acidic moiety represented by HA_{Y} and formed by replacing the cationic moiety M_{Y} in the structural moiety Y including the anionic moiety A_{Y} with H⁺ is the same as the acid dissociation constant of the acid represented by formula (1) or smaller than the acid dissociation constant of the acid represented by formula (1).

The anionic moiety A_{Y} will be described.

Examples of the anionic moiety A_{Y} include —SO₃⁻ and —SO₂—NH—SO₂—R_{X}. R_{X} included in the structure exemplified as the anionic moiety A_{Y} is the same as the R_{X} described for formula (1) above.

—Cationic moiety M_{Y}—

The cationic moiety M_{Y} is a structural moiety including a positively charged atom or atomic group. No particular limitation is imposed on the cationic moiety M_{Y} so long as the structural moiety Y can form the acidic moiety represented by HA_{Y} upon irradiation with actinic rays or radiation, and the definition and preferred modes of the cationic moiety M_{Y} are as described above for the cationic moiety in the onium salt (I).

### (Structure of Onium Salt (II))

The onium salt (II) is preferably a compound represented by formula (II-A). An Mₐ⁺Aₐ⁻ moiety in formula (II-A) includes the structural moiety W.

Mₐ⁺Aₐ⁻-Lₐ-Rₐ Formula(II-A)

Mₐ⁺ represents an organic cation. The organic cation is preferably the above-described organic cation represented by formula (ZaI) (the cation (Za1)) or the above-described organic cation represented by formula (ZaII) (the cation (ZaII)).

Aₐ⁻ represents a group represented by any of formulas (B-1) to (B-8).

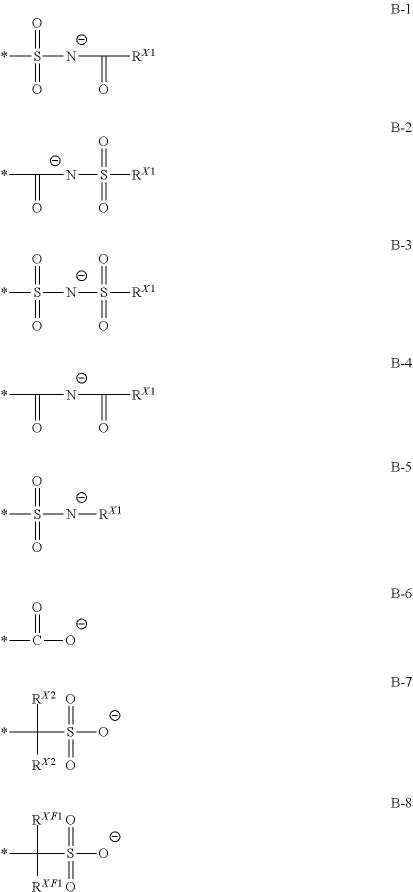

R^{x1} represents an organic group.

R^{x1} is preferably a linear, branched, or cyclic alkyl group or an aryl group.

The number of carbon atoms in the alkyl group is preferably 1 to 15 and more preferably 1 to 10.

The alkyl group may have a substituent. The substituent is preferably a fluorine atom or a cyano group. When the alkyl group has a fluorine atom as a substituent, the alkyl group may be a perfluoroalkyl group.

In the alkyl group, any carbon atom may be replaced with a carbonyl group.

The aryl group is preferably a phenyl group or a naphthyl group and more preferably a phenyl group.

The aryl group may have a substituent. The substituent is preferably a fluorine atom, a perfluoroalkyl group (having, for example, 1 to 10 carbon atoms and preferably 1 to 6 carbon atoms) or a cyano group.

R^{x2} represents a hydrogen atom or a substituent other than a fluorine atom and perfluoroalkyl groups.

The substituent represented by R^{x2} and other than a fluorine atom and perfluoroalkyl groups is preferably an alkyl group (a linear, branched, or cyclic alkyl group) other than perfluoroalkyl groups.

The number of carbon atoms in the alkyl group is preferably 1 to 15 and more preferably 1 to 10.

Preferably, the alkyl group has no fluorine atom. Specifically, when the alkyl group has a substituent, the substituent is preferably different from a fluorine atom.

Each R^{XF1} represents a hydrogen atom, a fluorine atom, or a perfluoroalkyl group. However, at least one of R^{XF1}s represents a fluorine atom or a perfluoroalkyl group.

The number of carbon atoms in the perfluoroalkyl group represented by R^{XF1} is preferably 1 to 15, more preferably 1 to 10, and still more preferably 1 to 6.

Lₐ represents a single bond or a divalent linking group.

No particular limitation is imposed on the divalent linking group represented by Lₐ. Lₐ is, for example, a group selected from the group consisting of —CO—, —NH—, —O—, —S—, —SO—, —SO₂—, and alkylene groups (which have preferably 1 to 10 carbon atoms and may be linear or branched) or is a combination of two or more of them.

The alkylene group may be substituted with a substituent (such as a fluorine atom).

No particular limitation is imposed on the monovalent organic group represented by Rₐ. Examples of the monovalent organic group include fluoroalkyl groups (having preferably 1 to 10 carbon atoms and more preferably 1 to 6 carbon atoms) and organic groups including a ring structure. In particular, the monovalent organic group is preferably a cyclic organic group.

Examples of the cyclic organic group include alicyclic groups, aryl groups, and heterocyclic groups.

The alicyclic group may be a monocyclic alicyclic group or a polycyclic alicyclic group. Examples of the monocyclic alicyclic group include monocyclic cycloalkyl groups such as a cyclopentyl group, a cyclohexyl group, and a cyclooctyl group. Examples of the polycyclic alicyclic group include polycyclic cycloalkyl groups such as a norbornyl group, a tricyclodecanyl group, a tetracyclodecanyl group, a tetracyclododecanyl group, and an adamantyl group. Of these, alicyclic groups having 7 or more carbon atoms and a bulky structure such as a norbornyl group, a tricyclodecanyl group, a tetracyclodecanyl group, a tetracyclododecanyl group, and an adamantyl group are preferred.

In the alicyclic group, any carbon atom may be replaced with a carbonyl group.

The aryl group may be a monocyclic or polycyclic aryl group. Examples of such an aryl group include a phenyl group, a naphthyl group, a phenanthryl group, and an anthryl group.

The heterocyclic group may be a monocyclic or polycyclic heterocyclic group. When the heterocyclic group is a polycyclic heterocyclic group, diffusion of the acid can be further reduced. The heterocyclic group may or may not have aromaticity. Examples of the heterocycle having aromaticity include a furan ring, a thiophene ring, a benzofuran ring, a benzothiophene ring, a dibenzofuran ring, a dibenzothiophene ring, and a pyridine ring. Examples of the heterocycle having no aromaticity include a tetrahydropyran ring, lactone rings, sultone rings, and a decahydroisoquinoline ring. Examples of the lactone rings and the sultone rings include lactone structures and sultone structures exemplified for a resin described later. The heterocycle in the heterocyclic group is preferably a furan ring, a thiophene ring, a pyridine ring, or a decahydroisoquinoline ring.

The above cyclic organic group may have a substituent. Examples of the substituent include alkyl groups (which have preferably 1 to 12 carbon atoms and may be linear or branched), cycloalkyl groups (which may be monocyclic, polycyclic, or spirocyclic and have preferably 3 to 20 carbon atoms), aryl groups (having preferably 6 to 14 carbon atoms), a hydroxy group, alkoxy groups, ester groups, amido groups, urethane groups, ureide groups, thioether groups, sulfonamido groups, and sulfonate groups. Carbon included in the cyclic organic group (carbon contributing to the formation of the ring) may be carbonyl carbon.

The onium salt (II) is also preferably a compound represented by formula (II-B). In formula (II-B), M_{b}⁺ and A_{b}⁻ form a structure including the structural moiety W.

(R_{b})ₘ-M_{b}⁺-L_{b}-A_{b}⁻ Formula (II-B)

M_{b}⁺ represents a sulfur ion (S⁺) or an iodine ion (I⁺).

m represents 1 or 2. When M_{b}⁺ is a sulfur ion, m is 2. When M_{b}⁺ is an iodine ion, m is 1.

R_{b} or R_{b}'s each independently represent an alkyl group optionally including a heteroatom, an alkenyl group optionally including a heteroatom, an aryl group, or a heteroaryl group. When m is 2, the two R_{b}'s may be bonded together to form a ring.

No particular limitation is imposed on the alkyl and alkenyl groups represented by R_{b} and optionally including a heteroatom. Examples of the alkyl and alkenyl groups include alkyl groups having 1 to 20 carbon atoms (preferably 1 to 10 carbon atoms) in which —CH₂— may be replaced with a heteroatom and alkenyl groups having 1 to 20 carbon atoms (preferably 2 to 10 carbon atoms) in which —CH₂— may be replaced with a heteroatom. Examples of the heteroatom include an oxygen atom, a nitrogen atom, and a sulfur atom.

The alkyl or alkenyl group represented by R_{b} and optionally including a heteroatom may be linear, branched, or cyclic.

The alkyl or alkenyl group represented by R_{b} and optionally including a heteroatom may have a substituent. Examples of the substituent include aryl groups (having preferably 6 to 14 carbon atoms), a hydroxy group, alkoxy groups, ester groups, amido groups, urethane groups, ureide groups, thioether groups, sulfonamido groups, and sulfonate groups.

The aryl group represented by R_{b} may be a monocyclic aryl group or may be a polycyclic aryl group. Examples of the aryl group include a phenyl group, a naphthyl group, a phenanthryl group, and an anthryl group.

The heteroaryl group represented by R_{b} may be a monocyclic heteroaryl group or may be a polycyclic heteroaryl group. When the heteroaryl group is a polycyclic heteroaryl group, diffusion of the acid can be further reduced. Examples of the aromatic heterocyclic ring included in the heteroaryl group include a furan ring, a thiophene ring, a benzofuran ring, a benzothiophene ring, a dibenzofuran ring, a dibenzothiophene ring, and a pyridine ring.

The aryl and heteroaryl groups represented by R_{b} may have a substituent. Examples of the substituent include alkyl groups (which may be linear or branched and have preferably 1 to 12 carbon atoms), cycloalkyl groups (which may be monocyclic, polycyclic, or spirocyclic and have preferably 3 to 20 carbon atoms), aryl groups (having preferably 6 to 14 carbon atoms), a hydroxy group, alkoxy groups, ester groups, amido groups, urethane groups, ureide groups, thioether groups, sulfonamido groups, and sulfonate groups.

No particular limitation is imposed on the divalent linking group represented by Lb. The divalent linking group is, for example, at least one selected from the group consisting of —CO—, —NH—, —O—, —S—, —SO—, —SO₂—, alkylene groups (which have preferably 1 to 10 carbon atoms and may be linear or branched) and arylene groups (having preferably 6 to 10 carbon atoms) or is a combination of two or more of them.

Any hydrogen atom in the alkylene and arylene groups may be replaced with a substituent (such as a fluorine atom).

A_{b}⁻ represents a group represented by any of formulas (B-1) to (B-8) above.

The onium salt (II) is also preferably a compound represented by formula (Ia-1). In formula (Ia-1), at least one of an M₁₁+A₁₁⁻ moiety or an A₁₂⁻M₁₂⁺ moiety includes the structural moiety W.

M₁₁⁺A₁₁⁻L₁-A₁₂-M₁₂⁺ (Ia-1)

In formula (Ia-1), M₁₁⁺ and M₁₂⁺ each represent an organic cation. The organic cation is preferably the above-described organic cation represented by formula (ZaI) (the cation (Za1)) or the above-described organic cation represented by formula (ZaII) (the cation (ZaII)).

In formula (Ia-1), A₁₁⁻ and A₁₂⁻ each independently represent a group represented by any of formulas (B-1) to (B-8).

In formula (Ia-1), L₁ represents a divalent linking group. No particular limitation is imposed on the divalent linking group represented by L₁. The divalent linking group is, for example, at least one selected from the group consisting of —CO—, —NH—, —O—, —S—, —SO—, —SO₂—, alkylene groups (which have preferably 1 to 10 carbon atoms and may be linear or branched), cycloalkylene groups (which may be monocyclic, polycyclic, or spirocyclic and have preferably 5 to 20 carbon atoms), and arylene groups (having preferably 6 to 10 carbon atoms) or is a combination of two or more of them.

Any hydrogen atom in the alkylene, cycloalkylene, and arylene groups may be replaced with a substituent (such as a fluorine atom).

The onium salt (II) is also preferably a compound represented by any of formulas (Ia-2) to (Ia-4).

In formula (Ia-2), at least one of an M₂₂⁺A₂₂⁻ moiety, an A₂₁ₐ⁻M₂₁ₐ⁺ moiety, or an A₂₁⁻ M₂₁ₑ⁺ moiety includes the structural moiety W.

In formula (Ia-4), at least one of an M₄₂⁺A₄₂⁻ moiety, an A₄₁ₐ⁻M₄₁ₐ⁺ moiety, or an A₄₁⁻ M₄₁ₑ⁺ moiety includes the structural moiety W.

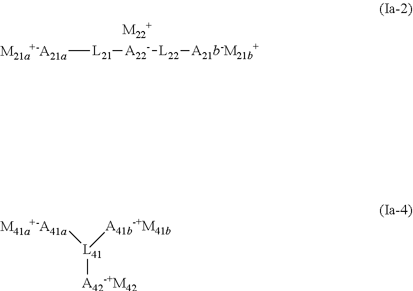

In formula (Ia-2), M₂₂⁺, M₂₁ₐ⁺ and M₂₁ₑ⁺ each represent an organic cation. The organic cation is preferably the above-described organic cation represented by formula (ZaI) (the cation (Za1)) or the above-described organic cation represented by formula (ZaII) (the cation (ZaII)).

In formula (Ia-2), A₂₁ₐ⁻ and A₂₁⁻ each independently represent a group represented by any of formulas (B-1) to (B-8). A₂₂⁻ represents a group represented by any of formulas (II)-1 to (II)-4.

In formula (Ia-2), L₂₁ and L₂₂ each represent a divalent linking group. No particular limitation is imposed on the divalent linking groups represented by L₂₁ and L₂₂. Examples of the divalent linking groups include the groups described above for L_{b}. L₂₁ and L₂₂ may be the same or different.

In formula (Ia-4), M₄₂⁺, M₄₁ₐ⁺, and M₄₁ₑ⁺ each represent an organic cation. The organic cation is preferably the above-described organic cation represented by formula (ZaI) (the cation (Za1)) or the above-described organic cation represented by formula (ZaII) (the cation (ZaII)).

In formula (Ia-4), A₄₁ₐ⁻, A_{41b}⁻, and A₄₂⁻ each independently represent a group represented by any of formulas (B-1) to (B-8).

In formula (Ia-4), L₄₁ represents a trivalent linking group. Examples of the trivalent organic group include a trivalent organic group represented by formula (L3).

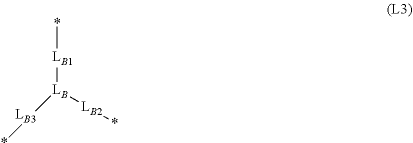

In formula (L3), L_{B} represents a trivalent hydrocarbon ring group or a trivalent heterocyclic group. * represents a bonding position.

The above hydrocarbon ring group may be an aromatic hydrocarbon ring group or may be an aliphatic hydrocarbon ring group. The number of carbon atoms included in the hydrocarbon ring group is preferably 6 to 18 and more preferably 6 to 14. The above heterocyclic group may be an aromatic heterocyclic group or may be an aliphatic heterocyclic group. The heterocycle is preferably a 5- to 10-membered ring, more preferably a 5- to 7-membered ring, and still more preferably a 5- to 6-membered ring, each of which has at least one nitrogen, oxygen, sulfur, or Se atom in its ring structure.

L_{B} is preferably a trivalent hydrocarbon ring group and more preferably a benzene ring group or an adamantane ring group. The benzene ring group and the adamantane ring group may each have a substituent. The substituent is, for example, a halogen atom (preferably a fluorine atom).

In formula (L₃), L_{B1} to L_{B3} each independently represent a single bond or a divalent linking group. Examples of the divalent linking groups represented by L_{B1} to L_{B3} include —CO—, —NR—, —O—, —S—, —SO—, —SO₂—, alkylene groups (which have preferably 1 to 6 carbon atoms and may be linear or branched), cycloalkylene groups (having preferably 3 to 15 carbon atoms), alkenylene groups (having preferably 2 to 6 carbon atoms), divalent aliphatic heterocyclic groups (which are preferably 5- to 10-membered rings, more preferably 5- to 7-membered rings, and still more preferably 5- to 6-membered rings, each of which has at least one nitrogen, oxygen, sulfur, or Se atom in its ring structure), divalent aromatic heterocyclic groups (which are preferably 5- to 10-membered rings, more preferably 5- to 7-membered rings, and still more preferably 5- to 6-membered rings, each of which has at least one nitrogen, oxygen, sulfur, or Se atom in its ring structure), divalent aromatic hydrocarbon ring groups (which are preferably 6- to 10-membered rings and more preferably 6-membered rings), and divalent linking groups obtained by combining any of them. Examples of R include a hydrogen atom and monovalent organic groups. The monovalent organic group is, for example, an alkyl group (having preferably 1 to 6 carbon atoms).

The above alkylene, cycloalkylene, alkenylene, divalent aliphatic heterocyclic, divalent aromatic heterocyclic, and divalent aromatic hydrocarbon ring groups may each have a substituent. The substituent is, for example, a halogen atoms (preferably a fluorine atom).

The divalent linking groups represented by L_{B1} to L_{B3} are each preferably —CO—, —NR—, —O—, —S—, —SO—, —SO₂—, an alkylene group optionally having a substituent, or a divalent linking group obtained by combining any of them.

The molecular weight of the onium salt (II) is preferably 100 to 10000, more preferably 100 to 2500, and still more preferably 100 to 1500.

The content of the onium salt (II) with respect to the total mass of the solids in the resist composition is preferably 2% by mass or more, preferably 5% by mass or more, still more preferably 10% by mass or more, and yet more preferably 15% by mass or more. The upper limit of the content of the onium salt (II) with respect to the total mass of the solids in the resist composition is preferably 80% by mass or less, more preferably 70% by mass or less, and still more preferably 60% by mass or less.

One onium salt (II) may be used alone, or two or more onium salts (II) may be used. When two or more onium salts (II) are used, it is preferable that the total content of the onium salts (II) falls within the above preferred range.

The total content of the onium salt (I) and the onium salt (II) with respect to the total mass of the solids in the resist composition is preferably 10% by mass or more, more preferably 15% by mass or more, still more preferably 20% by mass or more, and yet more preferably 30% by mass or more. The upper limit of the total content with respect to the total mass of the solids in the resist composition is preferably 80% by mass or less, more preferably 70% by mass or less, and still more preferably 60% by mass or less.

### [Additional Photoacid Generator]

The resist composition may contain, in addition to the onium salt (I) and the onium salt (II), a compound (an additional photoacid generator) that generates an acid upon irradiation with actinic rays or radiation.

### [Acid Decomposable Resin]

The resist composition includes the acid decomposable resin.

The acid decomposable resin is hereinafter referred to simply as a “resin (A).”

Preferably, the acid decomposable resin has an acid-decomposable group. The “acid-decomposable group” means a group that is decomposed by the action of an acid to generate a polar group.

Typically, in the pattern forming method of the invention, when a developer used is an alkali developer, a positive-type pattern is preferably formed. When the developer used is an organic-based developer, a negative-type pattern is preferably formed.

Repeating units that can be included in the acid decomposable resin will be described.

### (Repeating Unit Having Acid-Decomposable Group)

Preferably, the acid-decomposable group has a structure in which the polar group is protected by a leaving group that leaves by the action of an acid. Specifically, it is preferable that the acid decomposable resin has a repeating unit having a group that is decomposed by the action of an acid to generate a polar group. The resin having this repeating unit is increased in polarity by the action of an acid. The degree of solubility in an alkali developer thereby increases, and the degree of solubility in an organic solvent decreases.

The polar group that is generated when the acid-decomposable group is decomposed by the action of an acid is preferably an alkali-soluble group.

Examples of the alkali-soluble group include: acidic groups such as a carboxy group, phenolic hydroxy groups, fluorinated alcohol groups, sulfonic acid groups, phosphoric acid groups, sulfonamido groups, sulfonylimido groups, (alkylsulfonyl)(alkylcarbonyl)methylene groups, (alkylsulfonyl)(alkylcarbonyl)imido groups, bis(alkylcarbonyl)methylene groups, bis(alkylcarbonyl)imido groups, bis(alkylsulfonyl)methylene groups, bis(alkylsulfonyl)imido groups, tris(alkylcarbonyl)methylene groups, and tris(alkylsulfonyl)methylene groups; and alcoholic hydroxyl groups.

As described above, it is preferable that the acid-decomposable group has a structure in which the polar group is protected by the leaving group that leaves by the action of an acid.

Examples of the leaving group that leaves by the action of an acid include groups represented by formulas (Y1) to (Y4).

—C(Rx₁)(Rx₂)(Rx₃) Formula (Y1):

—C(═O)OC(Rx₁)(Rx₂)(Rx₃) Formula (Y2):

—C(R₃₆)(R₃₇)(OR₃₈) Formula (Y3):

—C(Rn)(H)(Ar) Formula (Y4):

In general formulas (Y1) and (Y2), Rx₁ to Rx₃ each independently represent an alkyl group (linear or branched alkyl group), a cycloalkyl group (monocyclic or polycyclic cycloalkyl group), an alkenyl group (linear or branched alkenyl group), an aryl group (monocyclic or polycyclic aryl group), or a heteroaryl group (monocyclic or polycyclic heteroaryl group). When all of Rx₁ to Rx₃ are alkyl groups (linear or branched alkyl groups), it is preferable that at least two selected from the group consisting of Rx₁ to Rx₃ are each a methyl group.

In particular, it is preferable that Rx₁ to Rx₃ each independently represent a linear or branched alkyl group, and it is more preferable that Rx₁ to Rx₃ each independently represent a linear alkyl group.

Two selected from the group consisting of Rx₁ to Rx₃ may be bonded together to form a monocyclic or polycyclic ring.

The alkyl group represented by each of Rx₁ to Rx₃ is preferably an alkyl group having 1 to 5 carbon atoms such as a methyl group, an ethyl group, a n-propyl group, an isopropyl group, a n-butyl group, an isobutyl group, or a t-butyl group.

The cycloalkyl group represented by each of Rx₁ to Rx₃ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group.

The aryl group represented by each of Rx₁ to Rx₃ is, for example, preferably an aryl group having 6 to 10 carbon atoms, and examples thereof include a phenyl group, a naphthyl group, and an anthryl group.

The heteroaryl group represented by each of Rx₁ to Rx₃ is, for example, preferably a heteroaryl group having 4 to 10 carbon atoms.

The alkenyl group represented by each of Rx₁ to Rx₃ is preferably a vinyl group. The ring formed by bonding two selected from the group consisting of Rx₁ to Rx₃ is preferably a cycloalkyl group. The cycloalkyl group formed by bonding two selected from the group consisting of Rx₁ to Rx₃ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group and is more preferably a monocyclic cycloalkyl group having 5 to 6 carbon atoms.

For example, one methylene group included in the ring in the cycloalkyl group formed by bonding two selected from the group consisting of Rx₁ to Rx₃ may be replaced with a heteroatom such as an oxygen atom, a group including a heteroatom such as a carbonyl group, or a vinylidene group. In any of these cycloalkyl groups, at least one ethylene group included in the cycloalkane ring may be replaced with a vinylene group.

In the group represented by general formula (Y1) or general formula (Y2), it is preferable that, for example, Rx₁ is a methyl group or an ethyl group and that Rx₂ and Rx₃ are bonded together to form the cycloalkyl group described above.

When the resist composition is, for example, a resist composition for EUV exposure, it is also preferable that the alkyl, cycloalkyl, alkenyl, aryl, and heteroaryl groups represented by Rx₁ to Rx₃ and the ring formed by bonding two selected from the group consisting of Rx₁ to Rx₃ each further have a fluorine atom or an iodine atom as a substituent.

In formula (Y3), R₃₆ to R₃₈ each independently represent a hydrogen atom or a monovalent organic group. R₃₇ and R₃₈ may be bonded together to form a ring. Examples of the monovalent organic group include alkyl groups, cycloalkyl groups, aryl groups, aralkyl groups, and alkenyl groups. It is also preferable that R₃₆ is a hydrogen atom.

The alkyl, cycloalkyl, aryl, and aralkyl groups described above may each include a heteroatom such as an oxygen atom and/or a group including a heteroatom such as a carbonyl group. For example, in the alkyl, cycloalkyl, aryl, and aralkyl groups described above, at least one methylene group may be replaced with a heteroatom such as an oxygen atom and/or a group including a heteroatom such as a carbonyl group.

R₃₈ may be bonded to another substituent included in the main chain of the repeating unit to form a ring. The group formed by bonding R₃₈ and another substituent included in the main chain of the repeating unit is preferably an alkylene group such as a methylene group.

When the resist composition is, for example, a resist composition for EUV exposure, it is also preferable that the monovalent organic groups represented by R₃₆ to R₃₈ and the group formed by bonding R₃₇ and R₃₈ together each further have a fluorine atom or an iodine atom as a substituent.

Formula (Y3) is preferably a group represented by the following formula (Y3-1).

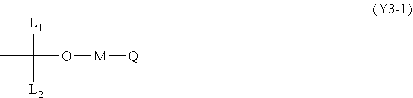

L₁ and L₂ each independently represent a hydrogen atom, an alkyl group, a cycloalkyl group, an aryl group, or a group formed by combining any of them (for example, a group formed by combining an alkyl group and an aryl group).

M represents a single bond or a divalent linking group.

Q represents an alkyl group optionally including a heteroatom, a cycloalkyl group optionally including a heteroatom, an aryl group optionally including a heteroatom, an amino group, an ammonium group, a mercapto group, a cyano group, an aldehyde group, or a group formed by combining any of them (for example, a group formed by combining an alkyl group and a cycloalkyl group).

In the alkyl and cycloalkyl groups, for example, one methylene group may be replaced with a heteroatom such as an oxygen atom or a group including a heteroatom such as a carbonyl group.

It is preferable that one of L₁ or L₂ is a hydrogen atom and that the other is an alkyl group, a cycloalkyl group, an aryl group, or a group formed by combining an alkylene group and an aryl group.

At least two selected from the group consisting of Q, M, and L₁ may be bonded together to form a ring (preferably a 5-membered or 6-membered ring).

From the viewpoint of obtaining a finer pattern, L₂ is preferably a secondary or tertiary alkyl group and more preferably a tertiary alkyl group. Examples of the secondary alkyl group include an isopropyl group, a cyclohexyl group, and a norbornyl group, and examples of the tertiary alkyl group include a tert-butyl group and an adamantane group. In these modes, since Tg (glass transition temperature) and activation energy are high, high film hardness is obtained, and the occurrence of fogging can be reduced.

When the resist composition is, for example, a resist composition for EUV exposure, it is also preferable that the alkyl, cycloalkyl, and aryl groups represented by L₁ and L₂ and a group formed by combining any of these groups each further have a fluorine atom or an iodine atom as a substituent. It is also preferable that the alkyl, cycloalkyl, aryl, and aralkyl groups each include a heteroatom such as an oxygen atom other than a fluorine atom and an iodine atom (i.e., in the alkyl, cycloalkyl, aryl, and aralkyl groups, for example, one methylene group is replaced with a heteroatom such as an oxygen atom or a group including a heteroatom such as a carbonyl group).

When the resist composition is, for example, a resist composition for EUV exposure, it is also preferable that, in the alkyl group optionally including a heteroatom, the cycloalkyl group optionally including a heteroatom, the aryl group optionally including a heteroatom, the amino group, the ammonium group, the mercapto group, the cyano group, and the aldehyde group that are represented by Q and a combination of any of these groups, the heteroatom is one selected from the group consisting of a fluorine atom, an iodine atom, and an oxygen atom.

In formula (Y4), Ar represents an aromatic ring group. Rn represents an alkyl group, a cycloalkyl group, or an aryl group. Rn and Ar may be bonded together to form a non-aromatic ring. Ar is preferably an aryl group.

When the resist composition is, for example, a resist composition for EUV exposure, it is also preferable that the aromatic ring group represented by Ar and the alkyl, cycloalkyl, or aryl group represented by Rn each have a fluorine atom or an iodine atom as a substituent.

When, in the leaving group protecting the polar group, a non-aromatic ring is bonded directly to the polar group (or its residue), it is also preferable that a ring member atom adjacent to the ring member atom bonded directly to the polar group (or its residue) in the non-aromatic ring does not have a halogen atom such as a fluorine atom as a substituent, because the repeating unit can have good acid-decomposability.

The leaving group that leaves by the action of an acid may also be a 2-cyclopentenyl group having a substituent (e.g., an alkyl group) such as a 3-methyl-2-cyclopentenyl group or a cyclohexyl group having a substituent (e.g., an alkyl group) such as a 1,1,4,4-tetramethylcyclohexyl group.

The repeating unit having an acid-decomposable group is also preferably a repeating unit represented by formula (A).

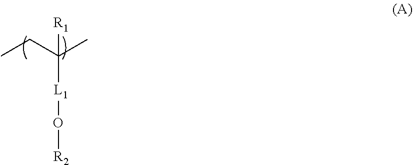

L₁ represents a divalent linking group optionally having a fluorine atom or an iodine atom, and R₁ represents a hydrogen atom, a fluorine atom, an iodine atom, an alkyl group optionally having a fluorine atom or an iodine atom, or an aryl group optionally having a fluorine atom or an iodine atom. R₂ represents a leaving group that optionally has a fluorine atom or an iodine atom and leaves by the action of an acid. At least one of L₁, R₁, or R₂ has a fluorine atom or an iodine atom.

L₁ represents a divalent linking group optionally having a fluorine atom or an iodine atom. Examples of the divalent linking group optionally having a fluorine atom or an iodine atom include —CO—, —O—, —S—, —SO—, —SO₂—, hydrocarbon groups optionally having a fluorine atom or an iodine atom (such as alkylene groups, cycloalkylene groups, alkenylene groups, and arylene groups), and linking groups formed by linking a plurality of groups selected from the above groups. In particular, L₁ is preferably —CO—, an arylene group, or -arylene group-fluorine or iodine atom-containing alkylene group- and more preferably —CO— or -arylene group-fluorine or iodine atom-containing alkylene group-.

The arylene group is preferably a phenylene group.

The alkylene group may by a linear or branched alkylene group. No particular limitation is imposed on the number of carbon atoms in the alkylene group, but the number of carbon atoms is preferably 1 to 10 and more preferably 1 to 3.

No particular limitation is imposed on the total number of fluorine or iodine atoms included in the fluorine or iodine atom-containing alkylene group, but the total number of fluorine or iodine atoms is preferably two or more, more preferably 2 to 10, and still more preferably 3 to 6.

R₁ represents a hydrogen atom, a fluorine atom, an iodine atom, an alkyl group optionally having a fluorine atom or an iodine atom, or an aryl group optionally having a fluorine atom or an iodine atom.

The alkyl group may be a linear or branched alkyl group. No particular limitation is imposed on the number of carbon atoms in the alkyl group, but the number of carbon atoms is preferably 1 to 10 and more preferably 1 to 3.

No particular limitation is imposed on the total number of fluorine or iodine atoms included in the fluorine or iodine atom-containing alkyl group, but the total number of fluorine or iodine atoms is preferably 1 or more, more preferably 1 to 5, and still more preferably 1 to 3.

The alkyl group may include a heteroatom such as an oxygen atom other than halogen atoms.

R₂ represents a leaving group that leaves by the action of an acid and that optionally has a fluorine atom or an iodine atom. Examples of the leaving group optionally having a fluorine atom or an iodine atom include leaving groups represented by formulas (Y1) to (Y4) described above and having a fluorine atom or an iodine atom.

It is also preferable that the repeating unit having an acid-decomposable group is a repeating unit represented by formula (AI).

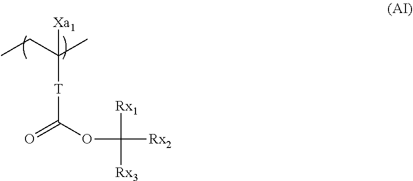

In formula (AI), Xa₁ represents a hydrogen atom or an alkyl group optionally having a substituent. T represents a single bond or a divalent linking group. Rx₁ to Rx₃ each independently represent an alkyl group (linear or branched alkyl group), a cycloalkyl group (monocyclic or polycyclic cycloalkyl group), an alkenyl group (linear or branched alkenyl group), or an aryl group (monocyclic or polycyclic aryl group). However, when all of Rx₁ to Rx₃ are alkyl groups (linear or branched alkyl groups), it is preferable that at least two selected from the group consisting of Rx₁ to Rx₃ are each a methyl group.

Two selected from the group consisting of Rx₁ to Rx₃ may be bonded together to form a monocyclic or polycyclic group (such as a monocyclic or polycyclic cycloalkyl group).

Examples of the alkyl group optionally having a substituent and represented by Xa₁ include a methyl group and a group represented by —CH₂—R₁₁. R₁₁ represents a halogen atom (such as a fluorine atom), a hydroxy group, or a monovalent organic group, and examples thereof include alkyl groups having 5 or less carbon atoms and optionally substituted with a halogen atom, acyl groups having 5 or less carbon atoms and optionally substituted with a halogen atom, and alkoxy groups having 5 or less carbon atoms and optionally substituted with a halogen atom. Rn is preferably an alkyl group having 3 or less carbon atoms and more preferably a methyl group. Xa₁ is preferably a hydrogen atom, a methyl group, a trifluoromethyl group, or a hydroxymethyl group.

Examples of the divalent linking group represented by T include alkylene groups, aromatic ring groups, a —COO-Rt-group, and an —O-Rt-group. In these formulas, Rt represents an alkylene group or a cycloalkylene group.

T is preferably a single bond or a —COO-Rt-group. When T represents a —COO-Rt-group, Rt is preferably an alkylene group having 1 to 5 carbon atoms and more preferably a —CH₂— group, a —(CH₂)₂— group, or a —(CH₂)₃— group.

The alkyl group represented by each of Rx₁ to Rx₃ is preferably an alkyl group having 1 to 4 carbon atoms such as a methyl group, an ethyl group, a n-propyl group, an isopropyl group, a n-butyl group, an isobutyl group, or a t-butyl group.

The cycloalkyl group represented by each of Rx₁ to Rx₃ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group.

The aryl group represented by each of Rx₁ to Rx₃ is preferably an aryl group having 6 to 10 carbon atoms, and examples thereof include a phenyl group, a naphthyl group, and an anthryl group.

The alkenyl group represented by each of Rx₁ to Rx₃ is preferably a vinyl group. The cycloalkyl group formed by bonding two selected from the group consisting of Rx₁ to Rx₃ is preferably a monocyclic cycloalkyl group such as a cyclopentyl group or a cyclohexyl group. The cycloalkyl group is also preferably a polycyclic cycloalkyl group such as a norbornyl group, a tetracyclodecanyl group, a tetracyclododecanyl group, or an adamantyl group. In particular, a monocyclic cycloalkyl group having 5 to 6 carbon atoms is preferred. In the cycloalkyl group formed by bonding two selected from the group consisting of Rx₁ to Rx₃, for example, one methylene group included in the ring may be replaced with a heteroatom such as an oxygen atom, a group including a heteroatom such as a carbonyl group, or a vinylidene group. In each cycloalkyl group, at least one ethylene group included in the cycloalkane ring may be replaced with a vinylene group.

In the repeating unit represented by formula (AI), it is preferable that, for example, Rx₁ is a methyl group or an ethyl group and that Rx₂ and Rx₃ are bonded together to form the cycloalkyl group described above.

When any of the above-described groups has a substituent, examples of the substituent include alkyl groups (having 1 to 4 carbon atoms), halogen atoms, a hydroxy group, alkoxy groups (having 1 to 4 carbon atoms), a carboxy group, and alkoxycarbonyl groups (having 2 to 6 carbon atoms). The number of carbon atoms in the substituent is preferably 8 or less.

The repeating unit represented by formula (AI) is preferably an acid-decomposable tertiary alkyl (meth)acrylate-based repeating unit (a repeating unit in which Xa₁ represents a hydrogen atom or a methyl group and T represents a single bond).

Specific examples of the repeating unit having an acid-decomposable group are shown below, but the present invention is not limited thereto. In the formulas below, Xa₁ represents H, CH₃, CF₃, or CH₂OH, and Rxa and Rxb each independently represent a linear or branched alkyl group having 1 to 5 carbon atoms.

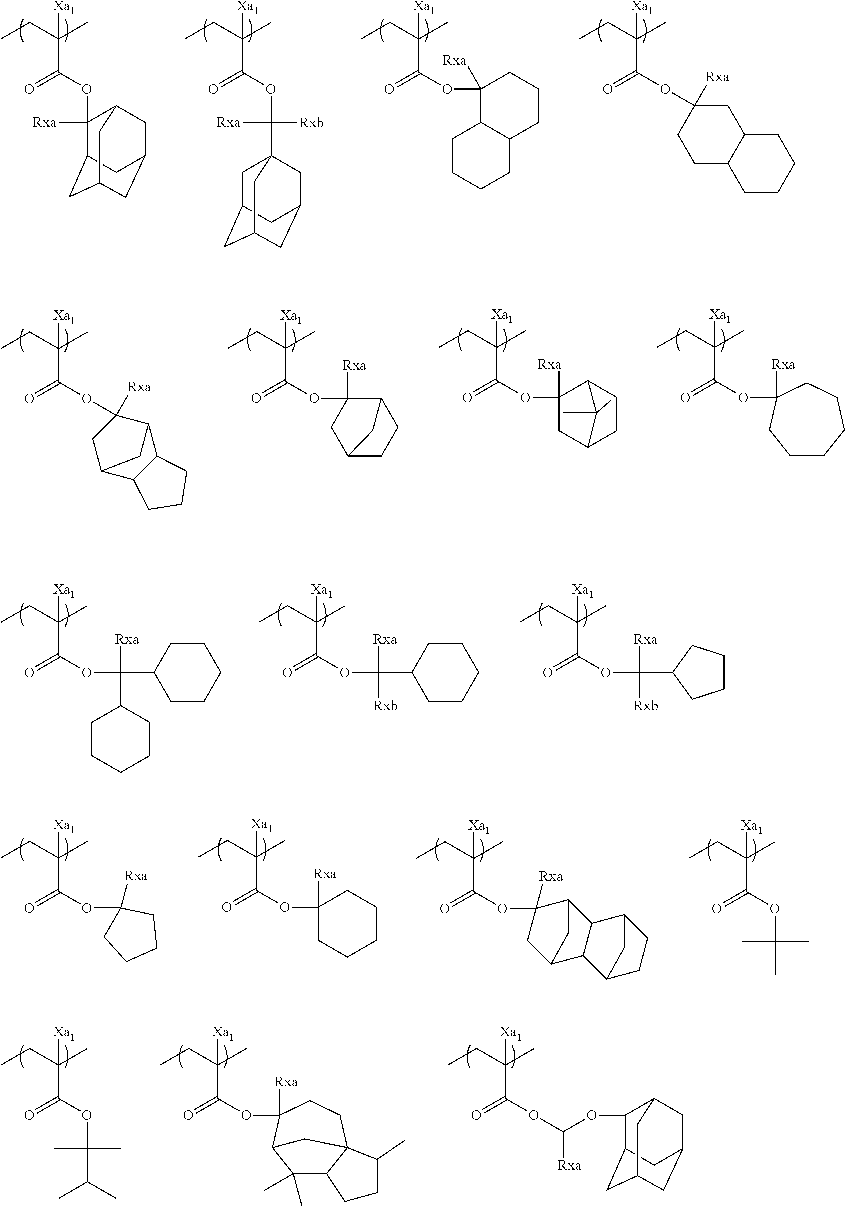

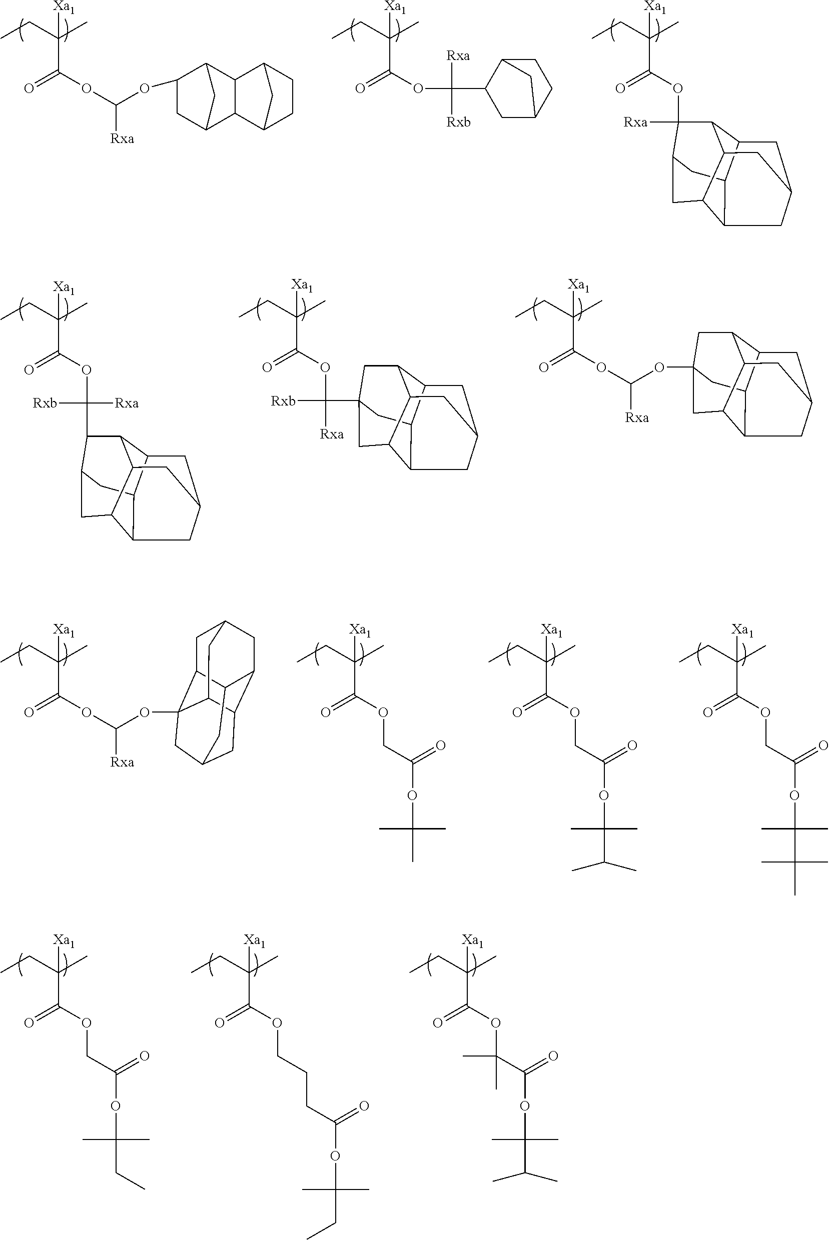

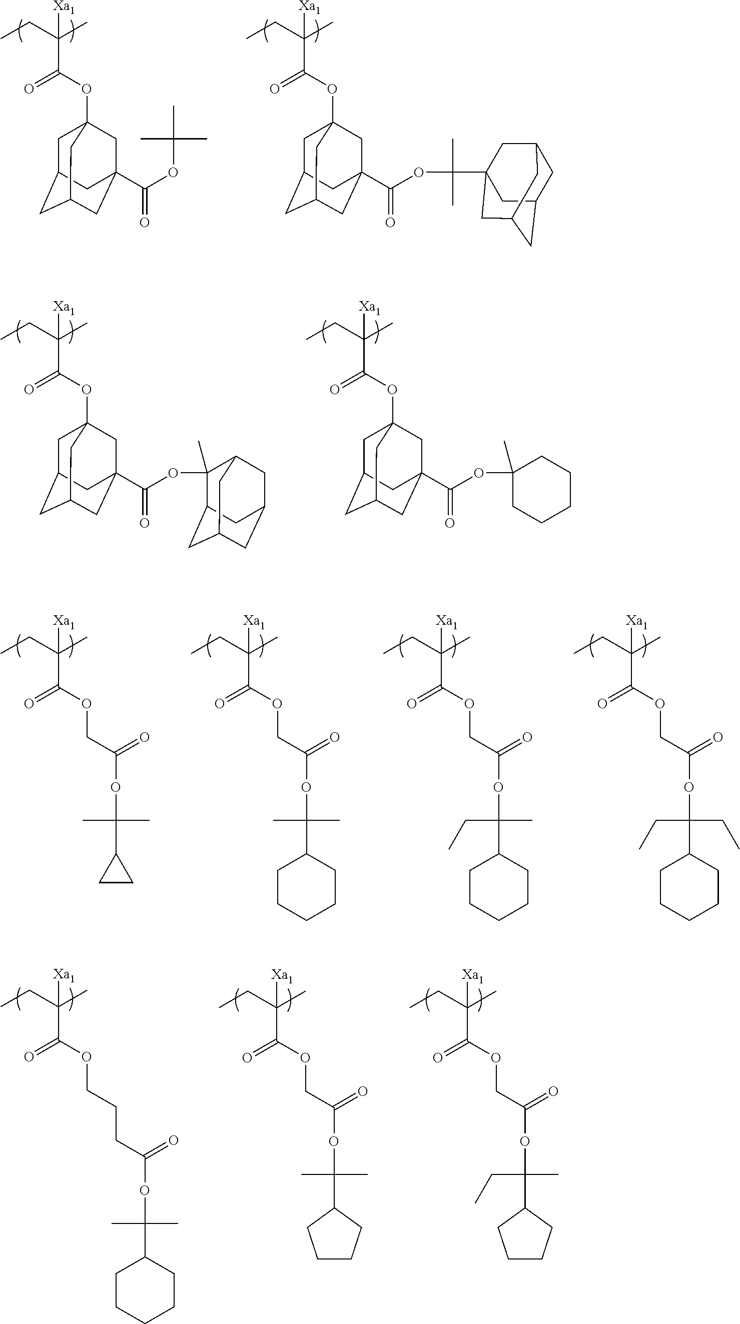

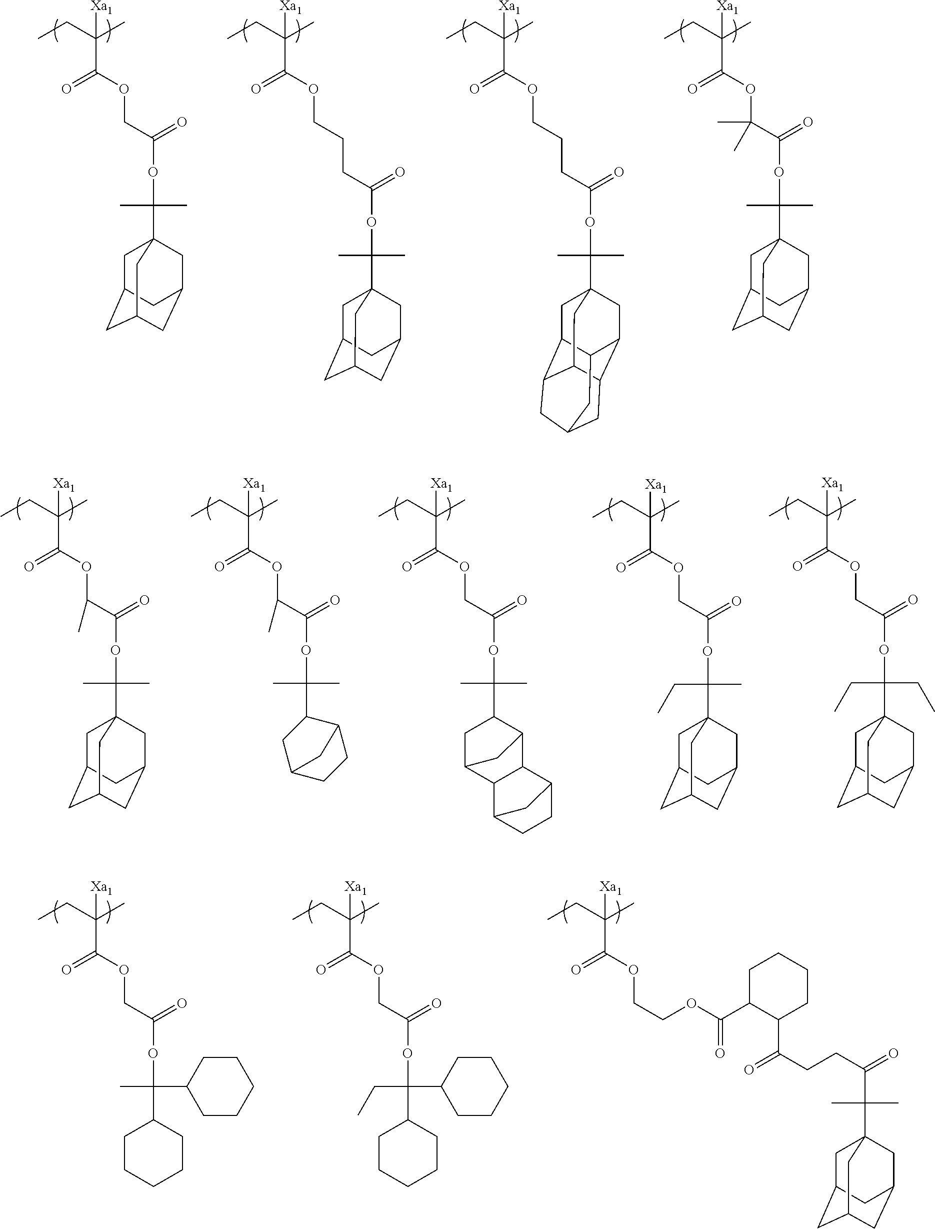

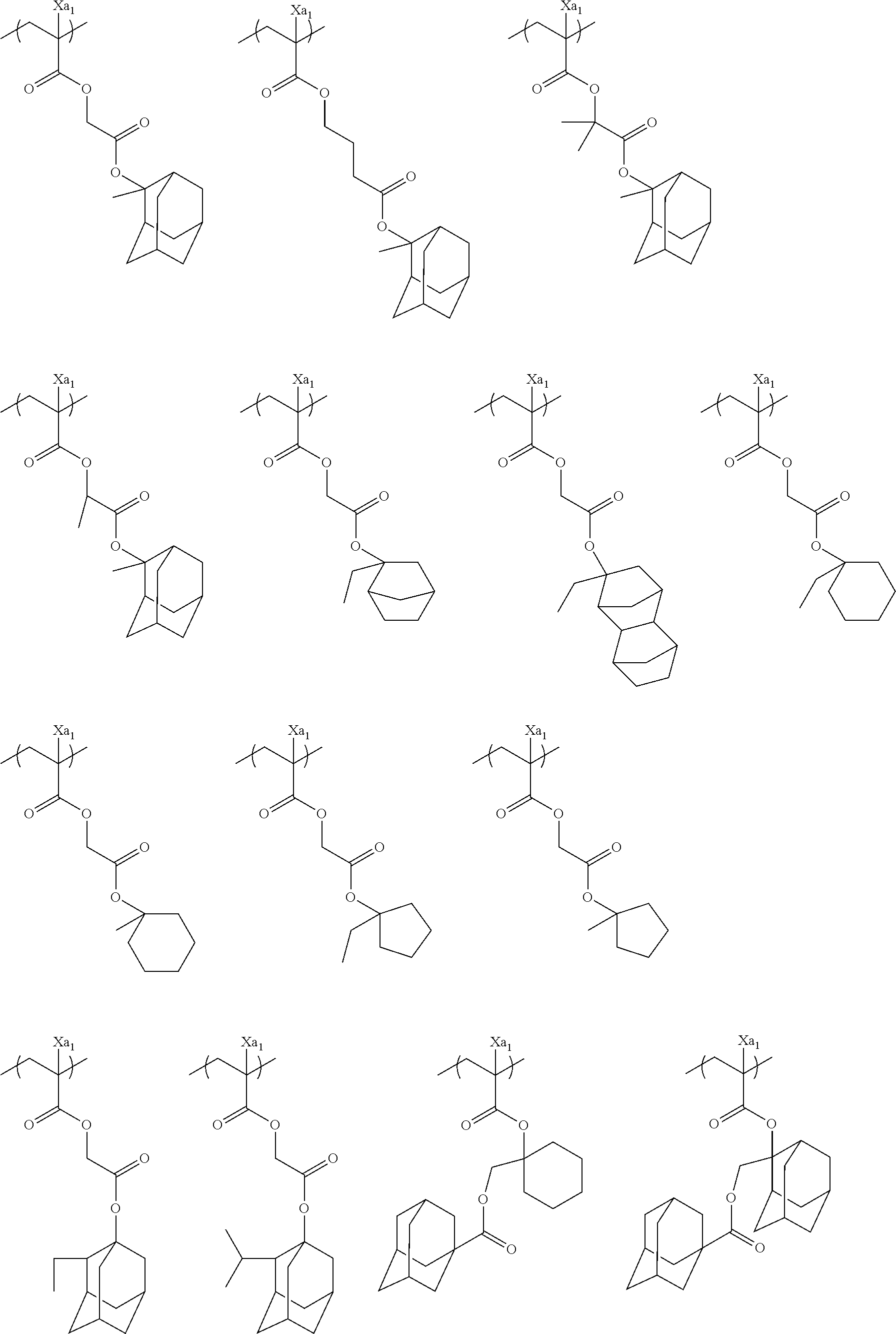

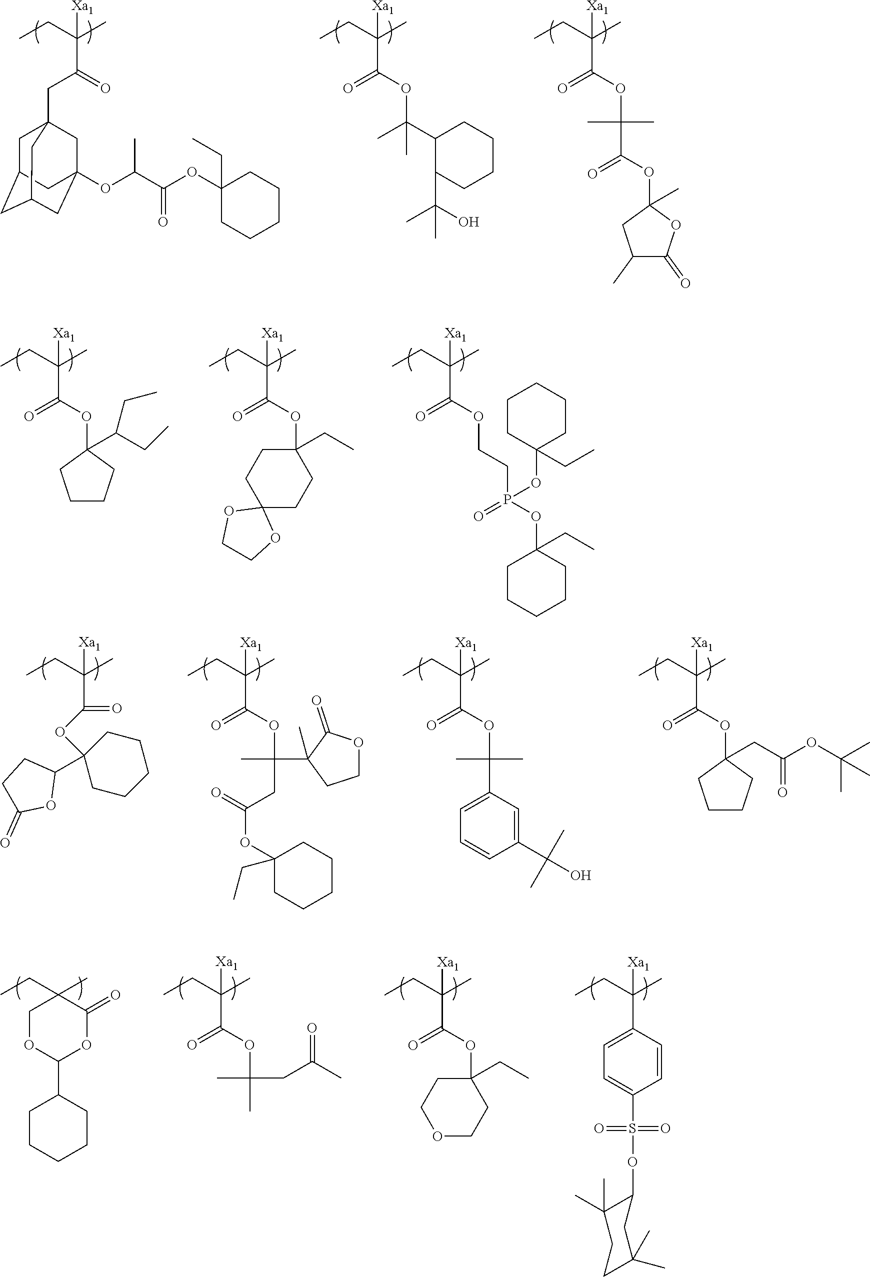

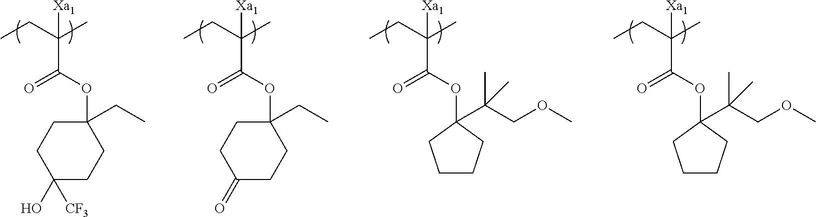

The resin (A) may include as the repeating unit having the acid-decomposable group, a repeating unit having an acid-decomposable group including an unsaturated bond.

The repeating unit having an acid-decomposable group including an unsaturated bond is preferably a repeating unit represented by formula (B).

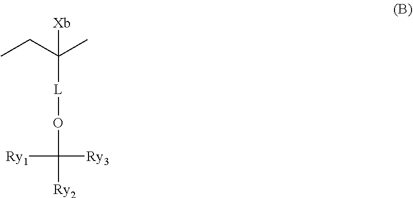

In formula (B), Xb represents a hydrogen atom, a halogen atom, or an alkyl group optionally having a substituent. L represents a single bond or a divalent linking group optionally having a substituent. Ry₁ to Ry₃ each independently represent a linear or branched alkyl group, a monocyclic or polycyclic cycloalkyl group, an alkenyl group, an alkynyl group, or a monocyclic or polycyclic aryl group. However, at least one of Ry₁, Ry₂, or Ry₃ represents an alkenyl group, an alkynyl group, a monocyclic or polycyclic cycloalkenyl group, or a monocyclic or polycyclic aryl group.

Two selected from the group consisting of Ry₁ to Ry₃ may be bonded together to form a monocyclic or polycyclic ring (such as a monocyclic or polycyclic cycloalkyl group or a monocyclic or polycyclic cycloalkenyl group).

The alkyl group optionally having a substituent and represented by Xb is, for example, a methyl group or a group represented by —CH₂—R₁₁. Rn represents a halogen atom (such as a fluorine atom), a hydroxy group, or a monovalent organic group, and examples thereof include alkyl groups having 5 or less carbon atoms and optionally substituted with a halogen atom, acyl groups having 5 or less carbon atoms and optionally substituted with a halogen atom, and alkoxy groups having 5 or less carbon atoms and optionally substituted with a halogen atom. R₁ is preferably an alkyl group having 3 or less carbon atoms and more preferably a methyl group. Xb is preferably a hydrogen atom, a fluorine atom, a methyl group, a trifluoromethyl group, or a hydroxymethyl group.

Examples of the divalent linking group represented by L include an -Rt- group, a —CO— group, a —COO-Rt- group, a —COO-Rt-CO— group, an -Rt-CO— group, and an —O-Rt- group. In these formulas, Rt represents an alkylene group, a cycloalkylene group, or an aromatic ring group and is preferably an aromatic ring group.

L is preferably an -Rt- group, a —CO— group, a —COO-Rt-CO— group, or an -Rt-CO-group. Rt may have a substituent such as a halogen atom, a hydroxy group, or an alkoxy group. Rt is preferably an aromatic group.

The alkyl group represented by each of Ry₁ to Ry₃ is preferably an alkyl group having 1 to 4 carbon atoms such as a methyl group, an ethyl group, a n-propyl group, an isopropyl group, a n-butyl group, an isobutyl group, or a t-butyl group.

The cycloalkyl group represented by each of Ry₁ to Ry₃ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group.

The aryl group represented by each of Ry₁ to Ry₃ is preferably an aryl group having 6 to 10 carbon atoms, and examples thereof include a phenyl group, a naphthyl group, and an anthryl group.

The alkenyl group represented by each of Ry₁ to Ry₃ is preferably a vinyl group.

The alkynyl group represented by each of Ry₁ to Ry₃ is preferably an ethynyl group.

The cycloalkenyl group represented by each of Ry₁ to Ry₃ is preferably a structure including a monocyclic cycloalkyl group such as a cyclopentyl group or a cyclohexyl group with a double bond present in part of the monocyclic cycloalkyl group.

The cycloalkyl group formed by bonding two selected from the group consisting of Ry₁ to Ry₃ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group. In particular, the cycloalkyl group is more preferably a monocyclic cycloalkyl group having 5 to 6 carbon atoms.

In the cycloalkyl or cycloalkenyl group formed by bonding two selected from the group consisting of Ry₁ to Ry₃, for example, one methylene group included in the ring may be replaced with a heteroatom such as an oxygen atom, a group including a heteroatom such as a carbonyl group, an —SO₂— group, or an —SO₃— group, a vinylidene group, or a combination thereof. In the cycloalkyl or cycloalkenyl group, at least one ethylene group included in the cycloalkane or cycloalkenyl ring may be replaced with a vinylene group.

In the repeating unit represented by formula (B), it is preferable that, for example, Ry₁ is a methyl group, an ethyl group, a vinyl group, an allyl group, or an aryl group and that Ry₂ and Ry₃ are bonded together to form the cycloalkyl or cycloalkenyl group described above.

When any of the groups described above has a substituent, examples of the substituent include alkyl groups (having 1 to 4 carbon atoms), halogen atoms, a hydroxy group, alkoxy groups (having 1 to 4 carbon atoms), a carboxy group, and alkoxycarbonyl groups (having 2 to 6 carbon atoms). The number of carbon atoms in the substituent is preferably 8 or less.

The repeating unit represented by formula (B) is preferably an acid-decomposable (meth)acrylic acid tertiary ester-based repeating unit (a repeating unit in which Xb represents a hydrogen atom or a methyl group and L represents a —CO— group), an acid-decomposable hydroxystyrene tertiary alkyl ether-based repeating unit (a repeating unit in which Xb represents a hydrogen atom or a methyl group and L represents a phenyl group), or an acid-decomposable styrenecarboxylic acid tertiary ester-based repeating unit (a repeating unit in which Xb represents a hydrogen atom or a methyl group and L represents an -Rt-CO— group (Rt is an aromatic group)).

The content of the repeating unit having the acid-decomposable group including an unsaturated bond with respect to the total amount of the repeating units in the resin (A) is preferably 15% by mole or more, more preferably 20% by mole or more, and still more preferably 30% by mole or more. The upper limit of the content of the repeating unit with respect to the total amount of the repeating units in the resin (A) is preferably 80% by mole or less, more preferably 70% by mole or less, and particularly preferably 60% by mole or less.

Specific example of the repeating unit having the acid-decomposable group including an unsaturated bond are shown below, but the invention is not limited thereto. In the formulas below, Xb and L₁ each represent any of the above-described substituents and linking groups, and Ar represents an aromatic group. R represents a substituent such as a hydrogen atom, an alkyl group, a cycloalkyl group, an aryl group, an aralkyl group, an alkenyl group, a hydroxy group, an alkoxy group, an acyloxy group, a cyano group, a nitro group, an amino group, a halogen atom, an ester group (—OCOR″′ or —COOR″′: R″′ represents an alkyl group having 1 to 20 carbon atoms or a fluorinated alkyl group having 1 to 20 carbon atoms), or a carboxy group, and R′ represents a linear or branched alkyl group, a monocyclic or polycyclic cycloalkyl group, an alkenyl group, an alkynyl group, or a monocyclic or polycyclic aryl group. Q represents a heteroatom such as an oxygen atom, a group including a heteroatom such as a carbonyl group, an —SO₂— group, or an —SO₃— group, a vinylidene group, or a combination thereof. 1, n, and m each represent an integer of 0 or more.

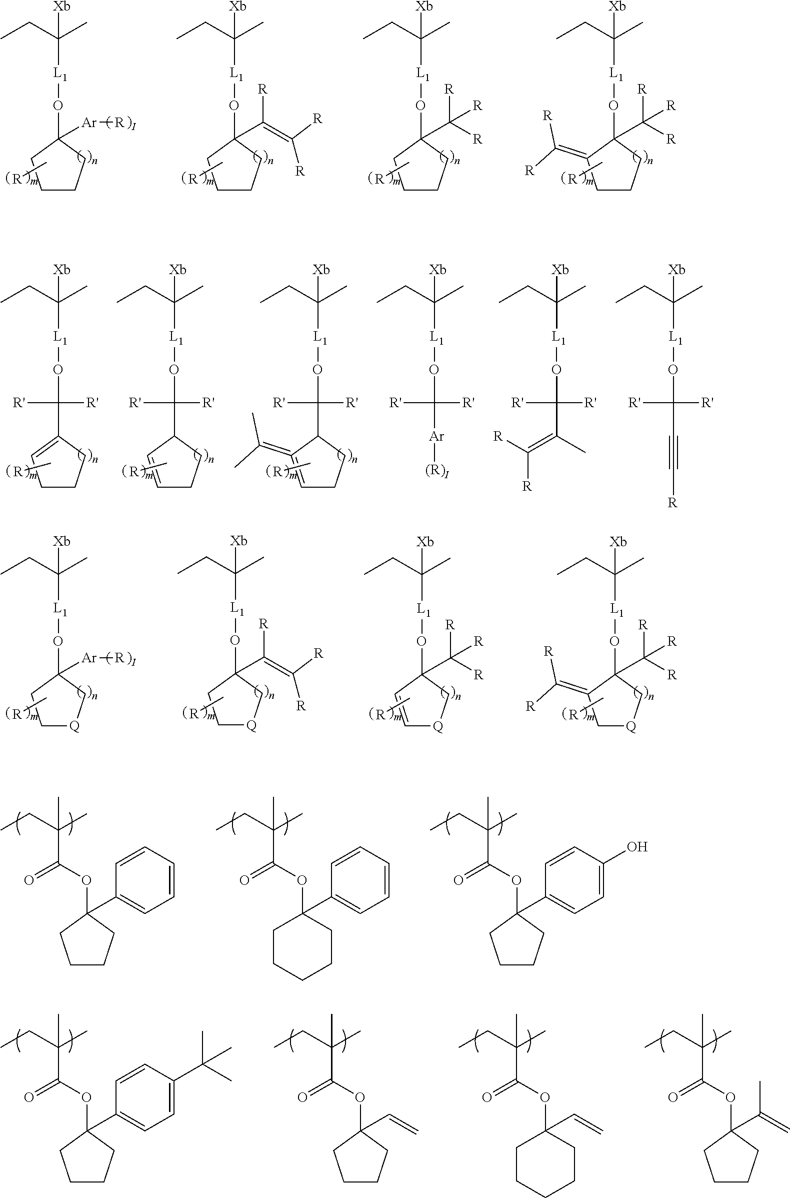

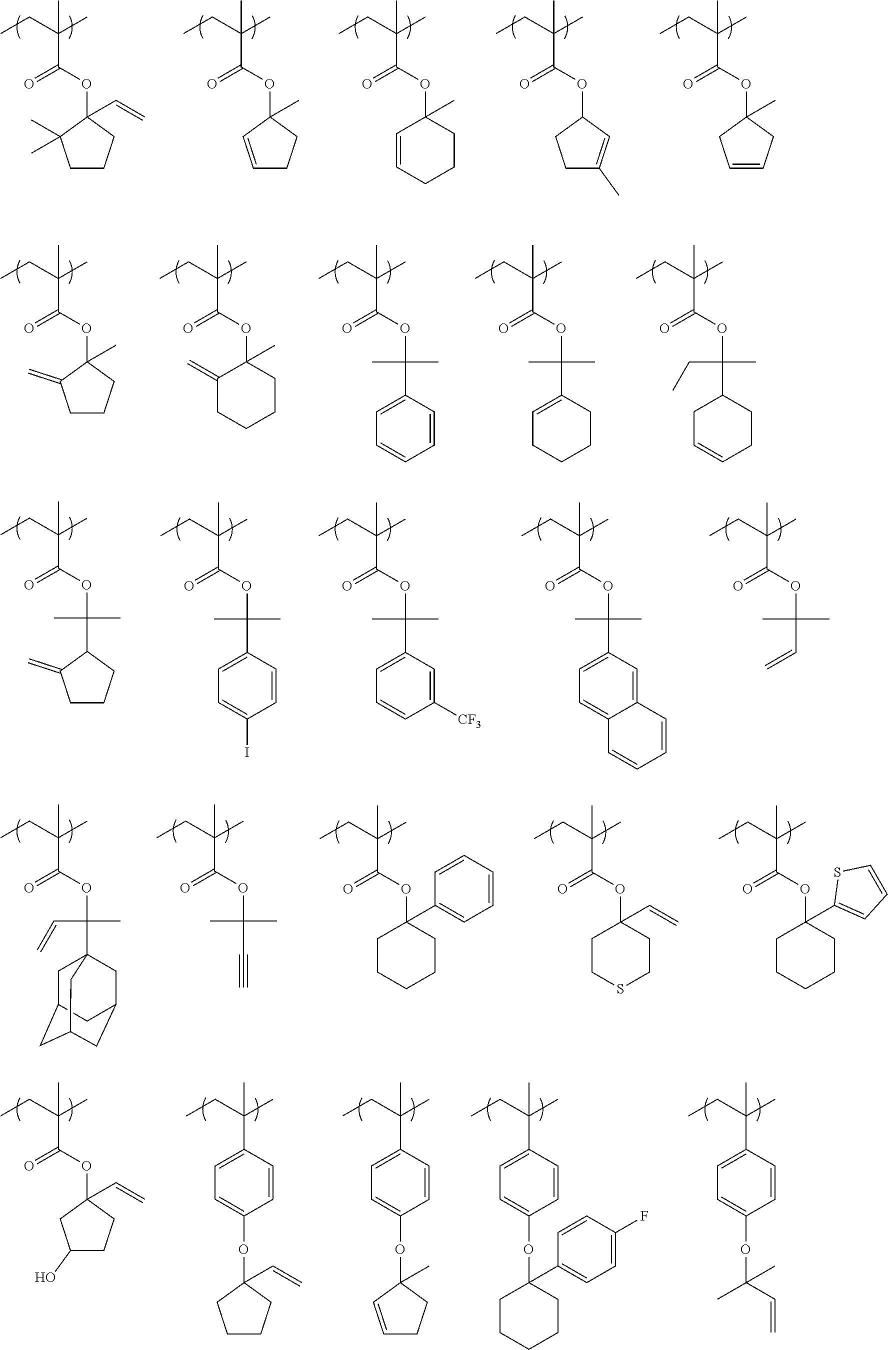

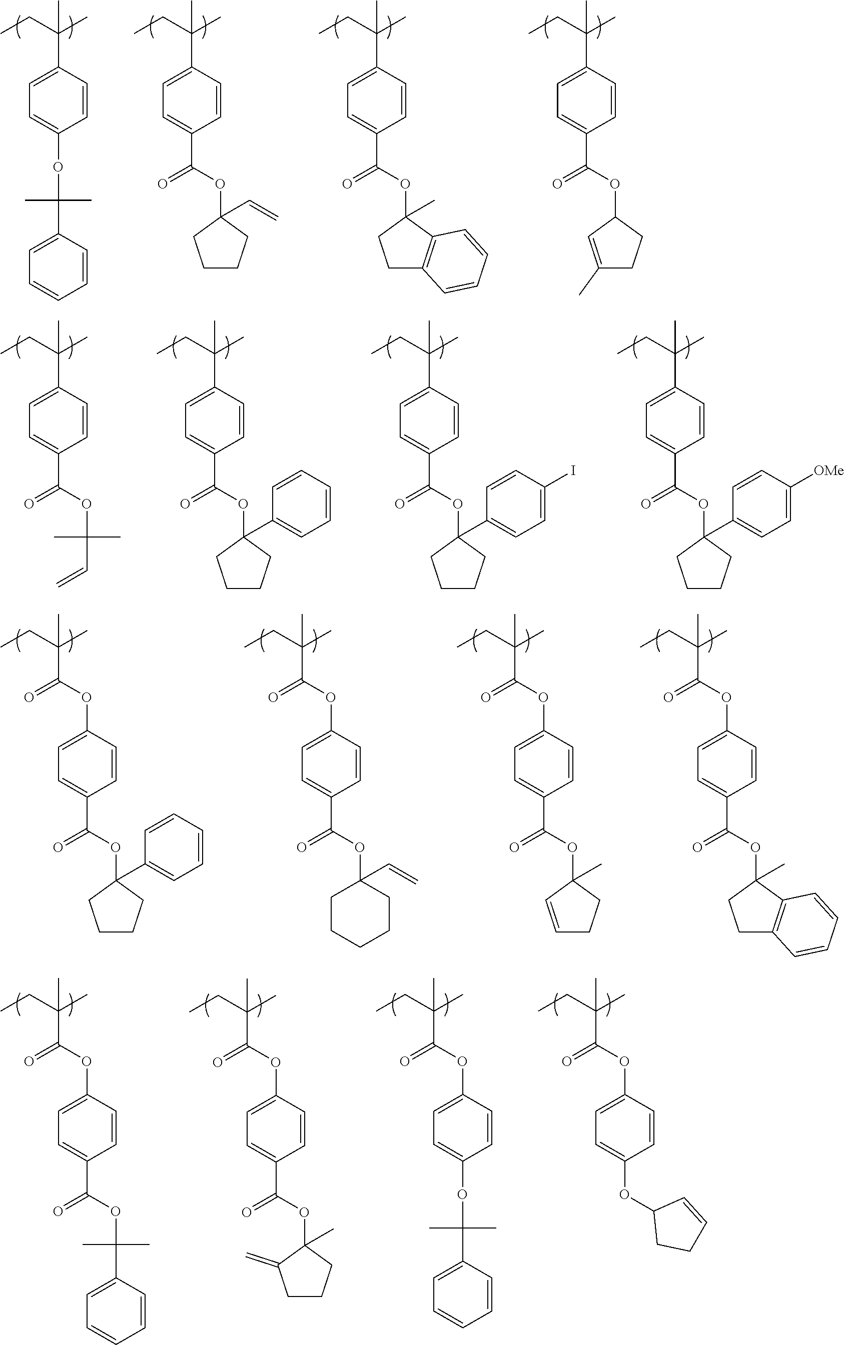

The content of the repeating unit having the acid-decomposable group with respect to the total amount of the repeating units in the resin (A) is preferably 15% by mole or more, more preferably 20% by mole or more, and still more preferably 30% by mole or more. The upper limit of the content of the repeating unit with respect to the total amount of the repeating units in the resin (A) is preferably 90% by mole or less, more preferably 80% by mole or less, still more preferably 70% by mole or less, and particularly preferably 60% by mole or less.

The resin (A) may include at least one repeating unit selected from the following group A and/or at least one repeating unit selected from the following group B.

Group A: The group consisting of the following repeating units (20) to (29).
(20) A repeating unit having an acid group, which will be described later.
(21) A repeating unit having no acid-decomposable group and no acid group but having a fluorine atom, a bromine atom, or an iodine atom, which will be described later.
(22) A repeating unit having a lactone group, a sultone group, or a carbonate group, which will be described later.
(23) A repeating unit having a photoacid generating group, which will be described later.
(24) A repeating unit represented by formula (V-1) or formula (V-2) below, which will be described later.
(25) A repeating unit represented by formula (A), which will be described later.
(26) A repeating unit represented by formula (B), which will be described later.
(27) A repeating unit represented by formula (C), which will be described later.
(28) A repeating unit represented by formula (D), which will be described later. (29) A repeating unit represented by formula (E), which will be described later.

Group B: The group consisting of the following repeating units (30) to (32).
(30) A repeating unit having at least one group selected from the group consisting of lactone groups, sultone groups, carbonate groups, a hydroxy group, a cyano group, and alkali-soluble groups, which will be described later.
(31) A repeating unit having an alicyclic hydrocarbon structure and exhibiting no acid decomposability, which will be described later.
(32) A repeating unit represented by formula (III) and having no hydroxy group and no cyano group, which will be described later.

The resin (A) has preferably an acid group and includes preferably a repeating unit having an acid group as described later. The definition of the acid group will be described later along with preferred modes of the repeating unit having an acid group. When the resin (A) has the acid group, the interaction between the resin (A) and the acid generated from the photoacid generator is enhanced. This results in a further reduction in diffusion of the acid, and a pattern to be formed may have a sharper rectangular cross-sectional shape.

When the resist composition is used as an actinic ray-sensitive or radiation-sensitive resin composition for EUV light, it is preferable that the resin (A) has at least one repeating unit selected from the group A.

When the resist composition is used as an actinic ray-sensitive or radiation-sensitive resin composition for EUV light, it is preferable that the resin (A) includes at least one of a fluorine atom or an iodine atom. When the resin (A) includes both a fluorine atom and an iodine atom, the resin (A) may have one type of repeating unit including both a fluorine atom and an iodine atom or may include two types of repeating units including a repeating unit including a fluorine atom and a repeating unit including an iodine atom.

When the resist composition is used as an actinic ray-sensitive or radiation-sensitive resin composition for EUV light, it is also preferable that the resin (A) has a repeating unit having an aromatic group.

When the resist composition is used as an actinic ray-sensitive or radiation-sensitive resin composition for ArF light, it is preferable that the resin (A) has at least one type of repeating unit selected from the group B.

When the resist composition is used as an actinic ray-sensitive or radiation-sensitive resin composition for ArF light, it is preferable that the resin (A) includes no fluorine atom and no silicon atom.

When the resist composition is used as an actinic ray-sensitive or radiation-sensitive resin composition for ArF light, it is preferable that the resin (A) has no aromatic group.

### (Repeating Unit Having Acid Group)

The resin (A) may have a repeating unit having an acid group.

The acid group is preferably an acid group having a pKa of 13 or less. The acid dissociation constant of the acid group is preferably 13 or less, more preferably 3 to 13, and still more preferably 5 to 10.

When the resin (A) has the acid group having a pKa of 13 or less, no particular limitation is imposed on the content of the acid group in the resin (A), but the content is often 0.2 to 6.0 mmol/g. In particular, the content is preferably 0.8 to 6.0 mmol/g, more preferably 1.2 to 5.0 mmol/g, and still more preferably 1.6 to 4.0 mmol/g. When the content of the acid group is within the above range, development proceeds smoothly, and a pattern to be formed has a good profile, so that high resolution is achieved.

The acid group is preferably, for example, a carboxy group, a phenolic hydroxy group, a fluorinated alcohol group (preferably a hexafluoroisopropanol group), a sulfonic group, a sulfonamido group, or an isopropanol group.

In the hexafluoroisopropanol group, one or more (preferably one to two) fluorine atoms may each be replaced with a group other than a fluorine atom (such as an alkoxycarbonyl group). The acid group is also preferably —C(CF₃)(OH)—CF₂— formed as described above. At least one fluorine atom may be replaced with a group other than a fluorine atom to form a ring including —C(CF₃)(OH)—CF₂—.

Preferably, the repeating unit having the acid group is a repeating unit different from the above-described repeating unit having a structure in which a polar group is protected by a leaving group that leaves by the action of an acid and from a repeating unit having a lactone group, a sultone group, or a carbonate group that is described later.

The repeating unit having the acid group may have a fluorine atom or an iodine atom.

Examples of the repeating unit having the acid group include the following repeating units.

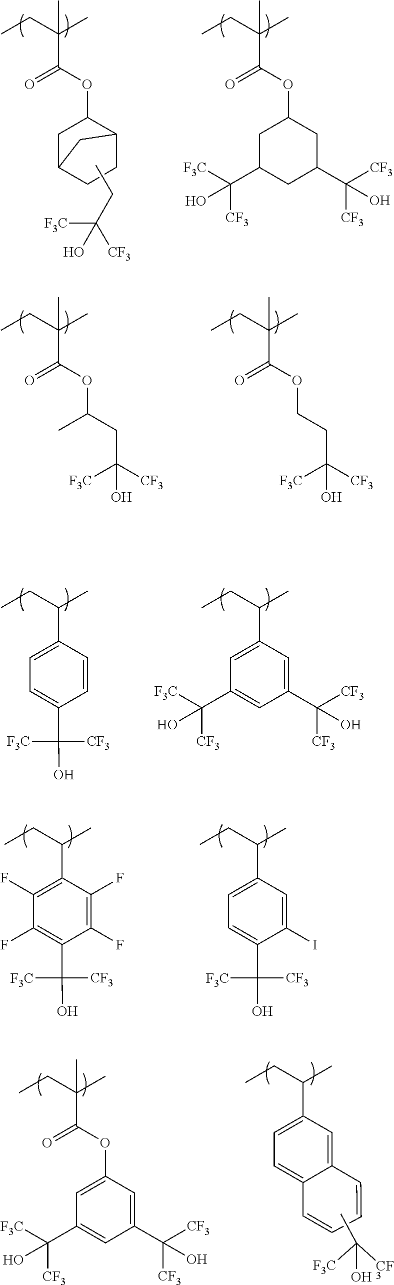

The repeating unit having the acid group is preferably a repeating unit represented by the following formula (1).

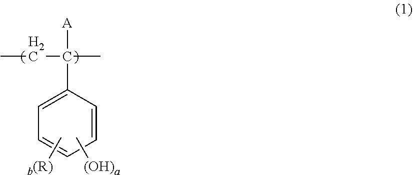

In formula (1), A represents a hydrogen atom, an alkyl group, a cycloalkyl group, a halogen atom, or a cyano group. R represents a halogen atom, an alkyl group, a cycloalkyl group, an aryl group, an alkenyl group, an aralkyl group, an alkoxy group, an alkylcarbonyloxy group, an alkylsulfonyloxy group, an alkyloxycarbonyl group, or an aryloxycarbonyl group. When a plurality of R's are present, they may be the same or different. When a plurality of R's are present, they may together form a ring. R is preferably a hydrogen atom. a represents an integer of from 1 to 3. b represents and integer of 0 to (5-a).

Examples of the repeating unit having the acid group are shown below. In the following formulas, a represents 1 or 2.

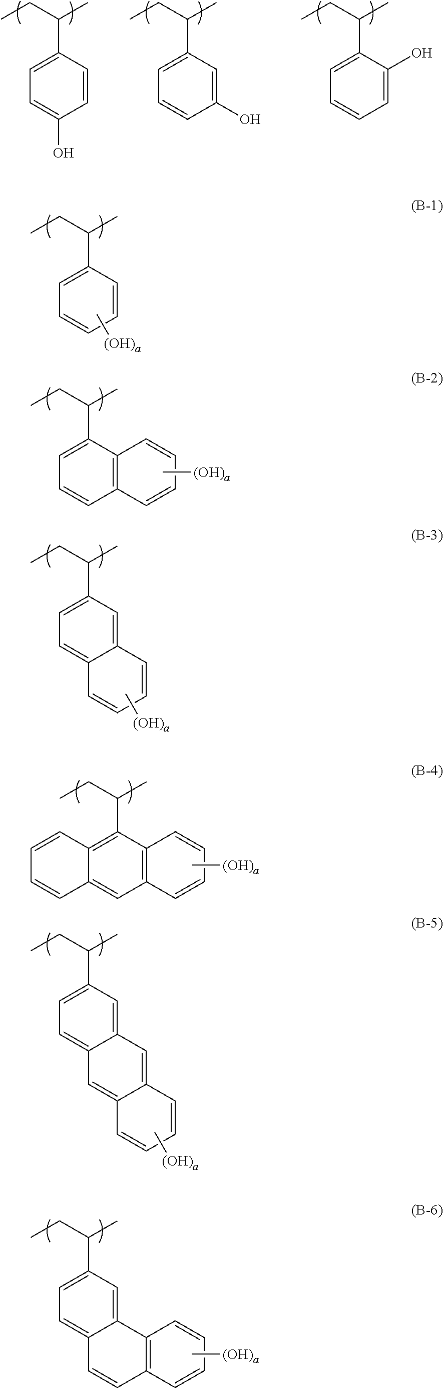

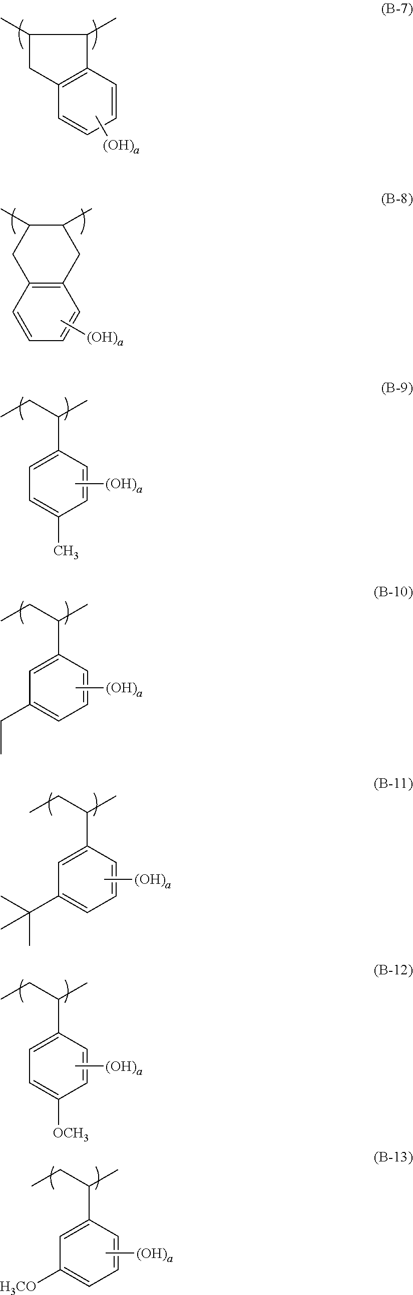

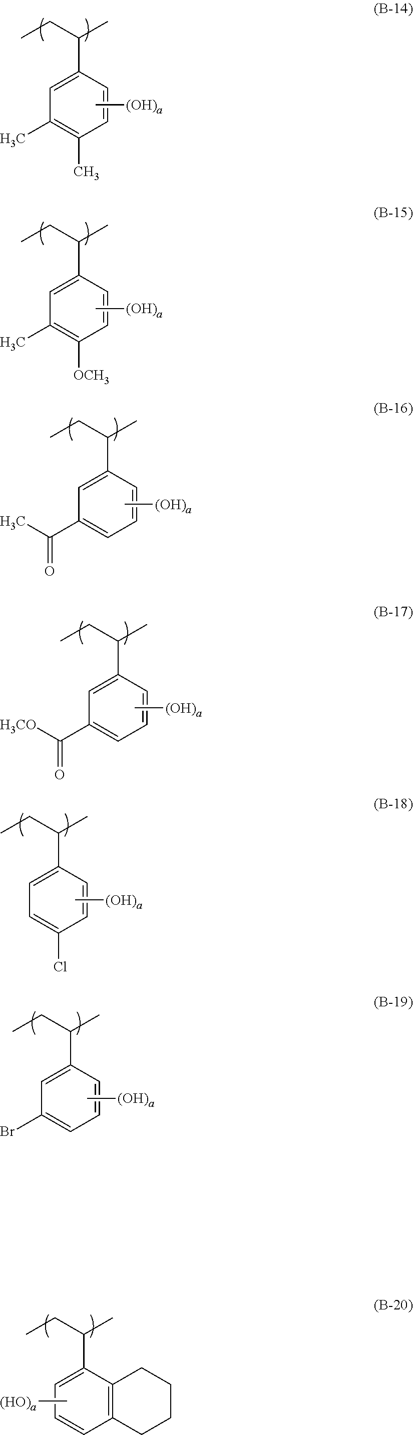

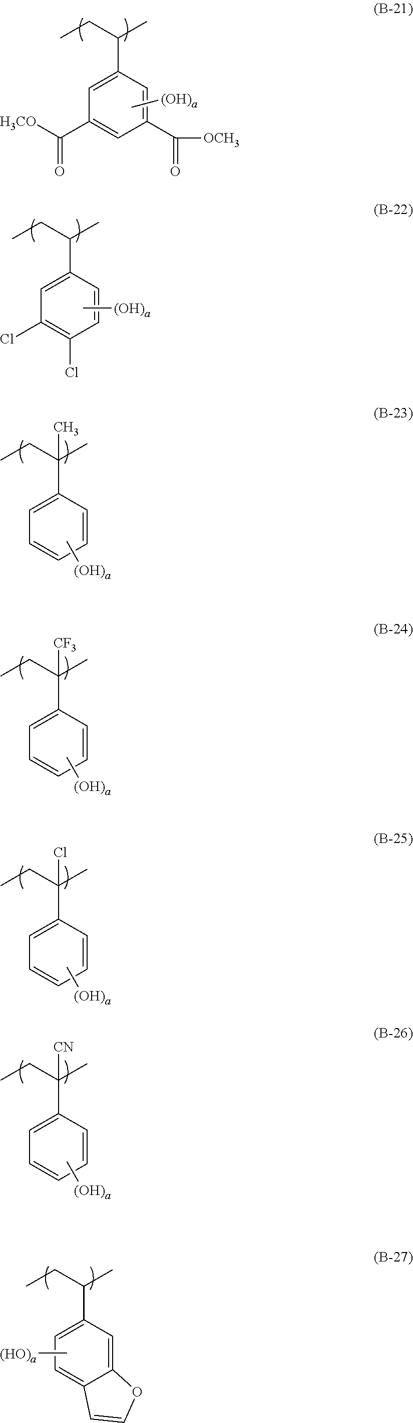

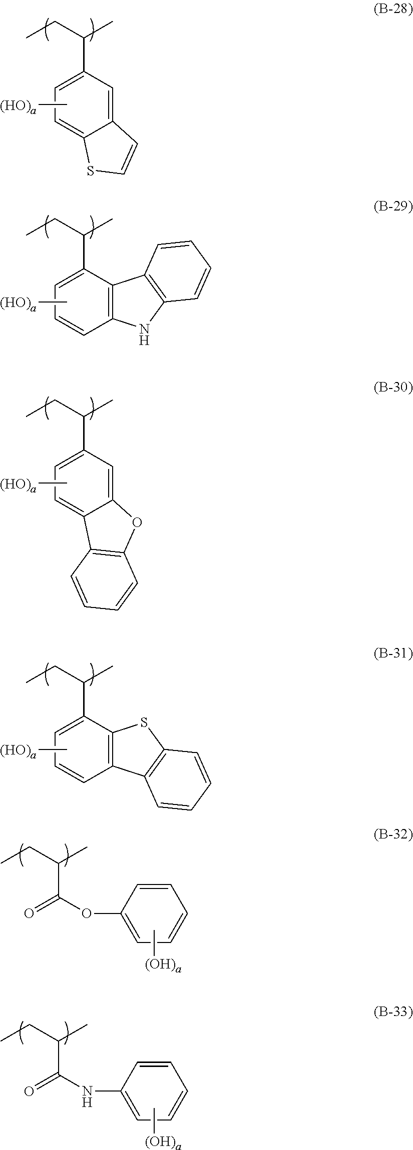

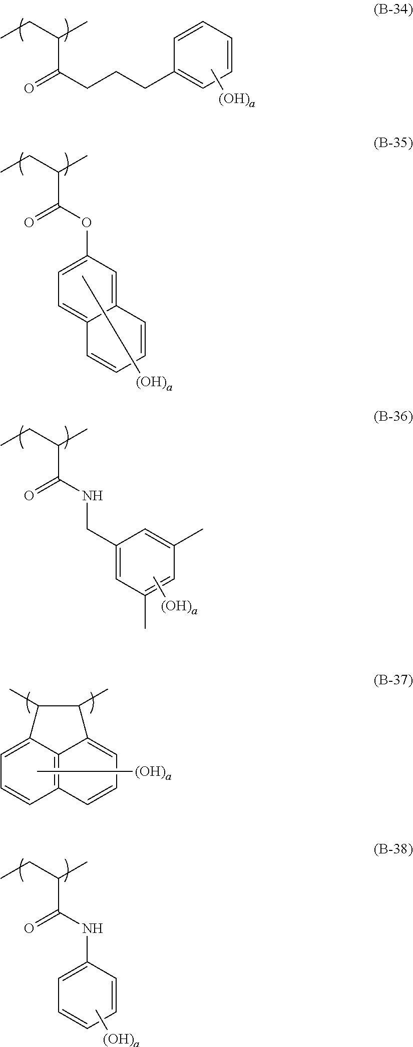

Among the above repeating units, repeating units specifically described below are preferred. In the following formulas, R represents a hydrogen atom or a methyl group, and a represents 2 or 3.

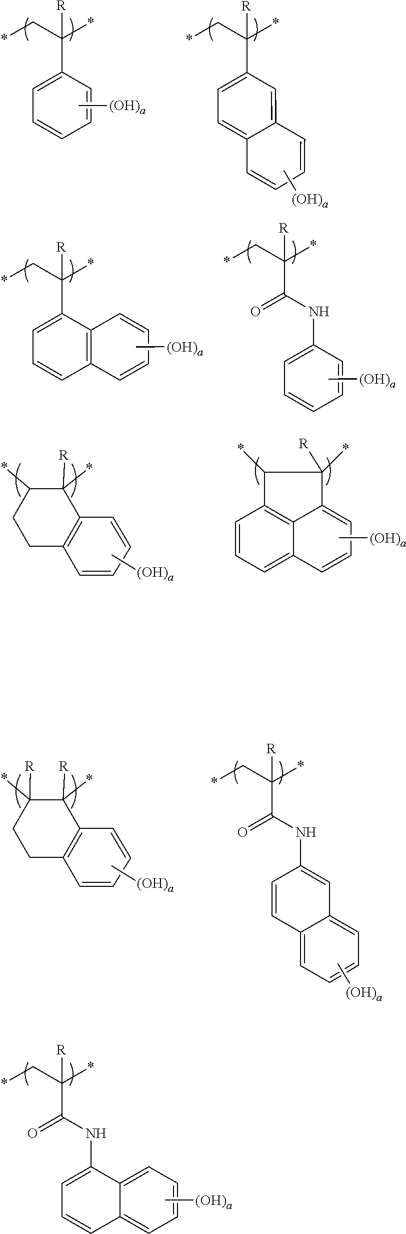

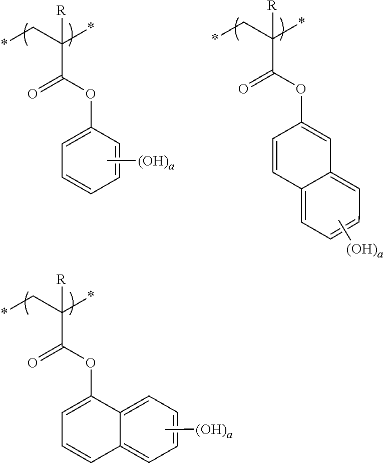

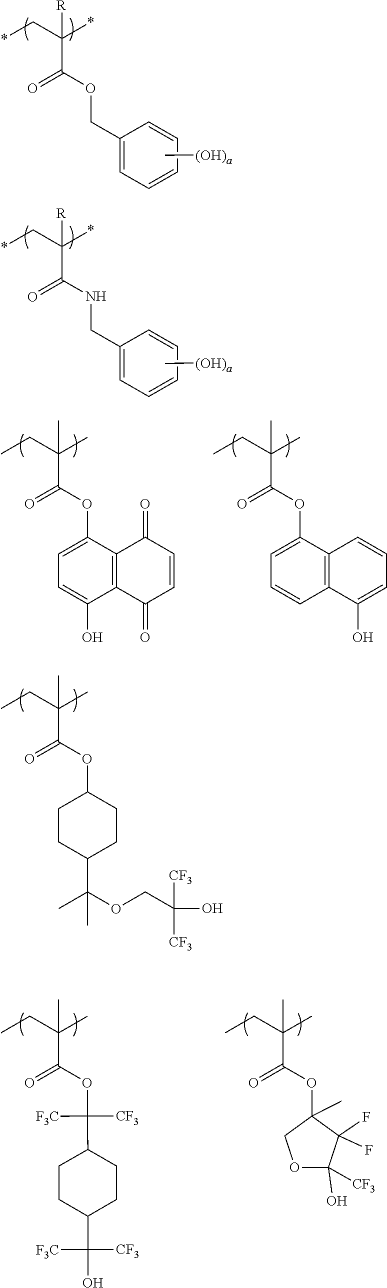

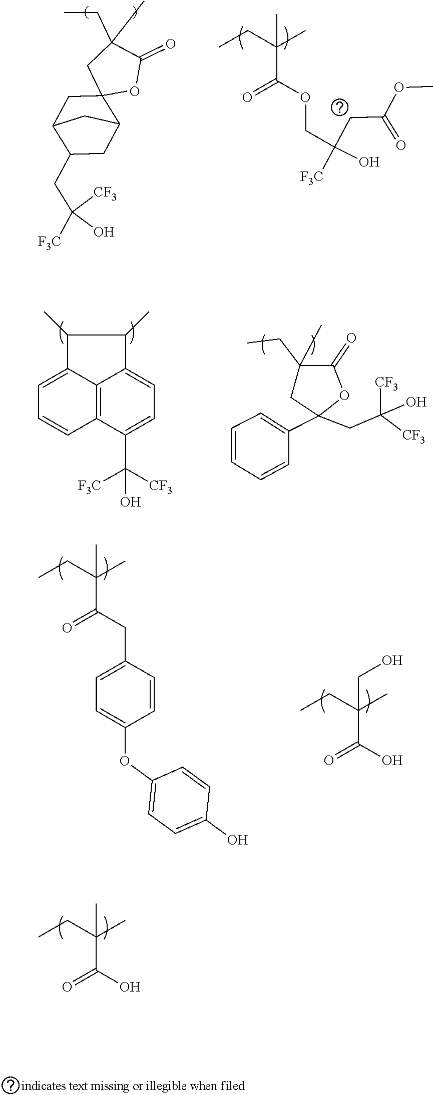

The content of the repeating unit having the acid group with respect to the total amount of the repeating units in the resin (A) is preferably 10% by mole or more and more preferably 15% by mole or more. The upper limit of the content with respect to the total amount of the repeating units in the resin (A) is preferably 70% by mole or less. more preferably 65% by mole or less, and still more preferably 60% by mole or less.

### (Repeating Unit Having No Acid-Decomposable Group and No Acid Group but Having Fluorine Atom, Bromine Atom, or Iodine Atom)

The resin (A) may have, in addition to the above-described <repeating unit having the acid-decomposable group> and the above-described <repeating unit having the acid group>, a repeating unit having no acid-decomposable group and no acid group but having a fluorine atom, a bromine atom, or an iodine atom (this repeating unit is hereinafter referred to also as a unit X). Preferably, the <repeating unit having no acid-decomposable group and no acid group but having a fluorine atom, a bromine atom, or an iodine atom> differs from other types of repeating units belonging to the group A such as the <repeating unit having a lactone group, a sultone group, or a carbonate group> described later and the <repeating unit having a photoacid generating group> described later.

The unit X is preferably a repeating unit represented by formula (C).

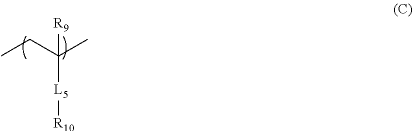

L₅ represents a single bond or an ester group. R₉ represents a hydrogen atom or an alkyl group optionally having a fluorine atom or an iodine atom. R₁₀ represents a hydrogen atom, an alkyl group optionally having a fluorine atom or an iodine atom, a cycloalkyl group optionally having a fluorine atom or an iodine atom, an aryl group optionally having a fluorine atom or an iodine atom, or a combination thereof.

Examples of the repeating unit having a fluorine atom or an iodine atom are shown below.

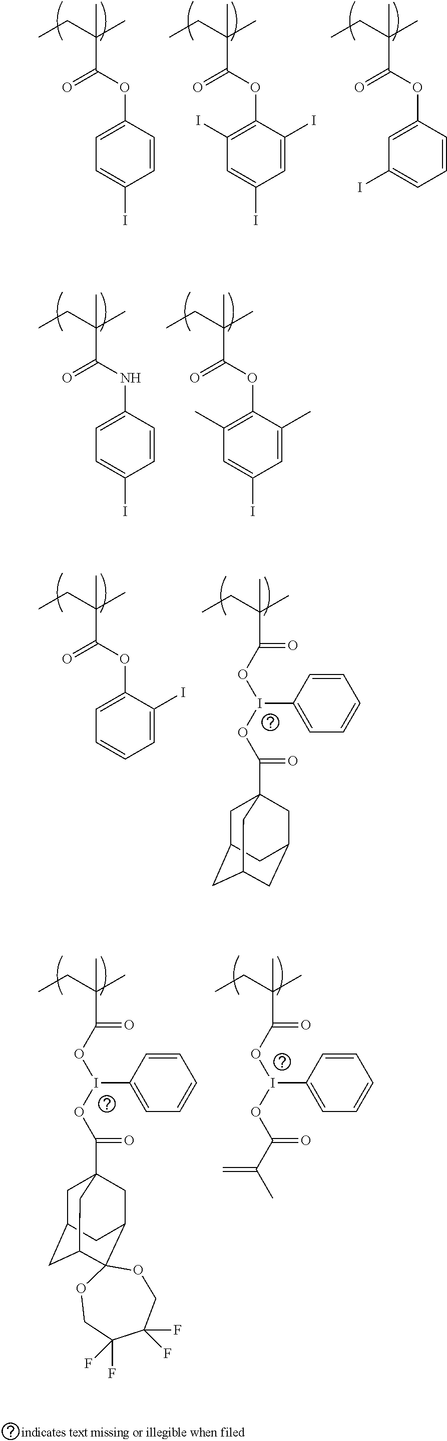

The content of the unit X with respect to the total amount of the repeating units in the resin (A) is preferably 0% by mole or more, more preferably 5% by mole or more, and still more preferably 10% by mole or more. The upper limit of the content of the unit X with respect to the total amount of the repeating units in the resin (A) is preferably 50% by mole or less, more preferably 45% by mole or less, and still more preferably 40% by mole or less.

Among the repeating units in the resin (A), the total amount of repeating units including at least one of a fluorine atom, a bromine atom, or an iodine atom with respect to the total amount of the repeating units in the resin (A) is preferably 10% by mole or more, more preferably 20% by mole or more, still more preferably 30% by mole or more, and particularly preferably 40% by mole or more. No particular limitation is imposed on the upper limit of the total amount, but the amount with respect to the total amount of the repeating units in the resin (A) is, for example, 100% by mole or less.

Examples of the repeating units including at least one of a fluorine atom, a bromine atom, or an iodine atom include: a repeating unit having a fluorine atom, a bromine atom, or an iodine atom and having the acid-decomposable group; a repeating unit having a fluorine atom, a bromine atom, or an iodine atom and having the acid group; and a repeating unit having a fluorine atom, a bromine atom, or an iodine atom.

### (Repeating Unit Having Lactone Group, Sultone Group, or Carbonate Group)

The resin (A) may have a repeating unit having at least one selected from the group consisting of lactone groups, sultone groups, and carbonate groups (this repeating unit is hereafter referred to also as a “unit Y”).

It is also preferable that the unit Y does not have a hydroxy group and an acid group such as a hexafluoropropanol group.

The lactone or sultone group may be any lactone or sultone group so long as it has a lactone or sultone structure. The lactone or sultone structure is preferably a 5- to 7-membered lactone or sultone structure. In particular, a 5- to 7-membered lactone structure with another ring structure fused thereto to form a bicyclo or spiro structure or a 5- to 7-membered sultone structure with another ring structure fused thereto to form a bicyclo or spiro structure is more preferred.

Preferably, the resin (A) has a repeating unit having a lactone or sultone group formed by removing at least one hydrogen atom from a ring member atom of a lactone structure represented by any of the following formulas (LC1-1) to (LC1-21) or a sultone structure represented by any of the following formulas (SL1-1) to (SL1-3).

The lactone or sultone group may be bonded directly to the main chain. For example, a ring member atom of the lactone or sultone group may be included in the main chain of the resin (A).

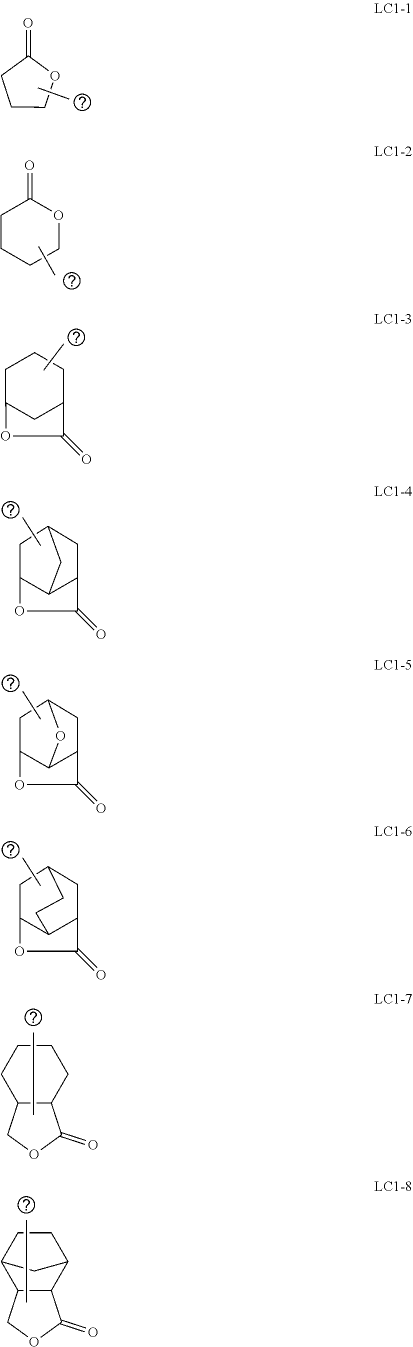

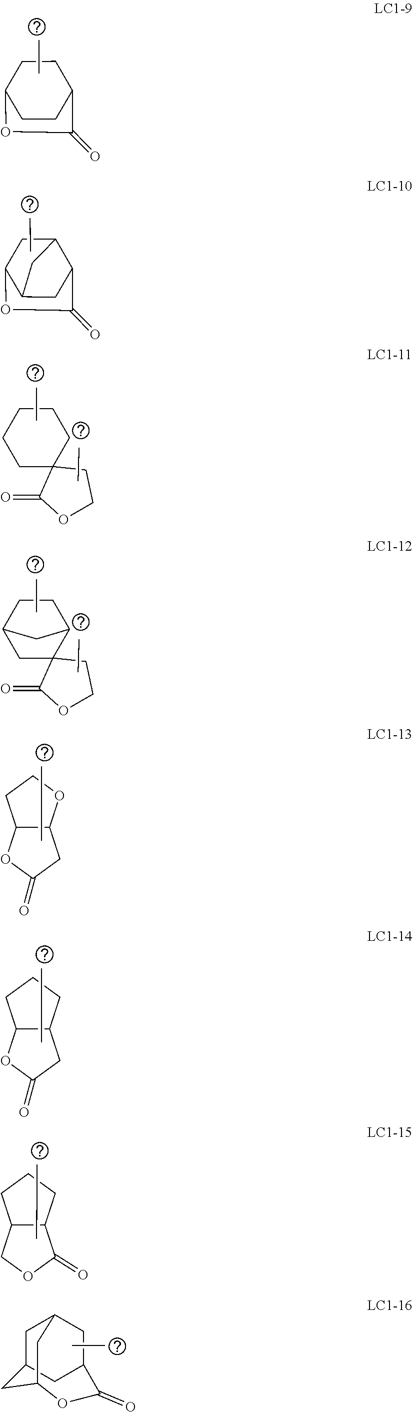

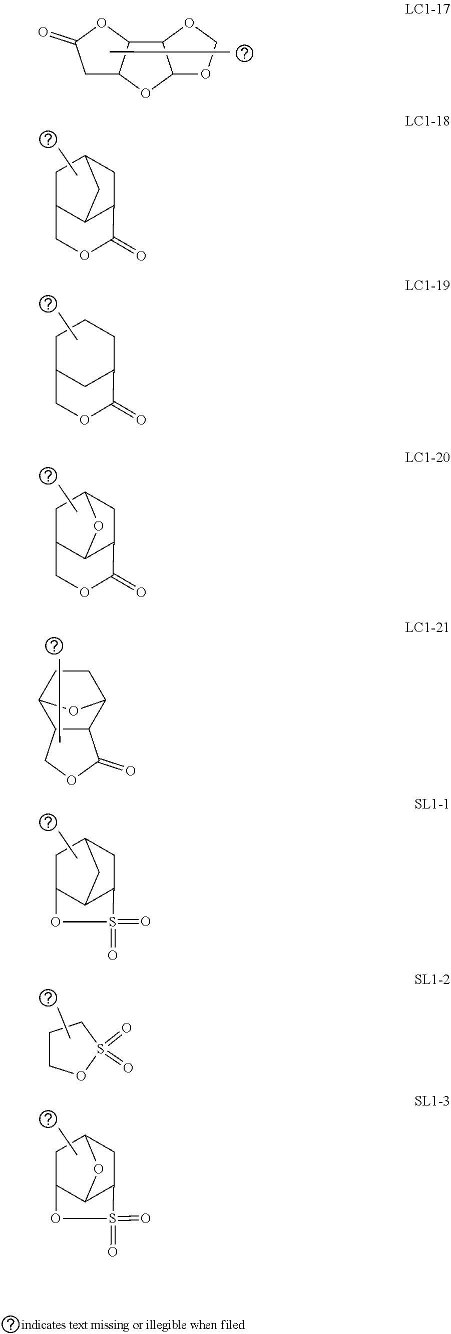

Each of the lactone and sultone structures may have a substituent (Rb₂). Preferred examples of the substituent (R_{b}2) include alkyl groups having 1 to 8 carbon atoms, cycloalkyl groups having 4 to 7 carbon atoms, alkoxy groups having 1 to 8 carbon atoms, alkoxycarbonyl groups having 1 to 8 carbon atoms, a carboxy group, halogen atoms, a cyano group, and acid-decomposable groups. n₂ represents an integer of from 0 to 4. A plurality of Rb₂'s present when n₂ is 2 or more may be different from each other, and the plurality of Rb₂'s present may be bonded together to form a ring.

Examples of the repeating unit having a group including the lactone structure represented by any of formulas (LC1-1) to (LC1-21) or the sultone structure represented by any of formulas (SL1-1) to (SL1-3) include a repeating unit represented by the following formula (AI).

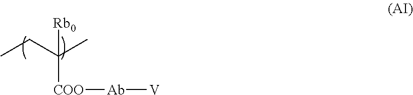

In formula (AI), Rb₀ represents a hydrogen atom, a halogen atom, or an alkyl group having 1 to 4 carbon atoms. The alkyl group represented by Rb₀ may have a substituent, and preferred examples of the substituent include a hydroxy group and halogen atoms.

Examples of the halogen atom represented by Rb₀ include a fluorine atom, a chlorine atom, a bromine atom, and an iodine atom. Rb₀ is preferably a hydrogen atom or a methyl group.

Ab represents a single bond, an alkylene group, a divalent linking group having a monocyclic or polycyclic alicyclic hydrocarbon structure, an ether group, an ester group, a carbonyl group, a carboxy group, or a divalent group formed by combining any of the above groups. In particular, Ab is preferably a single bond or a linking group represented by -Ab₁-CO₂—. Ab₁ is a linear or branched alkylene group or a monocyclic or polycyclic cycloalkylene group and is preferably a methylene group, an ethylene group, a cyclohexylene group, an adamantylene group, or a norbornylene group.

V represents a group formed by removing one hydrogen atom from a ring member atom in the lactone structure represented by any of formulas (LC1-1) to (LC1-21) or a group formed by removing one hydrogen atom from a ring member atom in the sultone structure represented by any of formulas (SL1-1) to (SL1-3).

When the repeating unit having the lactone or sultone group has optical isomers, any of the optical isomers may be used. One optical isomer may be used alone, or a mixture of a plurality of optical isomers may be used. When one optical isomer is mainly used, the optical purity (ee) thereof is preferably 90% or more and more preferably 95% or more.

The carbonate group is preferably a cyclic carbonate group.

The repeating unit having a cyclic carbonate group is preferably a repeating unit represented by the following formula (A-1).

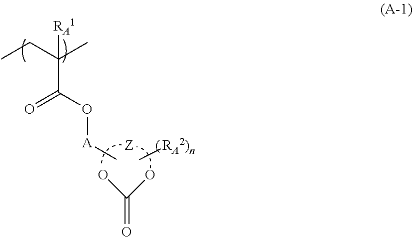

In formula (A-1), R_{A}¹ represents a hydrogen atom, a halogen atom, or a monovalent organic group (preferably a methyl group). n represents an integer of 0 or more. R_{A}² represents a substituent. A plurality of R_{A}²'s present when n is 2 or more may be the same or different. A represents a single bond or a divalent linking group. The divalent linking group is preferably an alkylene group, a divalent linking group having a monocyclic or polycyclic alicyclic hydrocarbon structure, an ether group, an ester group, a carbonyl group, a carboxy group, or a divalent group formed by combining any of them. Z represents an atomic group forming a monocyclic or polycyclic ring together with a group represented by —O—CO—O— in formula (A-1).

Examples of the unit Y are shown below. In these formulas, Rx represents a hydrogen atom, —CH₃, —CH₂OH, or —CF₃.

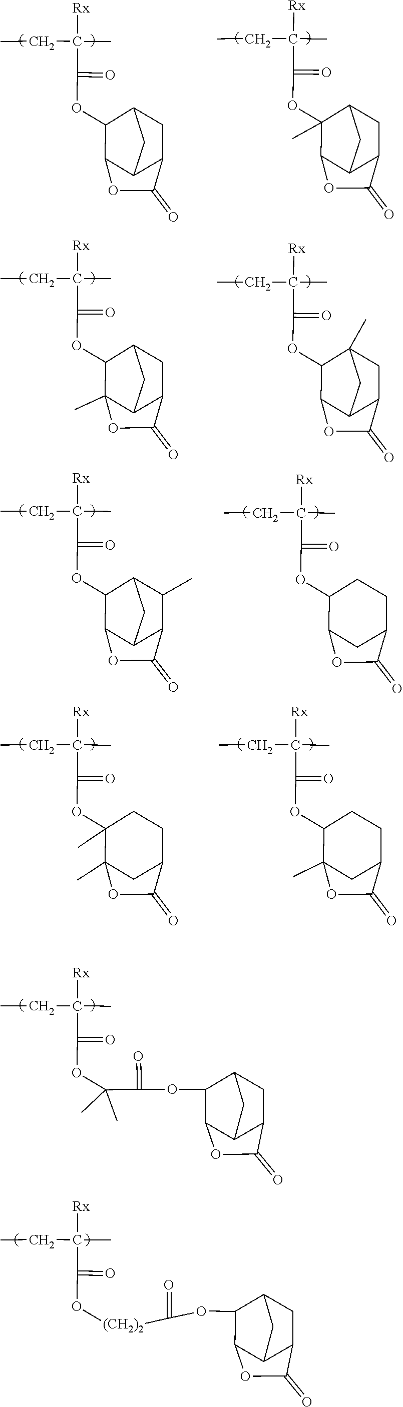

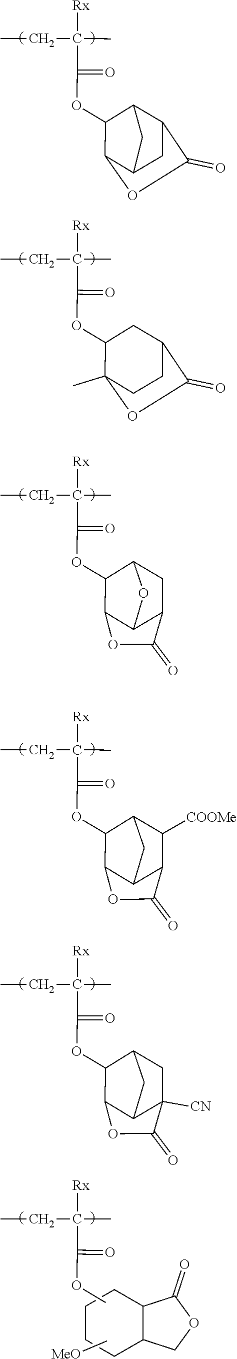

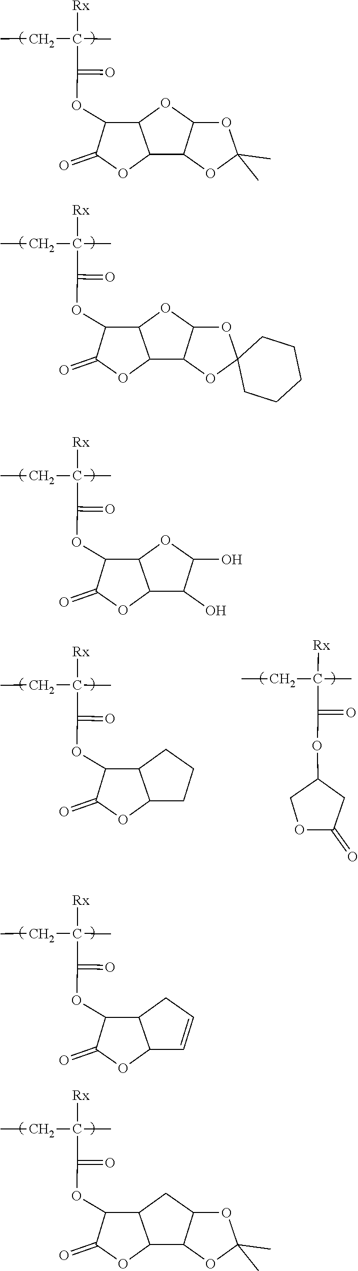

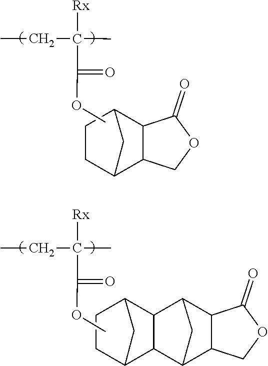

(In the formulas, Rx is H, CH₃, CH₂OH, or CF₃.)

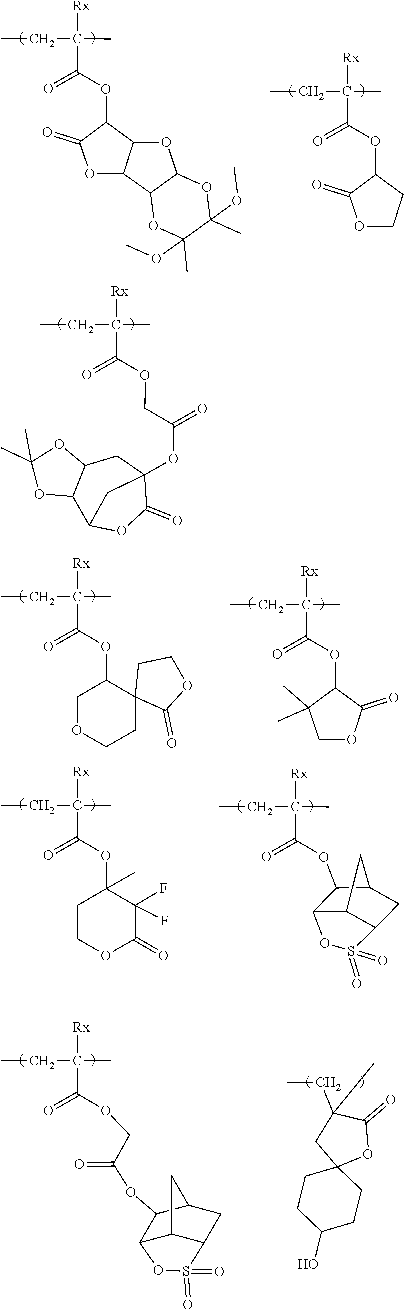

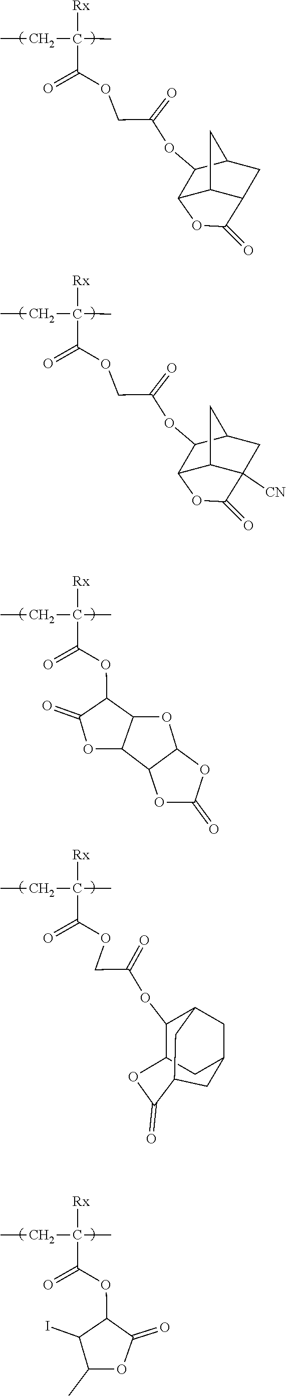

(In the formulas, Rx is H, CH₃, CH₂OH, or CF₃.)

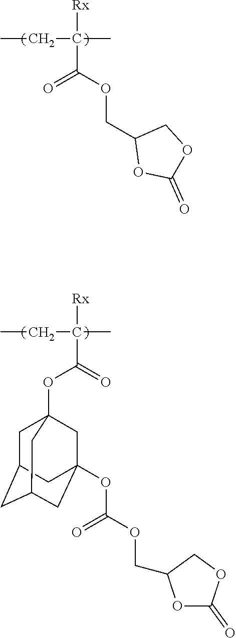

The content of the unit Y with respect to the total amount of the repeating units in the resin (A) is preferably 1% by mole or more and more preferably 10% by mole or more. The upper limit of the content of the unit Y with respect to the total amount of the repeating units in the resin (A) is preferably 85% by mole or less, more preferably 80% by mole or less, still more preferably 70% by mole or less, and particularly preferably 60% by mole or less.

### (Repeating Unit Having Photoacid Generating Group)

The resin (A) may include a repeating unit that is different from those described above and has a group that generates an acid when irradiated with actinic rays or radiation (this group is hereinafter referred to also as a “photoacid generating group”).

Examples of the repeating unit having the photoacid generating group include a repeating unit represented by formula (4).

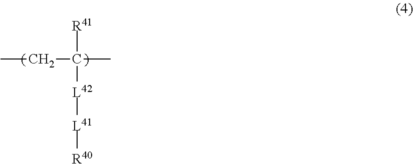

R⁴¹ represents a hydrogen atom or a methyl group. L⁴¹ represent a single bond or a divalent linking group. L⁴² represents a divalent linking group. R^{4′} represents a structural moiety that is decomposed when irradiated with actinic rays or radiation and thereby generates an acid on a side chain.

Examples of the repeating unit having the photoacid generating group are shown below.

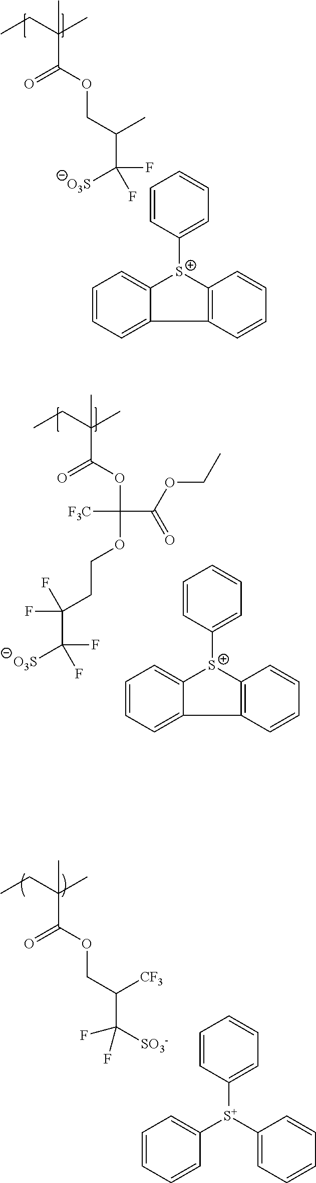

Other examples of the repeating unit represented by formula (4) include repeating units described in paragraphs [0094] to [0105] of JP2014-041327A and repeating units described in paragraph [0094] of WO2018/193954A.

The content of the repeating unit having the photoacid generating group with respect to the total amount of the repeating units in the resin (A) is preferably 1% by mole or more and more preferably 5% by mole or more. The upper limit of the content with respect to the total amount of the repeating units in the resin (A) is preferably 40% by mole or less, more preferably 35% by mole or less, and still more preferably 30% by mole or less.

### (Repeating Unit Represented by Formula (V-1) or Formula (V-2))

The resin (A) may have a repeating unit represented by formula (V-1) or (V-2) below.

Preferably, the repeating unit represented by the following formula (V-1) or (V-2) differs from the repeating units described above.

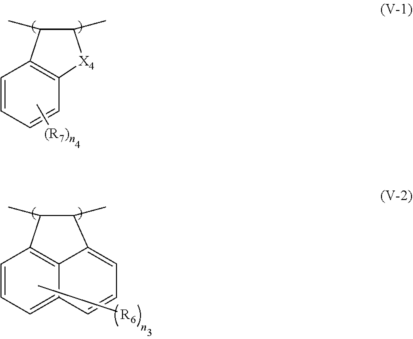

In these formulas, R₆ and R₇ each independently represent a hydrogen atom, a hydroxy group, an alkyl group, an alkoxy group, an acyloxy group, a cyano group, a nitro group, an amino group, a halogen atom, an ester group (—OCOR or —COOR: R represents an alkyl group having 1 to 6 carbon atoms or a fluorinated alkyl group having 1 to 6 carbon atoms), or a carboxy group. The alkyl group is preferably a linear, branched, or cyclic alkyl group having 1 to 10 carbon atoms.

n₃ represents an integer of from 0 to 6.

n₄ represents an integer of from 0 to 4.

X₄ is a methylene group, an oxygen atom, or a sulfur atom.

Examples of the repeating unit represented by formula (V-1) or (V-2) are shown below.

Examples of the repeating unit represented by formula (V-1) or (V-2) include repeating units described in paragraph [0100] of WO2018/193954A.

### (Repeating Unit for Reducing Mobility of Main Chain)

The higher the glass transition temperature (Tg) of the resin (A), the better because excessive diffusion of the acid generated or pattern collapse during development can be prevented. The Tg is preferably higher than 90° C., more preferably higher than 100° C., still more preferably higher than 110° C., and particularly preferably higher than 125° C. The Tg is preferably 400° C. or lower and more preferably 350° C. or lower because the rate of dissolution in a developer is high.

In the present specification, the glass transition temperature (Tg) of a polymer such as the resin (A) (hereinafter referred to as the “Tg of a repeating unit”) is computed by the following method. First, the Tg of each of the homopolymers formed from the respective repeating units included in the polymer is computed by the Bicerano method. Next, the mass ratios (%) of the repeating units with respect to the total mass of the repeating units in the polymer are computed. Next, the Tg of each repeating unit at the corresponding mass ratio is computed using the Fox formula (described, for example, in Materials Letters 62 (2008) 3152), and the computed Tg's are summed to obtain the Tg (° C.) of the polymer.

The Bicerano method is described in Prediction of polymer properties, Marcel Dekker Inc, New York (1993). The computation of Tg by the Bicerano method can be performed using software for estimating physical properties of a polymer, MDL Polymer (MDL Information Systems, Inc.).

To increase the Tg of the resin (A) (to increase the Tg to preferably higher than 90° C.), it is preferable to reduce the mobility of the main chain of the resin (A). Examples of a method for reducing the mobility of the main chain of the resin (A) include methods (a) to (e) described below.
(a) Introduction of a bulky substituent into the main chain.
(b) Introduction of a plurality of substituents into the main chain.
(c) Introduction of a substituent that induces the interaction between molecules of the resin (A) into the vicinities of their main chains.
(d) Formation of the main chain having a ring structure.
(e) Linkage of a ring structure to the main chain Preferably, the resin (A) has a repeating unit whose homopolymer has a Tg of 130° C. or higher.

No particular limitation is imposed on the type of repeating unit whose homopolymer has a Tg of 130° C. or higher, and any repeating unit can be used so long as the Tg of the homopolymer computed by the Bicerano method is 130° C. or higher. With repeating units represented by formulas (A) to (E) described below, homopolymers formed from the repeating units can have a Tg of 130° C. or higher, but this depends on the types of functional groups in the repeating units.

One specific example of means for achieving the method (a) is a method in which the repeating unit represented by formula (A) is introduced into the resin (A).

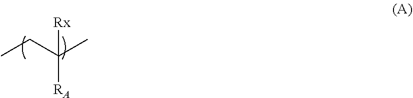

In formula (A), R_{A} represents a group including a polycyclic structure. Rx represents a hydrogen atom, a methyl group, or an ethyl group. The group including the polycyclic structure is a group including a plurality of ring structures, and the plurality of ring structures may or may not be fused.

Specific examples of the repeating unit represented by formula (A) include those described in paragraphs [0107] to [0119] of WO2018/193954A.

One specific example of means for achieving the method (b) is a method in which the repeating unit represented by formula (B) is introduced into the resin (A).

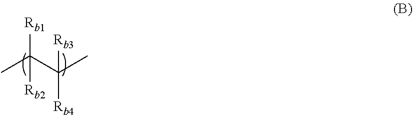

In formula (B), R_{b1} to R_{b4} each independently represent a hydrogen atom or an organic group, and at least two selected from the group consisting of R_{b1} to R_{b4} each represent an organic group.

When at least one of the organic groups is a group whose ring structure is linked directly to the main chain of the repeating unit, no particular limitation is imposed on the types of other organic groups.

When each of the organic groups is not a group whose ring structure is linked directly to the main chain of the repeating unit, at least two of the organic groups are each a substituent in which the number of constituent atoms excluding hydrogen atoms is 3 or more.

Specific examples of the repeating unit represented by formula (B) include those described in paragraphs [0113] to [0115] of WO2018/193954A.

One specific example of means for achieving the method (c) is a method in which the repeating unit represented by formula (C) is introduced into the resin (A).

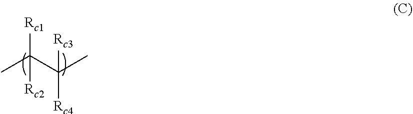

In formula (C), R_{c1} to R_{c4} each independently represent a hydrogen atom or an organic group, and at least one of R_{c1}, R_{c2}, R_{c3}, or R_{c4} is a group including a hydrogen-bonding hydrogen atom at a position within 3 atoms from a carbon atom in the main chain. In particular, it is preferable that the hydrogen-bonding hydrogen atom is present at a position within two atoms (at a position closer to the main chain) in order to induce the interaction between the main chains of molecules of the resin (A).

Specific examples of the repeating unit represented by formula (C) include those described in paragraphs [0119] to [0121] of WO2018/193954A.

One specific example of means for achieving the method (d) is a method in which the repeating unit represented by formula (D) is introduced into the resin (A).

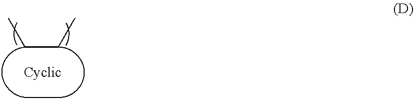

In formula D, “Cyclic” represents a group having a ring structure forming the main chain. No particular limitation is imposed on the number of atoms forming the ring.

Specific examples of the repeating unit represented by formula (D) include those described in paragraphs [0126] to [0127] of WO2018/193954A.

One specific example of means for achieving the method (e) is a method in which the repeating unit represented by formula (E) is introduced into the resin (A).

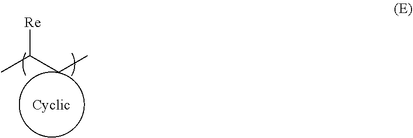

In formula (E), Re's each independently represent a hydrogen atom or an organic group. Examples of the organic group include alkyl groups, cycloalkyl groups, aryl groups, aralkyl groups, and alkenyl groups, each of which may have a substituent.

“Cyclic” is a cyclic group including a carbon atom included in the main chain. No particular limitation is imposed on the number of atoms included in the cyclic group.

Specific examples of the repeating unit represented by formula (E) include those described in paragraphs [0131] to [0133] of WO2018/193954A.

(Repeating Unit Having at Least One Group Selected from Group Consisting of Lactone Groups, Sultone Groups, Carbonate Groups, Hydroxy Group, Cyano Group, and Alkali-Soluble Groups)

The resin (A) may have a repeating unit having at least one group selected from the group consisting of lactone groups, sultone groups, carbonate groups, a hydroxy group, a cyano group, and alkali-soluble groups.

The repeating unit having a lactone group, a sultone group, or a carbonate group and included in the resin (A) may be any of the repeating units described above for the <repeating unit having a lactone group, a sultone group, or a carbonate group>. A preferred content of the repeating unit is also as described above for the <repeating unit having a lactone group, a sultone group, or a carbonate group>.

The resin (A) may have a repeating unit having a hydroxy group or a cyano group. In this case, the adhesiveness to a substrate and the affinity for a developer are improved.

The repeating unit having a hydroxy group or a cyano group is preferably a repeating unit having an alicyclic hydrocarbon structure substituted with a hydroxy group or a cyano group.

Preferably, the repeating unit having a hydroxy group or a cyano group has no acid-decomposable group. Examples of the repeating unit having a hydroxy group or a cyano group include those described in paragraphs [0081] to [0084] of JP2014-098921A.

The resin (A) may have a repeating unit having an alkali-soluble group.

Examples of the alkali-soluble group include a carboxy group, a sulfonamido group, a sulfonylimido group, a bissulfonylimido group, and aliphatic alcohols substituted with an electron-withdrawing group at the α-position (e.g., a hexafluoroisopropanol group), and the alkali-soluble group is preferably a carboxy group. When the resin (A) includes the repeating unit having an alkali-soluble group, resolution in contact hole applications is increased. Examples of the repeating unit having an alkali-soluble group include those described in paragraphs [0085] and [0086] of JP2014-098921A.

### (Repeating Unit Having Alicyclic Hydrocarbon Structure and Exhibiting No Acid Decomposability)

The resin (A) may have a repeating unit having an alicyclic hydrocarbon structure and exhibiting no acid decomposability. In this case, elution of a low-molecular weight component from the resist film to an immersion liquid during liquid immersion exposure can be reduced. Examples of such a repeating unit include repeating units derived from 1-adamantyl (meth)acrylate, diamantyl (meth)acrylate, tricyclodecanyl (meth)acrylate, and cyclohexyl (meth)acrylate.

### (Repeating Unit Represented by Formula (III) and Having No Hydroxy Group and No Cyano Group)

The resin (A) may have a repeating unit represented by formula (III) and having no hydroxy group and no cyano group.

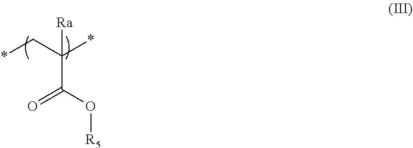

In formula (III), R₅ represents a hydrocarbon group having at least one ring structure and having no hydroxy group and no cyano group.

Ra represents a hydrogen atom, an alkyl group, or a —CH₂—O—Ra₂ group. In this formula, Ra₂ represents a hydrogen atom, an alkyl group, or an acyl group.

Examples of the repeating unit represented by formula (III) and having no hydroxy group and no cyano group include those described in paragraphs [0087] to [0094] of JP2014-098921A.

### (Additional Repeating Units)

The resin (A) may further have a repeating unit other than the repeating units described above.

For example, the resin (A) may have a repeating unit selected from the group consisting of a repeating unit having an oxathiane ring group, a repeating unit having an oxazolone ring group, a repeating unit having a dioxane ring group, and a repeating unit having a hydantoin ring group.

Examples of such a repeating unit are shown below.

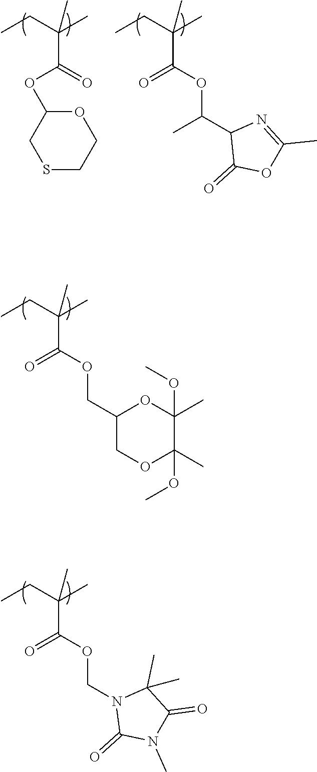

The resin (A) may have, in addition to the repeating units described above, various repeating units for the purpose of controlling dry etching resistance, suitability for a standard developer, adhesiveness to a substrate, a resist profile, resolution, heat resistance, sensitivity, etc.

Preferably, in the resin (A) (particularly when the composition is used as an actinic ray-sensitive or radiation-sensitive resin composition for ArF light), all the repeating units are composed of repeating units derived from compounds having an ethylenically unsaturated bond. In particular, it is also preferable that all the repeating units are composed of (meth)acrylate-based repeating units. In the resin (A) used in this case, all the repeating units may be methacrylate-based repeating units, or all the repeating units may be acrylate-based repeating units. Alternatively, the repeating units may each be a methacrylate-based repeating unit or an acrylate-based repeating unit. It is preferable that the content of the acrylate-based repeating units with respect to the total amount of the repeating units is 50% by mole or less.

The resin (A) can be synthesized by a routine method (for example, radical polymerization).

The weight average molecular weight of the resin (A) that is determined as a polystyrene-equivalent value by the GPC method is preferably 30,000 or less, more preferably 1,000 to 30,000, still more preferably 3,000 to 30,000, and particularly preferably 5,000 to 15,000.

The dispersity (molecular weight distribution) of the resin (A) is preferably 1 to 5, more preferably 1 to 3, still more preferably 1.2 to 3.0, and particularly preferably 1.2 to 2.0. The smaller the dispersity, the better the resolution and the resist profile, and the smoother the side surfaces of the resist pattern, so that better roughness quality is obtained.

In the resist composition, the content of the resin (A) with respect to the total mass of the solids in the resist composition is preferably 40.0 to 99.9% by mass and more preferably 60.0 to 90.0% by mass.

One type of resin (A) may be used alone, or a combination of a plurality of types may be used.

### [Acid Diffusion Control Agent]

The resist composition may include an acid diffusion control agent.

The acid diffusion control agent does not include the onium salt (II).

The acid diffusion control agent functions as a quencher that traps the acid generated from the photoacid generator etc. during exposure to light to thereby suppress the reaction of the acid decomposable resin with an excess portion of the generated acid in unexposed portions.

No particular limitation is imposed on the acid diffusion control agent, and examples thereof include a basic compound (CA), a low-molecular weight compound (CB) having a nitrogen atom and having a group that leaves by the action of an acid, and a compound (CC) whose acid diffusion control ability decreases or disappears when the compound (CC) is irradiated with actinic rays or radiation.

Examples of the compound (CC) include an onium salt compound (CD) that serves as a weak acid weaker than the photoacid generator and a basic compound (CE) whose basicity decreases or disappears upon irradiation with actinic rays or radiation.

Specific examples of the basic compound (CA) include those described in paragraphs [0132] to [0136] of WO2020/066824A, and specific examples of the basic compound (CE) whose basicity decreases or disappears upon irradiation with actinic rays or radiation include those described in paragraphs [0137] to [0155] of WO2020/066824A. Specific examples of the low-molecular weight compound (CB) having a nitrogen atom and having a group that leaves by the action of an acid include those described in paragraphs [0156] to [0163] of WO2020/066824A, and specific examples of an onium salt compound (CE) that has a nitrogen atom in its cationic moiety include those described in paragraph [0164] of WO2020/066824A.

Specific examples of the onium salt compound (CD) that serves as a weak acid weaker than the photoacid generator include those described in paragraphs [0305] to [0314] of WO2020/158337A.

In addition to the compounds described above, for example, known compounds disclosed in paragraphs [0627] to [0664] of US2016/0070167A, paragraphs [0095] to [0187] of US2015/0004544A, paragraphs [0403] to [0423] of US2016/0237190A, and paragraphs of US2016/0274458A can be preferably used as the acid diffusion control agent.

When the resist composition includes the acid diffusion control agent, the content of the acid diffusion control agent (the total content when a plurality of acid diffusion control agents are present) with respect to the total mass of the solids in the resist composition is preferably 0.1 to 20.0% by mass, more preferably 0.1 to 15.0% by mass, still more preferably 0.1 to 10.0% by mass, and particularly preferably 1.0 to 10.0% by mass.

One acid diffusion control agent may be used alone, or a combination of two or more acid diffusion control agents may be used.

### [Hydrophobic Resin]

The resist composition may include, in addition to the acid decomposable resin, a hydrophobic resin different from the acid decomposable resin.

Preferably, the hydrophobic resin is designed so as to segregate on the surface of a resist film. However, it is not always necessary that, unlike a surfactant, the hydrophobic resin have a hydrophilic group in its molecule and contribute to uniform mixing of polar and nonpolar substances.

The effects of the addition of the hydrophobic resin include, for example, control of the static and dynamic contact angles of water on the surface of the resist film and reduction of outgassing.

From the viewpoint of segregation of the hydrophobic resin in a surface layer of the film, the hydrophobic resin has preferably at least one of a fluorine atom, a silicon atom, or a CH₃ partial structure included in a side chain portion of the resin and has more preferably two or more of them. Preferably, the hydrophobic resin has a hydrocarbon group having 5 or more carbon atoms. Each of these groups may be present as a substituent in the main chain of the resin or its side chain.

Examples of the hydrophobic resin include compounds described in paragraphs [0275] to [0279] of WO2020/004306A.

When the resist composition includes the hydrophobic resin, its content with respect to the total mass of the solids in the resist composition is preferably 0.01 to 20% by mass, more preferably 0.1 to 15% by mass, still more preferably 0.1 to 10% by mass, and particularly preferably 0.1 to 8.0% by mass.

One hydrophobic resin may be used alone, or two or more hydrophobic resins may be used. When two or more hydrophobic resins are used in combination, it is preferable that the total content falls within the above preferred range.

### [Surfactant]

The resist composition may include a surfactant.

When the surfactant is included, the compound has better adhesiveness, and a pattern with less development defects can be formed.

The surfactant is preferably a fluorine-based surfactant and/or a silicon-based surfactant.

Examples of the fluorine-based surfactant and/or the silicon-based surfactant that can be used include surfactants disclosed in paragraphs [0218] and [0219] of WO2018/19395A.

When the resist composition includes the surfactant, its content with respect to the total mass of the solids in the resist composition is preferably 0.0001 to 2% by mass and more preferably 0.0005 to 1% by mass.

One surfactant may be used alone, or two or more surfactants may be used. When two or more surfactants are used in combination, it is preferable that the total content falls within the above preferred range.

### [Solvent]

The resist composition may include a solvent.

Preferably, the solvent includes at least one of (M₁) propylene glycol monoalkyl ether carboxylate or (M₂) at least one selected from the group consisting of propylene glycol monoalkyl ethers, lactates, acetates, alkoxypropionates, chain ketones, cyclic ketones, lactones, and alkylene carbonates. The solvent may further include a component other than the components (M₁) and (M₂).

The inventors have found that, when the above-described solvent and the above-described resin are used in combination, the coatability of the composition is improved, and a pattern with less development defects can be formed.

The reason for this is unclear, but the inventors consider that the reason may be as follows. The solubility of the resin in the above solvent, the boiling point of the solvent, and its viscosity are well-balanced, and therefore unevenness of the thickness of a film of the composition, the occurrence of precipitates during spin coating, etc. can be reduced.

The details of the components (M1) and (M2) are described in paragraphs [0218] to [0226] of WO2020/004306A.

When the solvent further includes a component other than the components (M1) and (M2), the content of the component other than the components (M1) and (M2) with respect to the total amount of the solvent is preferably 5 to 30% by mass.

The content of the solvent in the resist composition is determined such that the concentration of solid contents in the resist composition is preferably 30% by mass or less, more preferably 10% by mass or less, and still more preferably 2% by mass or less. The lower limit of the content is determined such that the concentration of solid contents is preferably 0.05% by mass or more, more preferably 0.1% by mass or more, and still more preferably 0.5% by mass or more. When the content is within the above range, the coatability of the resist composition can be further improved.

The content of the solvent with respect to the total mass of the resist composition is preferably 70 to 99.95% by mass, more preferably 90 to 99.9% by mass, and still more preferably 98 to 99.5% by mass.

One solvent may be used alone, or two or more solvents may be used. When two or more solvents are used, it is preferable that the total content falls within the above preferred range.

### [Additional Additives]

The resist composition may further include a dissolution inhibiting compound, a dye, a plasticizer, a photosensitizer, a light absorber, and/or a compound capable of increasing the solubility in a developer (such as an alicyclic or aliphatic compound including a carboxylic acid group).

The resist composition may further include a dissolution inhibiting compound. The “dissolution inhibiting compound” is a compound that has a molecular weight of 3000 or less and is decomposed by the action of an acid to cause the degree of solubility of the resist composition in an organic-based developer to decrease.

The resist composition is preferably used also as a photosensitive composition for EUV light.

The wavelength of the EUV light is 13.5 nm and is shorter than the wavelength of ArF light (wavelength: 193 nm) etc., and the number of incident photons when light exposure is performed at the same sensitivity is smaller. Therefore, the influence of “photon shot noise,” i.e., stochastic variations in the number of photons, is large, and this causes an increase in LWR and bridge defects. One method to reduce the photon shot noise is to increase the exposure value to increase the number of incident photons, but there is a trade-off with a demand for higher sensitivity.

When the value of A determined by general formula (1) is large, the efficiency of absorption of EUV light and electron beams by a resist film formed by the resist composition is high, and this is effective in reducing the photon shot noise. The value of A means the efficiency of absorption of EUV light and electron beams by mass ratio of the resist film.

A=([H]×0.04+[C]×1.0+[N]×2.1+[O]×3.6+[F]×5.6+[S]×1.5+[I]×39.5)/([H]×1+[C]×12+[N]×14+[O]×16+[F]×19+[S]×32+[I]×127) General formula (1):

The value of A is preferably 0.120 or more. As for the upper limit of the value of A, if the value of A is excessively large, the EUV light and electron beam transmittance of the resist film decreases, and the profile of an optical image in the resist film deteriorates, so that a good pattern shape is unlikely to be obtained. Therefore, the upper limit is preferably 0.240 or less and more preferably 0.220 or less.

In general formula (1), [H] represents the molar ratio of hydrogen atoms derived from the total solids in the actinic ray-sensitive or radiation-sensitive resin composition with respect to all the atoms in the total solids, and [C] represents the molar ratio of carbon atoms derived from the total solids in the actinic ray-sensitive or radiation-sensitive resin composition with respect to all the atoms in the total solids. [N] represents the molar ratio of nitrogen atoms derived from the total solids in the actinic ray-sensitive or radiation-sensitive resin composition with respect to all the atoms in the total solids, and [O] represents the molar ratio of oxygen atoms derived from the total solids in the actinic ray-sensitive or radiation-sensitive resin composition with respect to all the atoms in the total solids. [F] represents the molar ratio of fluorine atoms derived from the total solids in the actinic ray-sensitive or radiation-sensitive resin composition with respect to all the atoms in the total solids, and [S] represents the molar ratio of sulfur atoms derived from the total solids in the actinic ray-sensitive or radiation-sensitive resin composition with respect to all the atoms in the total solids. [I] represents the molar ratio of iodine atoms derived from the total solids in the actinic ray-sensitive or radiation-sensitive resin composition with respect to all the atoms in the total solids.

For example, when the resist composition includes the acid decomposable resin, onium salt (I) and onium salt (II), and the solvent, the acid decomposable resin and the onium salt (I) and onium salt (II) correspond to the solids. Specifically, all the atoms in the total solids correspond to the sum of all the atoms derived from the acid decomposable resin and all the atoms derived from the onium salt (I) and onium salt (II). For example, [H] represents the molar ratio of hydrogen atoms derived from the total solids with respect to all the atoms in the total solids. In the above example, [H] represents the total molar ratio of hydrogen atoms derived from the acid decomposable resin and hydrogen atoms derived from the compound (1) with respect to the sum of all the atoms derived from the acid decomposable resin and all the atoms derived from the compound (1).

When the structures of the constituent components of the total solids in the resist composition and their contents are known, the value of A can be computed by computing the ratio of the numbers of atoms included in the composition. Even when the constituent components are unknown, the ratio of the numbers of constituent atoms can be computed by subjecting a resist film obtained by evaporating the solvent component in the resist composition to an analytical method such as elemental analysis.

### <Resist Film Forming Method and Pattern Forming Method>

Preferably, the procedure of a pattern forming method using the resist composition includes the following steps.
Step 1: The step of forming a resist film on a substrate using the resist composition.
Step 2: The step of exposing the resist film to light.
Step 3: The step of developing the exposed resist film using a developer.

The procedure of each of the steps will next be described in detail.

### [Step 1: Resist Film Forming Step]

Step 1 is the step of forming a resist film on a substrate using the resist composition.

The definition of the resist composition is as described above.

Examples of the method for forming a resist film on a substrate using the resist composition include a method in which the resist composition is applied to the substrate.

Preferably, the resist composition is filtrated through a filter before the application as needed. The pore size of the filter is preferably 0.1 m or less, more preferably 0.05 m or less, and still more preferably 0.03 m or less. The filer is preferably a polytetrafluoroethylene-made filter, a polyethylene-made filter, or a nylon-made filter.

The resist composition can be applied to a substrate (e.g., a silicon substrate or a silicon dioxide coating) used for production of an integrated circuit element using an appropriate application method using a spinner, a coater, etc. The application method is preferably spin coating using a spinner. The number or revolutions when the spin coating using a spinner is performed is preferably 1000 to 3000 rpm.

After the application of the resist composition, the substrate may be dried to thereby form the resist film. If necessary, an undercoat film (an inorganic film, an organic film, or an antireflection film) may be formed as an underlayer of the resist film.

Examples of the drying method include a method in which the substrate is heated and dried. The heating may be performed using heating means included in at least one of an ordinary exposing device or an ordinary developing device or may be performed using a hot plate etc. The heating temperature is preferably 80 to 150° C., more preferably 80 to 140° C., and still more preferably 80 to 130° C. The heating time is preferably 30 to 1000 seconds, more preferably 60 to 800 seconds, and still more preferably 60 to 600 seconds.

The film thickness of the resist film is preferably 10 to 120 nm because a finer pattern can be formed with higher accuracy. In particular, when the resist film is exposed to EUV light, the film thickness of the resist film is more preferably 10 to 65 nm and still more preferably 15 to 50 nm.

A topcoat may be formed on the resist film using a topcoat composition.

It is preferable that the topcoat composition is immiscible with the resist film and can be uniformly applied to the upper surface of the resist film. No particular limitation is imposed on the topcoat, and a well-known topcoat can be formed using a well-known method. For example, the topcoat can be formed using a method described in paragraphs [0072] to [0082] of JP2014-059543A.

It is preferable, for example, that a topcoat including a basic compound described in JP2013-061648A is formed on the resist film. Specific examples of the basic compound that can be included in the topcoat include basic compounds that can be included in the resist composition.

It is also preferable that the topcoat includes a compound including at least one group or bond selected from the group consisting of an ether bond, a thioether bond, a hydroxy group, a thiol group, a carbonyl bond, and an ester bond.

### [Step 2: Exposure Step]

Step 2 is the step of exposing the resist film to light.

Examples of the light exposure method include a method in which the resist film formed is irradiated with actinic rays or radiation through a prescribed mask.

Examples of the actinic rays or radiation include infrared rays, visible rays, ultraviolet rays, far-ultraviolet rays, extreme ultraviolet rays, X rays, and electron beams.

The wavelength of the far-ultraviolet rays is preferably 250 nm or shorter, more preferably 220 nm or shorter, and still more preferably 1 to 200 nm. Specific examples of the actinic rays or radiation include KrF excimer laser light (248 nm), ArF excimer laser light (193 nm), F₂ excimer laser light (157 nm), EUV light (13 nm), X rays, and electron beams.

It is preferable to perform baking (heating) after the light exposure but before development. The baking facilitates the reaction in the exposed portions, and the sensitivity and the pattern shape are further improved.

The heating temperature is preferably 80 to 150° C., more preferably 80 to 140° C., and still more preferably 80 to 130° C.

The heating time is preferably 10 to 1000 seconds, more preferably 10 to 180 seconds, and still more preferably 30 to 120 seconds.

The heating may be performed using heating means included in at least one of an ordinary exposing device or an ordinary developing device or may be performed using a hot plate etc.

This step is referred to as post-exposure baking (PEB).

### [Step 3: Developing Step]

Step 3 is the step of developing the exposed resist film with a developer to form a pattern.

The developer may be an alkali developer or may be a developer including an organic solvent (hereinafter referred to as an “organic-based developer”).

Examples of the developing method include: a method in which the substrate is dipped into a bath filled with the developer for a prescribed time (a dipping method); a method in which the developer is placed on the surface of the substrate so as to bulge due to surface tension and left to stand for a prescribed time to develop the resist film (a puddle method); a method in which the developer is sprayed onto the surface of the substrate (a spraying method); and a method in which the developer is continuously discharged from a developer discharging nozzle onto the substrate rotating at a constant speed while the developer discharging nozzle is scanned at a constant speed (a dynamic dispensing method).

The step of replacing the solvent with another solvent to stop the development may be performed after the developing step.

No particular limitation is imposed on the developing time so long as the resin in unexposed portions is dissolved sufficiently, and the developing time is preferably 10 to 300 seconds and more preferably 20 to 120 seconds.

The temperature of the developer is preferably 0 to 50° C. and more preferably 15 to 35° C.

The alkali developer used is preferably an aqueous alkali solution including an alkali. Examples of the type of aqueous alkali solution include aqueous alkali solutions including quaternary ammonium salts typified by tetramethylammonium hydroxide, inorganic alkalis, primary amines, secondary amines, tertiary amines, alcohol amines, cyclic amines, etc. In particular, the alkali developer is preferably an aqueous solution of a quaternary ammonium salt typified by tetramethylammonium hydroxide (TMAH). An appropriate amount of an alcohol, a surfactant, etc. may be added to the alkali developer. The alkali concentration of the alkali developer is generally 0.1% to 20% by mass. The pH of the alkali developer is generally 10.0 to 15.0. The content of water in the alkali developer is preferably 51 to 99.95% by mass.

The organic-based developer is preferably a developer including at least one organic solvent selected from the group consisting of ketone-based solvents, ester-based solvents, alcohol-based solvents, amide-based solvents, ether-based solvents, and hydrocarbon-based solvents.

A mixture of a plurality of solvents selected from the above solvents may be used, or the organic-based developer may be mixed with water or a solvent other that the above solvents. The content of water in the developer with respect to the total mass of the developer is preferably less than 50% by mass, more preferably less than 20% by mass, and still more preferably less than 10% by mass, and it is particularly preferable that the developer includes substantially no water.

The content of the organic solvent in the organic-based developer with respect to the total mass of the developer is preferably 50 to 100% by mass, more preferably 80 to 100% by mass, still more preferably 90 to 100% by mass, and particularly preferably 95 to 100% by mass.

### [Additional Steps]

Preferably, the above pattern forming method further includes the step of, after step 3, washing with a rinsing solution.

Examples of the rinsing solution used in the rinsing step after the step of developing using the alkali developer include pure water. An appropriate amount of a surfactant may be added to the pure water.

An appropriate amount of a surfactant may be added to the rinsing solution.

No particular limitation is imposed on the rinsing solution used for the rinsing step after the step of developing using the alkali developer so long as the rinsing solution does not dissolve the pattern, and a solution including a general-purpose organic solvent can be used. Preferably, the rinsing solution used includes at least one organic solvent selected from the group consisting of hydrocarbon-based solvents, ketone-based solvents, ester-based solvents, alcohol-based solvents, amide-based solvents, and ether-based solvents.

No particular limitation is imposed on the method for the rinsing step, and examples thereof include: a method in which the rinsing solution is continuously discharged onto the substrate rotating at a constant speed (a spin coating method); a method in which the substrate is dipped into a bath filled with the rinsing solution for a prescribed time (a dipping method); and a method in which the rinsing solution is sprayed onto the surface of the substrate (a spraying method).

The pattern forming method of the invention may further include a heating (post-baking) step after the rinsing step. Through this step, the developer and the rinsing solution remaining between traces of the pattern and inside the pattern are removed by baking. Through this step, the resist pattern is annealed, and the effect of improving surface roughness of the pattern is obtained. The heating step after the rinsing step is performed at generally 40 to 250° C. (preferably 90 to 200° C.) for generally 10 seconds to 3 minutes (preferably 30 seconds to 2 minutes).

The pattern formed may be used as a mask to perform etching treatment on the substrate. Specifically, the pattern formed in step 3 may be used as a mask to process the substrate (or the underlayer film and the substrate) to thereby form a pattern on the substrate.

No particular limitation is imposed on the method for processing the substrate (or the underlayer film and the substrate). It is preferable that the pattern formed in step 3 is used as a mask and the substrate (or the underlayer film and the substrate) is dry-etched to form a pattern on the substrate. The dry etching is preferably oxygen plasma etching.

Preferably, the resist composition and various materials (such as the solvent, the developer, the rinsing solution, a composition for forming an antireflection film, and the composition for forming the topcoat) used in the pattern forming method of the invention include no impurities such as metals. The content of the impurities included in each of these materials with respect to the mass of the solids in the resist composition or the material is preferably 1 ppm by mass or less, more preferably 10 ppb by mass or less, still more preferably 100 ppt (parts per trillion) by mass or less, particularly preferably 10 ppt by mass or less, and most preferably 1 ppt by mass or less. Examples of the metal impurities include Na, K, Ca, Fe, Cu, Mg, Al, Li, Cr, Ni, Sn, Ag, As, Au, Ba, Cd, Co, Pb, Ti, V, W, and Zn.

Examples of a method for removing impurities such as metals from the above materials include filtration using a filter. The details of the filtration using a filer are described in paragraph [0321] of WO2020/004306A.

Examples of a method for reducing the amount of impurities such as metals included in the above materials include: a method in which raw materials including smaller amounts of metals are used as the raw materials forming the above materials; a method in which the raw materials forming the above materials are filtrated through a filter; and a method in which distillation is performed under the condition that contamination is reduced as much as possible, for example, by coating the inside of the device used with Teflon (registered trademark).

Besides the filtration using a filter, an adsorbent may be used to remove impurities. The filtration using a filter and the absorbent may be used in combination. The adsorbent used may be a well-known adsorbent, and examples of the adsorbent that can be used include inorganic-based adsorbents such as silica gel and zeolite and organic-based adsorbents such as activated carbon. To reduce the amount of impurities such as metals included in the above materials, it is necessary to prevent the metal impurities from mixing in the production process. Whether the metal impurities have been sufficiently removed from the production device can be checked by measuring the content of metal components included in a washing solution used to clean the production device. The content of the metal components included in the washing solution after use is preferably 100 ppt (parts per trillion) by mass or less, more preferably 10 ppt by mass or less, and still more preferably 1 ppt by mass or less.

An electrically conductive compound may be added to an organic treatment solution such as the rinsing solution in order to prevent failure of chemical solution pipes and various parts (such as filters, O-rings, and tubes) due to electrostatic charges and subsequent electrostatic discharge. Examples of the electrically conductive compound include methanol. From the viewpoint of maintaining preferred development characteristics or rinsing characteristics, the amount of the electrically conductive compound added is preferably 10% by mass or less and more preferably 5% by mass or less.

The chemical solution pipes used may be, for example, SUS (stainless steel) pipes or pipes coated with antistatic-treated polyethylene, antistatic-treated polypropylene, or an antistatic-treated fluorocarbon resin (such as polytetrafluoroethylene or a perfluoroalkoxy resin). Similarly, antistatic-treated polyethylene, antistatic-treated polypropylene, or an antistatic-treated fluorocarbon resin (such as polytetrafluoroethylene or a perfluoroalkoxy resin) may be used for the filters and the O-rings.

### <Method for Manufacturing Electronic Device>

The present invention also relates to a method for manufacturing an electronic device including the pattern forming method described above and to an electronic device manufactured by the manufacturing method.

The electronic device in the invention is preferably installed in electric and electronic devices (such as household electrical appliances, OA (Office Automation) devices, media-related devices, optical devices, and telecommunication devices).

### EXAMPLES

The present invention will be further described in detail by way of Examples.

Materials, amounts used, ratios, treatment details, treatment procedures, etc. shown in the following Examples can be appropriately changed so long as they do not depart from the gist of the invention. Therefore, the scope of the present invention should not be construed as limited to the following Examples.

### <Components of Resist Compositions>

Components included in resist compositions used in Examples and Comparative Examples are shown below.

### [Onium Salts (I)]

The structures of onium salts (I) ((I)-1 to (I)-18) used to prepare the resist compositions are shown below.

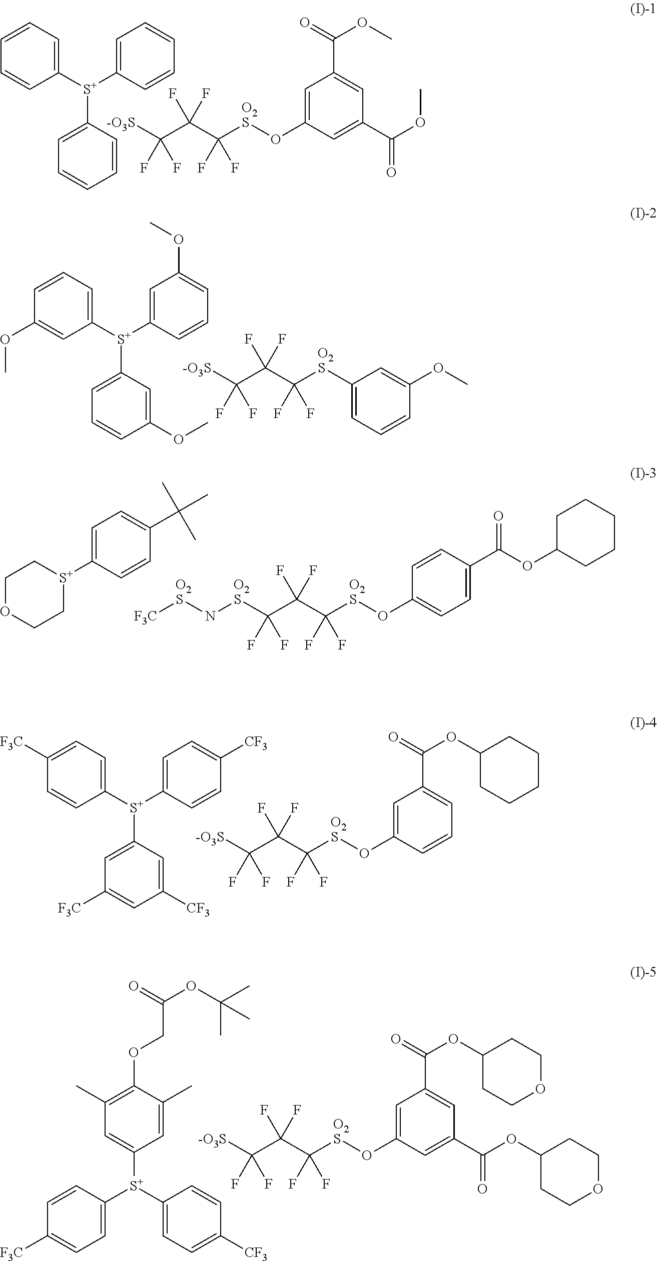

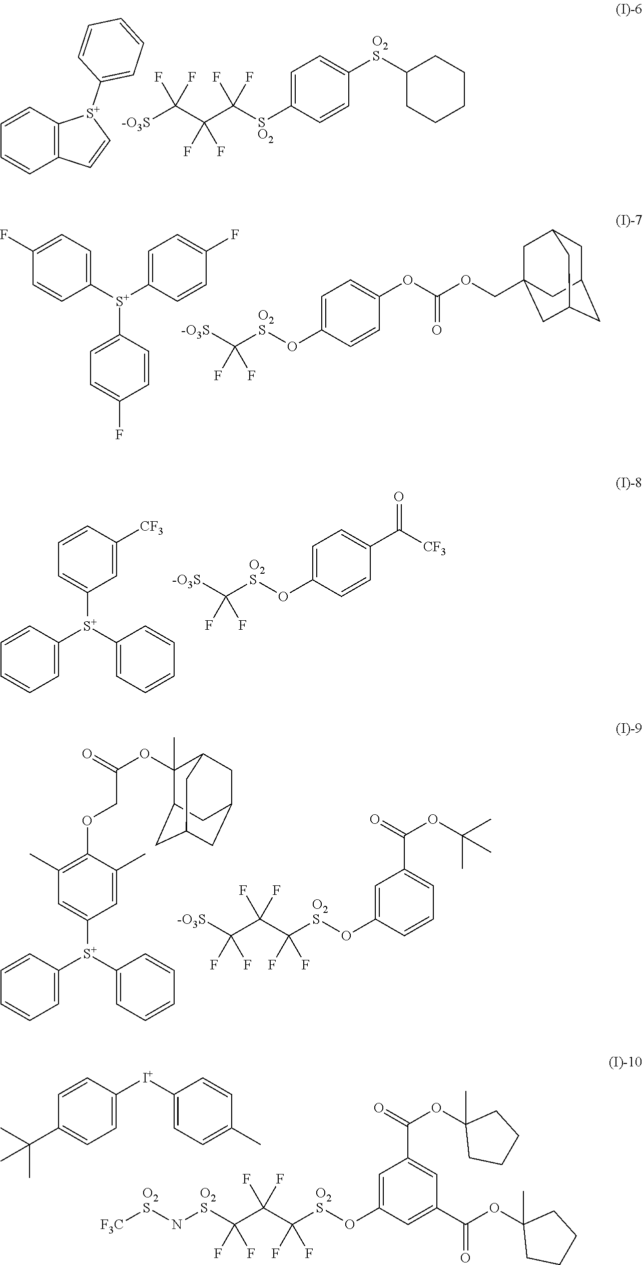

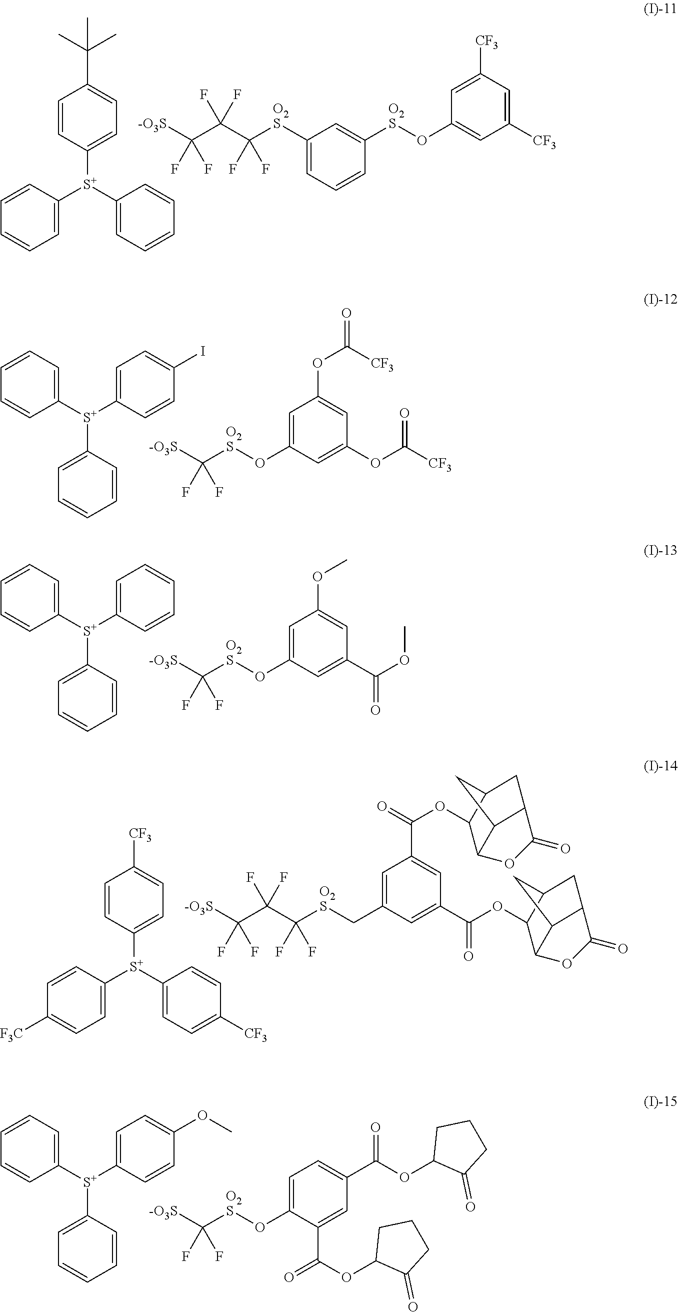

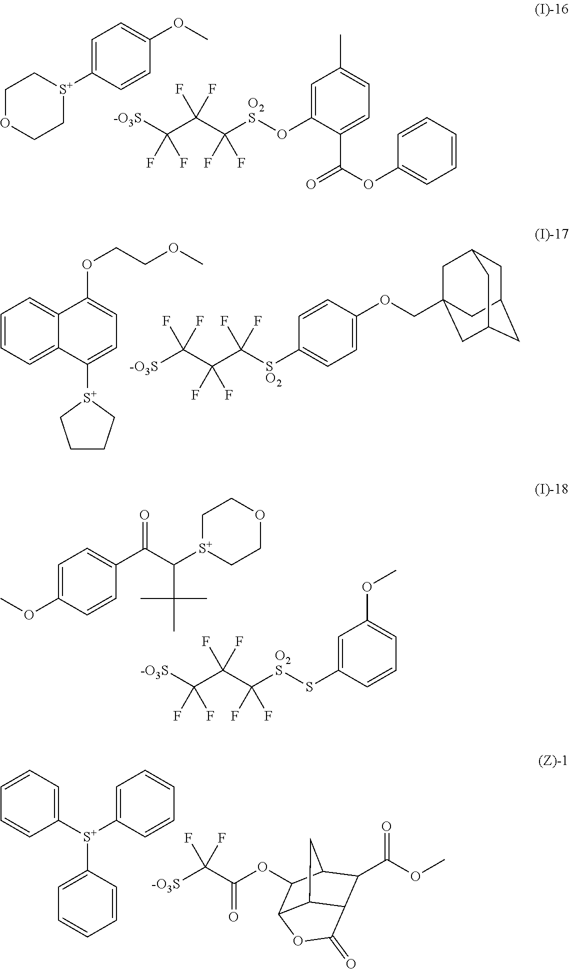

### (Synthesis of (I)-1)

Formula (I)-1-A below (614.8 g) was dissolved in tetrahydrofuran (600 mL), and the mixture was cooled to 0° C. to thereby obtain a precursor solution 1. 5-Hydroxyisophthalic acid (62.4 g) was dissolved in tetrahydrofuran (600 mL) to obtain a precursor solution 2. The cooled precursor solution 1 was maintained at 0° C., and the precursor solution 2 was added dropwise to the precursor solution 1. After completion of the dropwise addition, the reaction mixture was heated to room temperature and stirred for 2 hours to thereby obtain a reaction solution 1. Ethyl acetate (600 mL) was added to the reaction solution 1, and the organic phase was washed with 1N hydrochloric acid and water. Then the solvent was removed by evaporation to thereby obtain a crude product represented by formula (I)-1-B below. Next, the crude product (I)-1-B was dissolved in tetrahydrofuran (500 mL), and a 10% aqueous sodium hydrogencarbonate solution (500 mL) was added to the obtained solution. The mixture was stirred at 50° C. for 3 hours to thereby obtain a reaction solution 2. Ethyl acetate (1000 mL) was added to the reaction solution 2. Then the aqueous phase was removed from the reaction solution 2 using a separatory funnel, and ethyl acetate was further removed by evaporation to thereby obtain a crude product (I)-1-C. The crude product (I)-1-C was crystallized with diisopropyl ether to thereby obtain (I)-1-C(125 g) as a white solid (yield: 80%).

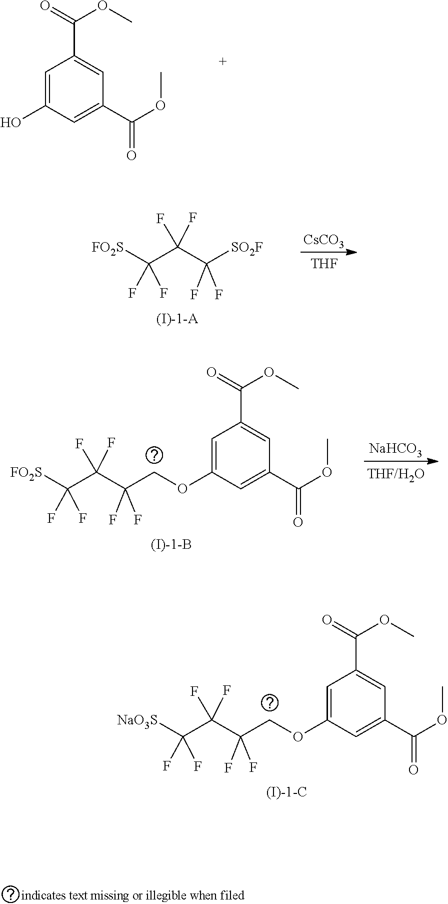

A 2L round bottom flask was charged with (I)-1-C(30.0 g), triphenylsulfonium bromide (19.6 g), dichloromethane (500 g), and ion exchanged water (500 g), and the mixture was stirred at room temperature for 30 minutes to thereby obtain a reaction solution 3. The aqueous phase was removed from the reaction solution 3 using a separatory funnel, and the organic phase was washed twice with ion exchanged water (350 mL). The solvent was removed from the washed reaction solution 3 by evaporation under reduced pressure, and the resulting product was crystallized with diisopropyl ether to thereby obtain an onium salt (I)-1 (41 g) as a white solid (yield: 94%).

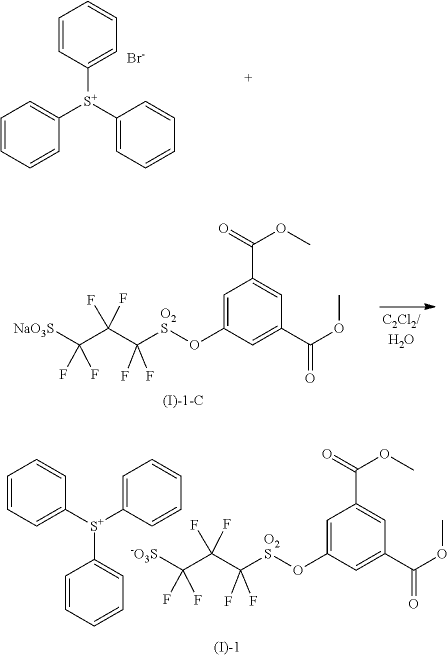

Onium salts (I)-2 to (I)-18 were synthesized using a method similar to the method for synthesizing the onium salt (I)-1.

### [Onium Salts (II)]

The structures of onium salts (II) ((II)-1 to (II)-18) used to prepare the resist compositions are shown below.

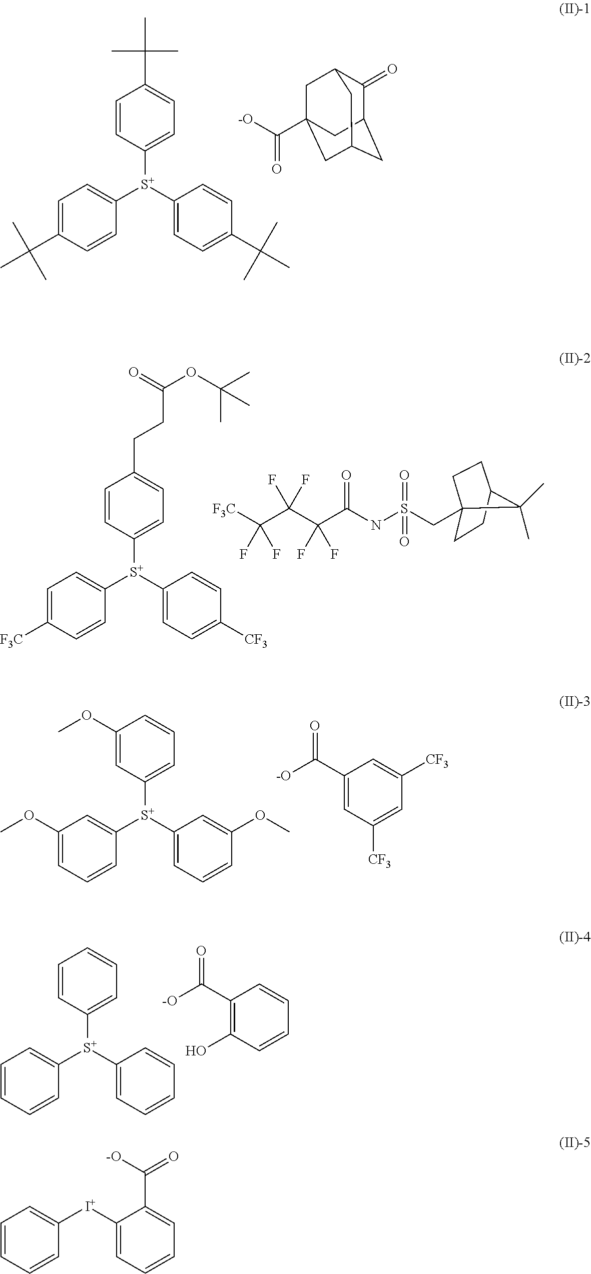

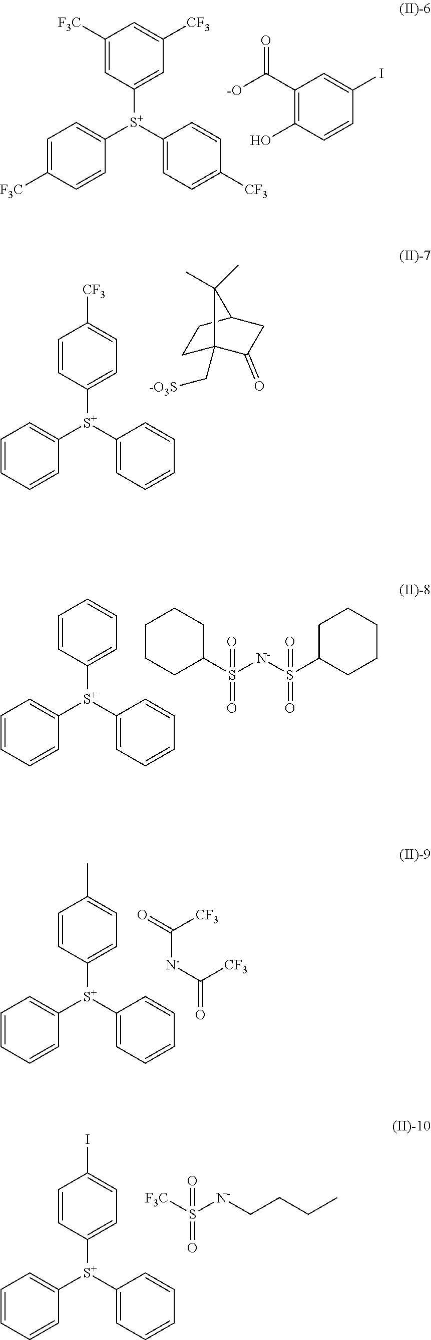

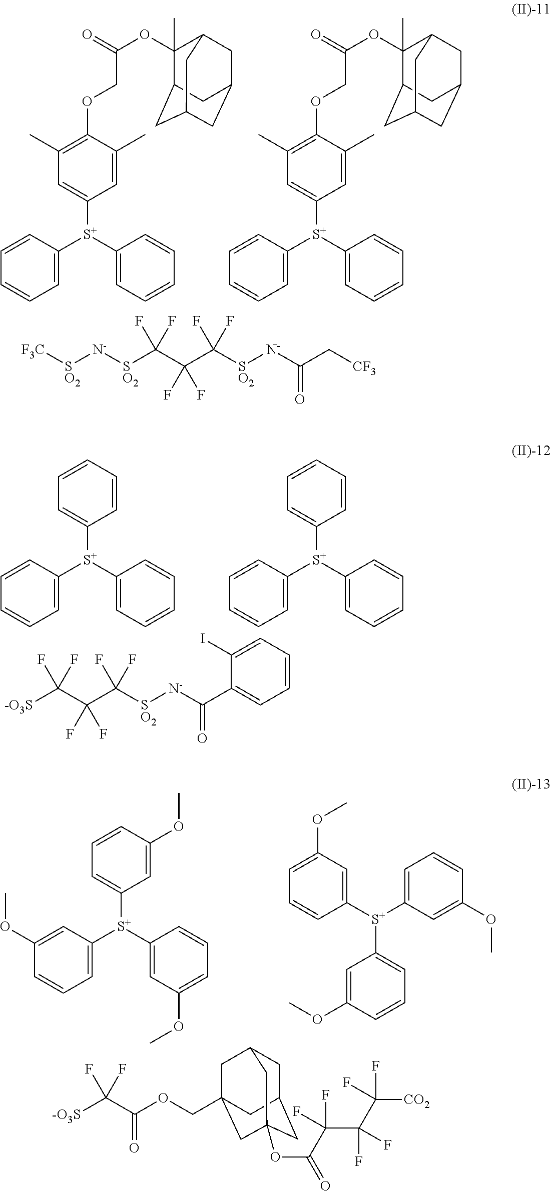

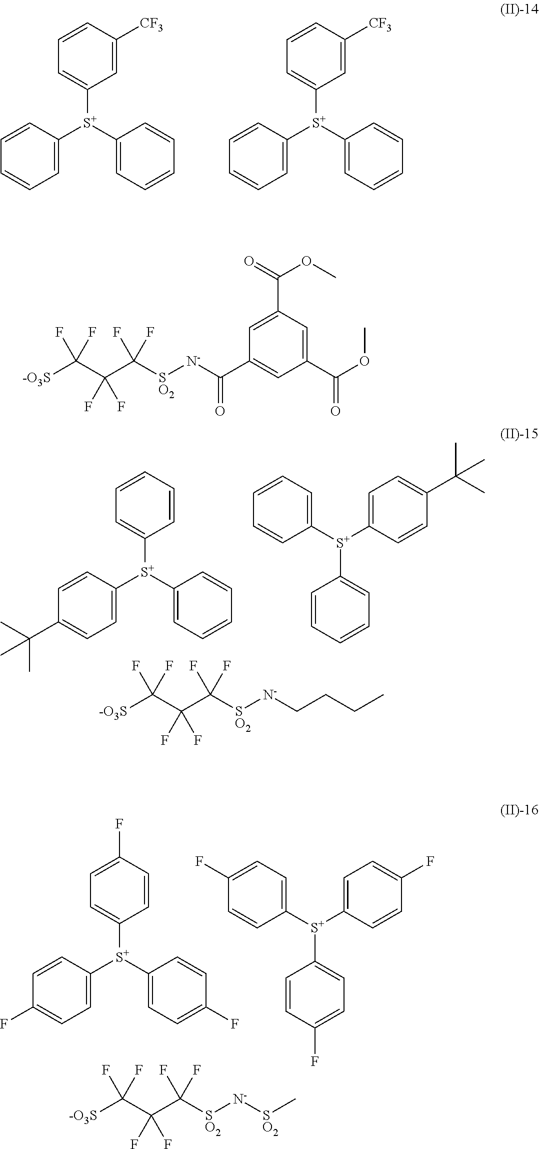

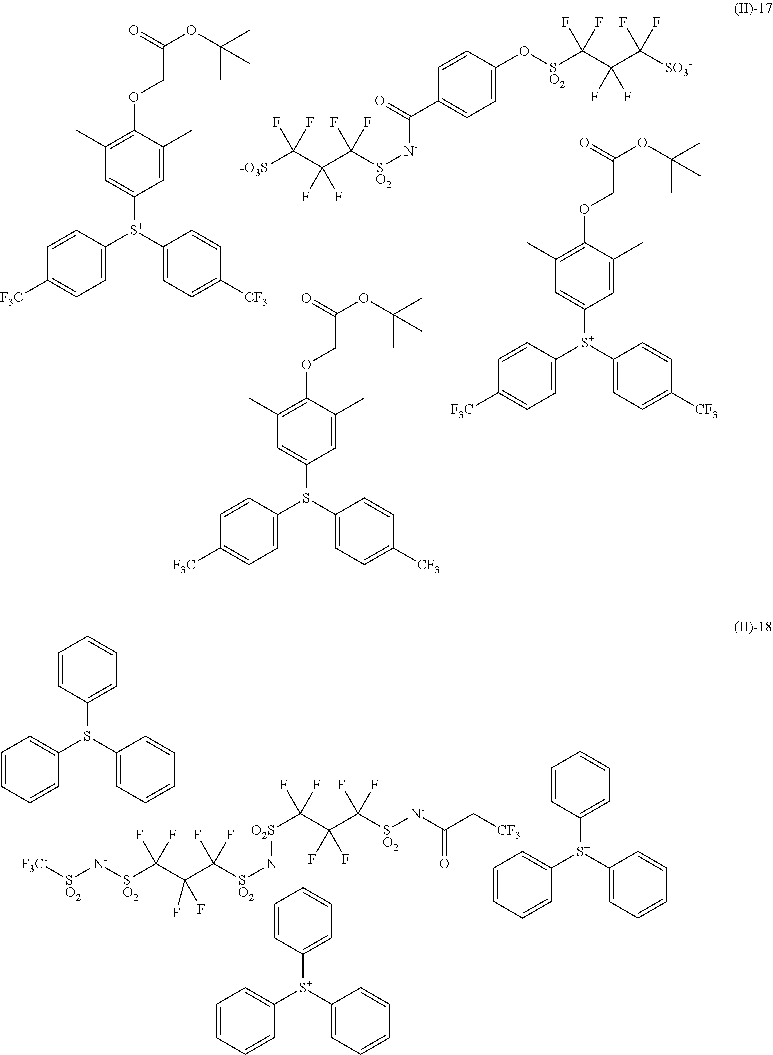

Onium salts (II)K to (II) to 18 were synthesized using a method similar to the method for synthesizing the onium salt (I)-1.

The acid dissociation constants (pKa) derived from acidic moieties represented by HA and obtained by replacing cationic moieties in the onium salts (I) and (II) with H⁺ are shown in Table 1.

In Table 1, the pKa values shown were determined using the software package 1 described above. When a plurality of acidic moieties are present in an onium salt (II), the pKa shown is the pKa of the acidic moiety derived from the structural moiety W.

TABLE 1
Table 1

| | |
|---|---|
| Onium salt (I) | Onium salt (II) |

| Type | pKa | Type | pKa |
|---|---|---|---|
| (I)-1 | −3.42 | (II)-1 | 3.02 |
| (I)-2 | −3.4 | (II)-2 | 0.15 |
| (I)-3 | −10.73 | (II)-3 | 3.34 |
| (I)-4 | −3.41 | (II)-4 | 3.01 |
| (I)-5 | −3.42 | (II)-5 | 3.09 |
| (I)-6 | 3.42 | (II)-6 | 3.08 |
| (I)-7 | −4.52 | (II)-7 | 1.17 |
| (I)-8 | −4.7 | (II)-8 | 0.25 |
| (I)-9 | −3.41 | (II)-9 | 3.04 |
| (I)-10 | −10.72 | (II)-10 | 7.51 |
| (I)-11 | −3.43 | (II)-11 | −0.37 |
| (I)-12 | −4.76 | (II)-12 | −0.44 |
| (I)-13 | −3.42 | (II)-13 | −0.37 |
| (I)-14 | −3.42 | (II)-14 | −0.63 |
| (I)-15 | −4.61 | (II)-15 | 8.12 |
| (I)-16 | −3.42 | (II)-16 | −3.13 |
| (I)-17 | −3.38 | (II)-17 | −0.9 |
| (I)-18 | −3.43 | (II)-18 | −0.37 |
| (Z)-1 | −3.35 | | |

### [Resins A]

Resins (A-1 to A-34 (corresponding to acid decomposable resins)) used to prepare the resist compositions are shown below. The resins A-1 to A-34 used were synthesized using a well-known method.

In Table 1, the “Molar ratio” columns each show the content (% by mole) of a repeating unit with respect to the total moles of repeating units.

In Table 1, the “Mw” column shows the weight average molecular weight.

In Table 1, the “Mw/Mn” column shows the dispersity.

The weight average molecular weight (Mw) and dispersity (Mw/Mn) (polystyrene equivalent values) of each of the resins A-1 to A-34 were measured by GPC (carrier: tetrahydrofuran (THF)). The compositional ratios (molar ratios) in each of the resins were measured by ¹³C-NMR (Nuclear Magnetic Resonance).

TABLE 2

| | Repeating | | Repeating | | Repeating | | Repeating | | | |
|---|---|---|---|---|---|---|---|---|---|---|
| | unit 1 | | unit 2 | | unit 3 | | unit 4 | | | |
| | | Molar | | Molar | | Molar | | Molar | | |
| | Type | ratio | Type | ratio | Type | ratio | Type | ratio | Mw | Mw/Mn |
| Resin A-1 | MB-10 | 50 | MA-16 | 50 | — | — | — | — | 8500 | 1.60 |
| Resin A-2 | MB-15 | 40 | MA-7 | 60 | — | — | — | — | 9000 | 1.70 |
| Resin A-3 | MB-7 | 30 | MB-14 | 10 | MA-6 | 60 | — | — | 7000 | 1.55 |
| Resin A-4 | MB-5 | 50 | MB-12 | 10 | MA-15 | 40 | — | — | 7500 | 1.55 |
| Resin A-5 | MB-3 | 20 | MB-20 | 40 | MA-4 | 40 | — | — | 7000 | 1.60 |
| Resin A-6 | MB-4 | 20 | MB-14 | 30 | MA-2 | 50 | — | — | 6500 | 1.63 |
| Resin A-7 | MB-6 | 30 | MB-19 | 10 | MA-3 | 60 | — | — | 9500 | 1.45 |
| Resin A-8 | MB-9 | 30 | MB-20 | 10 | MA-13 | 60 | — | — | 12000 | 1.65 |
| Resin A-9 | MB-4 | 20 | MB-19 | 20 | MA-2 | 60 | — | — | 6000 | 1.55 |
| Resin A-10 | MB-16 | 30 | MA-17 | 70 | — | — | — | — | 8000 | 1.40 |
| Resin A-11 | MB-4 | 30 | MB-31 | 30 | MA-4 | 40 | — | — | 6500 | 1.65 |
| Resin A-12 | MB-30 | 20 | MA-8 | 80 | — | — | — | — | 5500 | 1.65 |
| Resin A-13 | MB-13 | 50 | MA-10 | 50 | — | — | — | — | 15000 | 1.75 |
| Resin A-14 | MB-8 | 30 | MA-20 | 70 | — | — | — | — | 9000 | 1.60 |
| Resin A-15 | MB-18 | 30 | MB-29 | 30 | MA-14 | 40 | — | — | 8000 | 1.55 |
| Resin A-16 | MB-3 | 30 | MB-20 | 20 | MA-2 | 50 | — | — | 7500 | 1.70 |
| Resin A-17 | MB-1 | 30 | MB-26 | 30 | MA-11 | 40 | — | — | 18000 | 1.80 |
| Resin A-18 | MB-27 | 60 | MA-5 | 40 | — | — | — | — | 7500 | 1.65 |
| Resin A-19 | MB-17 | 30 | MB-24 | 10 | MA-19 | 60 | — | — | 8000 | 1.70 |
| Resin A-20 | MB-11 | 30 | MB-28 | 40 | MA-9 | 30 | — | — | 9500 | 1.80 |
| Resin A-21 | MB-3 | 20 | MB-20 | 10 | MB-32 | 10 | MA-2 | 60 | 10000 | 1.70 |
| Resin A-22 | MB-2 | 60 | MA-1 | 40 | — | — | — | — | 11000 | 1.65 |
| Resin A-23 | MB-21 | 50 | MA-12 | 50 | — | — | — | — | 6500 | 1.60 |
| Resin A-24 | MB-23 | 40 | MA-10 | 60 | — | — | — | — | 8000 | 1.55 |
| Resin A-25 | MB-1 | 20 | MB-19 | 20 | MA-2 | 60 | — | — | 8000 | 1.55 |
| Resin A-26 | MB-25 | 30 | MA-2 | 70 | — | — | — | — | 7500 | 1.60 |
| Resin A-27 | MB-3 | 40 | MA-3 | 60 | — | — | — | — | 9500 | 1.60 |
| Resin A-28 | MB-22 | 40 | MA-4 | 60 | — | — | — | — | 10000 | 1.70 |
| Resin A-29 | MB-3 | 30 | MB-33 | 20 | MA-2 | 50 | — | — | 6600 | 1.63 |
| Resin A-30 | MB-7 | 30 | MB-14 | 10 | MA-18 | 60 | — | — | 8100 | 1.65 |
| Resin A-31 | MB-1 | 20 | MB-19 | 20 | MA-21 | 60 | — | — | 6600 | 1.65 |
| Resin A-32 | MB-17 | 60 | MA-24 | 40 | — | — | — | — | 8200 | 1.71 |
| Resin A-33 | MB-4 | 20 | MB-19 | 20 | MA-23 | 60 | — | — | 6500 | 1.60 |
| Resin A-34 | MB-14 | 30 | MA-22 | 70 | — | — | — | — | 7200 | 1.58 |

The structures of monomers corresponding to the respective repeating units in the resins are shown below.

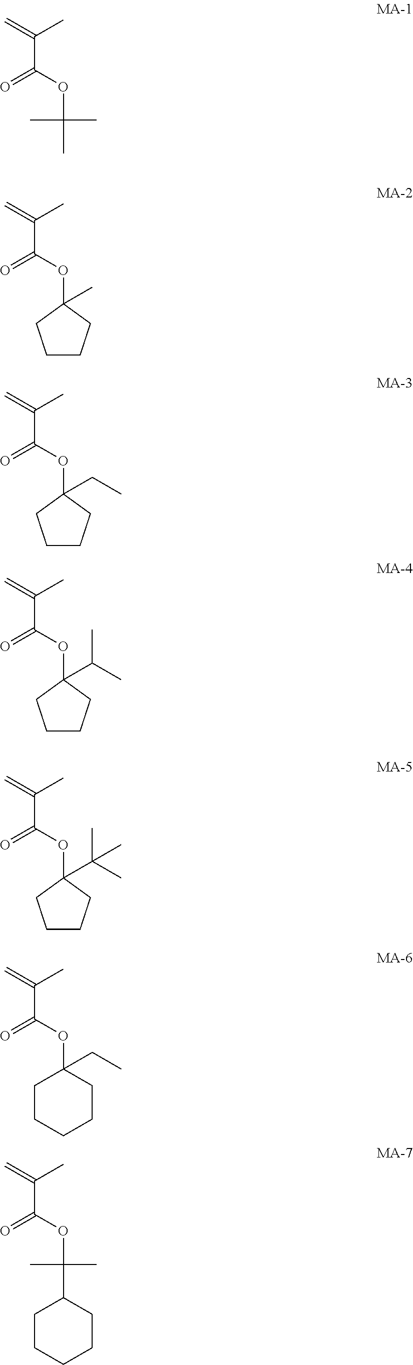

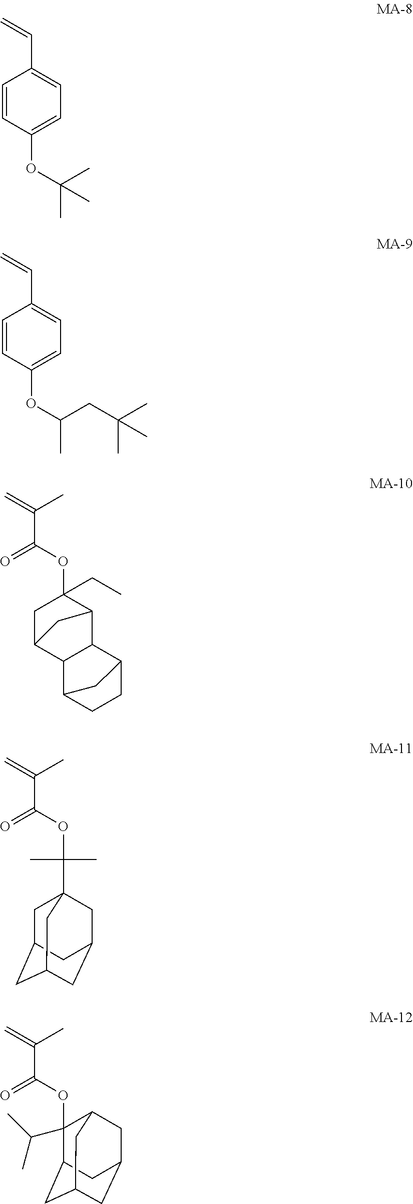

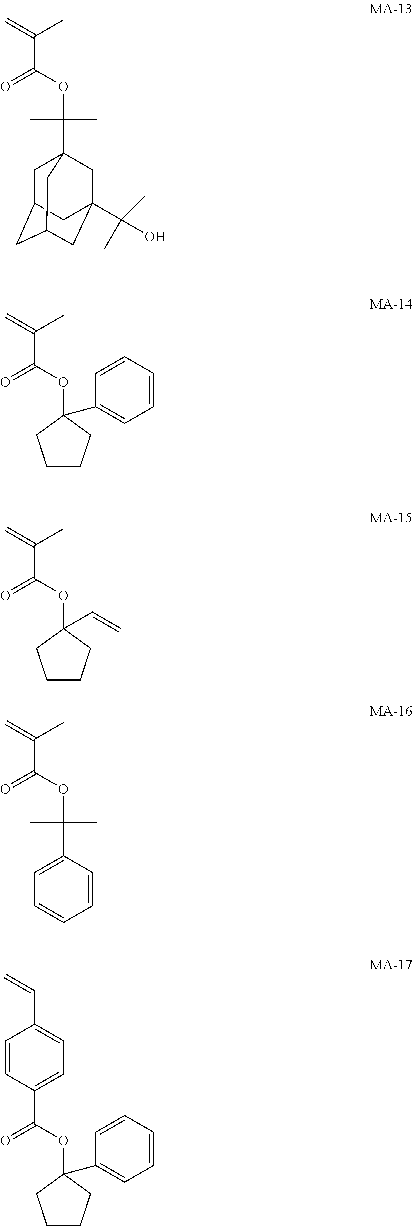

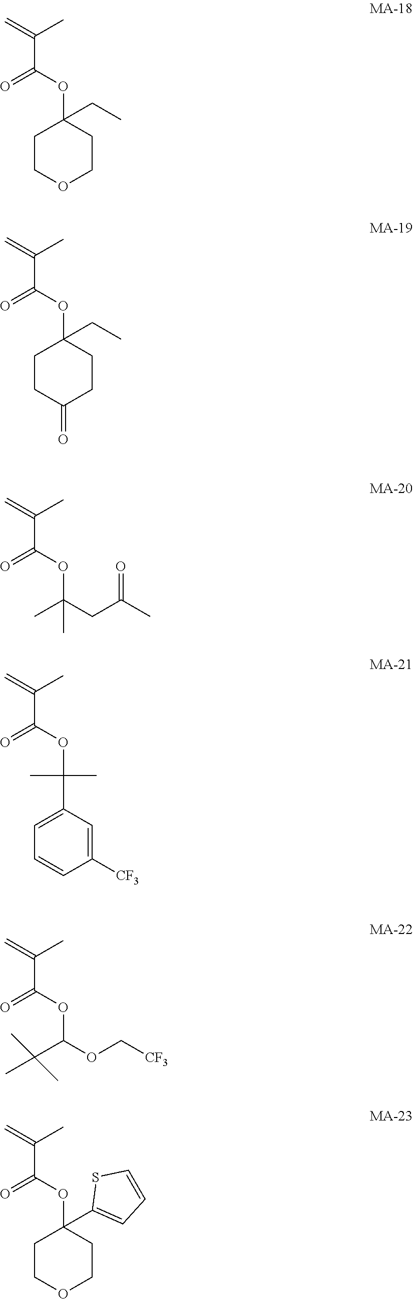

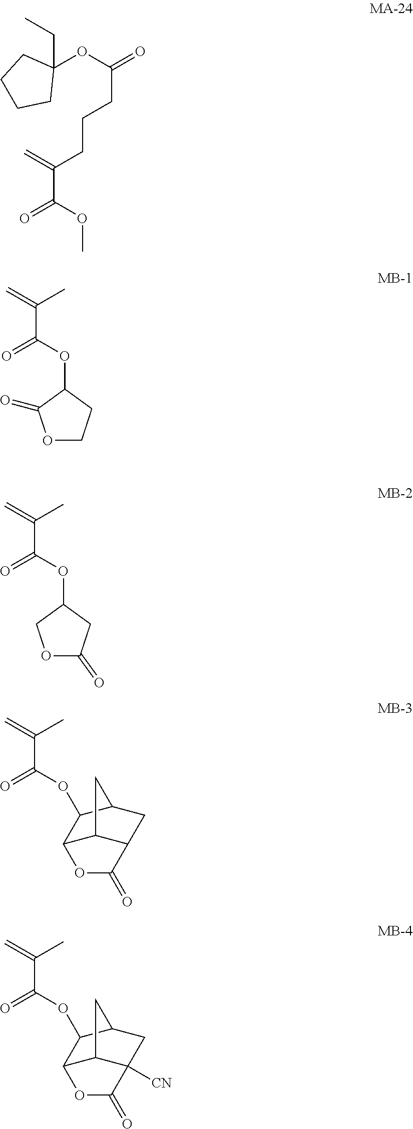

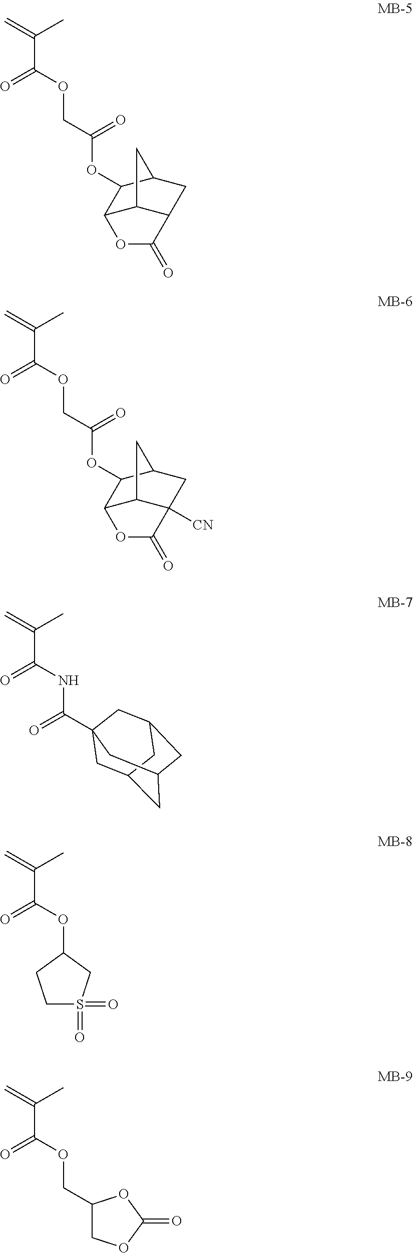

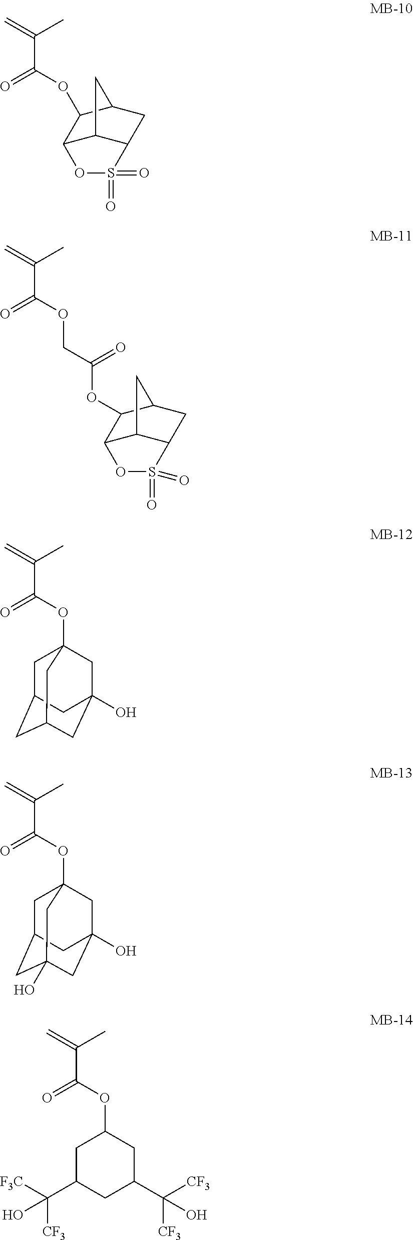

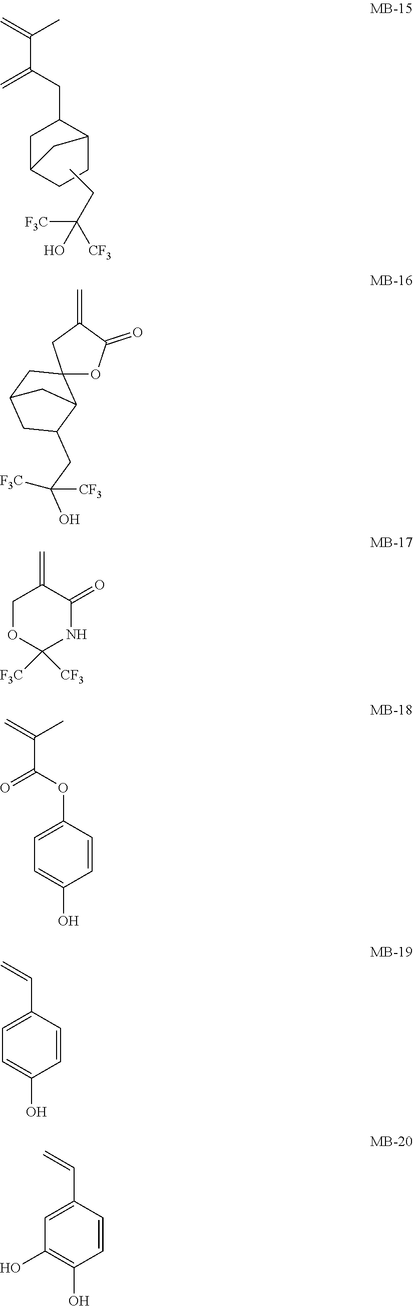

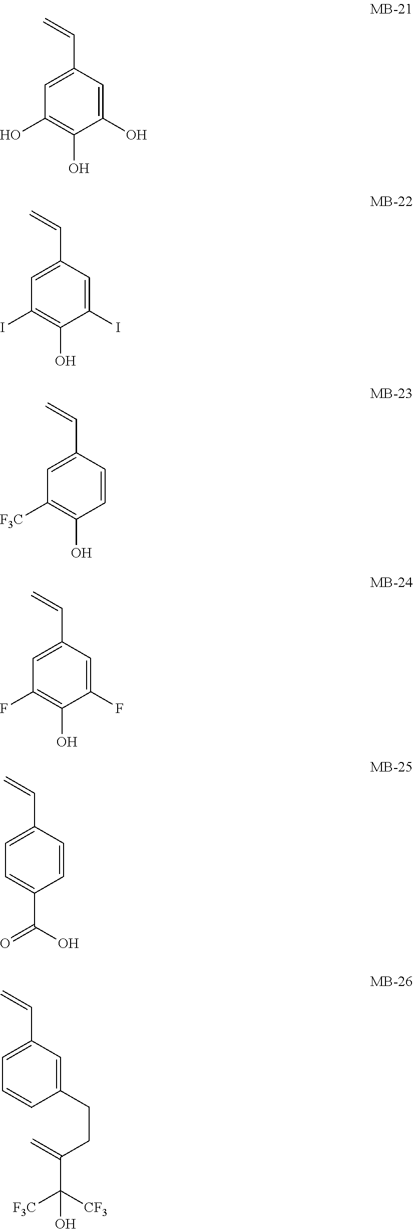

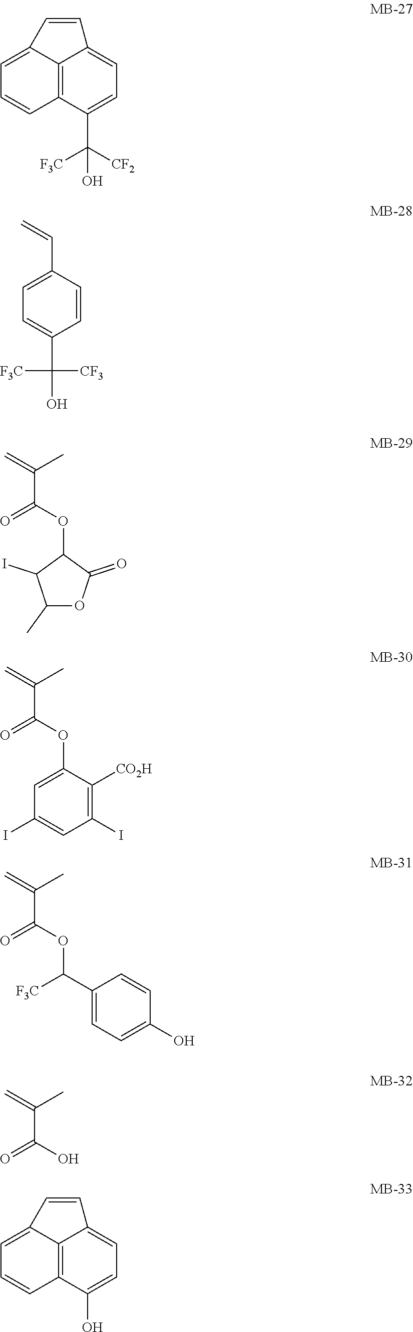

### [Acid Diffusion Control Agents B]

The structures of acid diffusion control agents (B-1 to B-4) used to prepare the resist compositions are shown below.

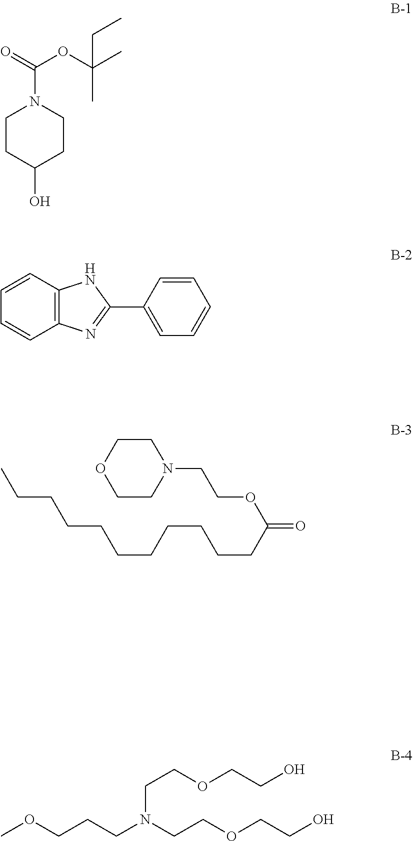 [SMILES: CCC(C)(C)OC(=O)N1CCC(O)CC1 ; c1ccc(-c2nc3ccccc3[nH]2)cc1 ; CCCCCCCCCCCC(=O)OCCN1CCOCC1 ; COCCCN(CCOCCO)CCOCCO]

### [Hydrophobic Resins C]

Hydrophobic resins C (C-1 to C-8) used to prepare the resist compositions are shown below.

In Table 3, the “Molar ratio” columns each show the content (% by mole) of a repeating unit with respect to the total moles of repeating units.

In Table 3, the “Mw” column shows the weight average molecular weight.

In Table 3, the “Mw/Mn” column shows the dispersity.

The weight average molecular weight (Mw) and dispersity (Mw/Mn) (polystyrene equivalent values) of each of the resins C-1 to C-8 were measured by GPC (carrier: tetrahydrofuran (THF)). The compositional ratios (molar ratios) in each of the resins were measured by ¹³C-NMR (Nuclear Magnetic Resonance).

TABLE 3

| | Repeating unit | | Repeating unit | | Repeating unit | | Repeating unit | | | |
|---|---|---|---|---|---|---|---|---|---|---|
| | 1 | | 2 | | 3 | | 4 | | | |
| | | Molar | | Molar | | Molar | | Molar | | |
| Table 3 | Type | ratio | Type | ratio | Type | ratio | Type | ratio | Mw | Mw/Mn |
| Resin C-1 | MC-9 | 50 | MC-1 | 50 | — | — | — | — | 12000 | 1.5 |
| Resin C-2 | MC-2 | 40 | MC-8 | 50 | MC-5 | 5 | MC-11 | 5 | 6000 | 1.3 |
| Resin C-3 | MC-6 | 50 | MC-2 | 50 | — | — | — | — | 15000 | 1.5 |
| Resin C-4 | MC-3 | 100 | — | — | — | — | — | — | 23000 | 1.7 |
| Resin C-5 | MC-8 | 10 | MC-10 | 85 | MC-5 | 5 | — | — | 11000 | 1.4 |
| Resin C-6 | MC-4 | 80 | MC-7 | 20 | — | — | — | — | 13000 | 1.4 |
| Resin C-7 | MC-3 | 50 | MC-12 | 50 | — | — | — | — | 12000 | 1.5 |
| Resin C-8 | MC-2 | 50 | MC-13 | 50 | — | — | — | — | 10000 | 1.6 |

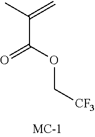

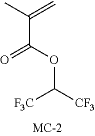

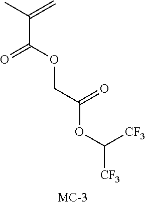

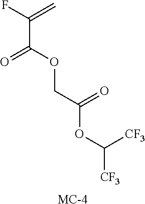

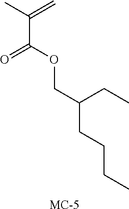

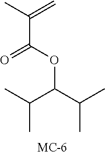

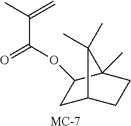

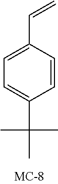

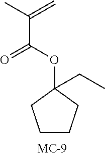

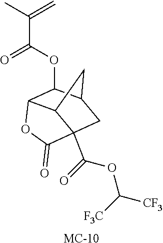

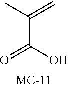

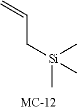

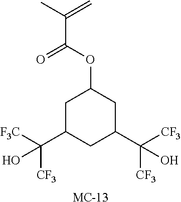

### [Surfactants E]

Surfactants E (E-1 to E-3) used to prepare the resist compositions are shown below.
E-1: MEGAFACE F176 (manufactured by DIC Corporation, fluorine-based surfactant)
E-2: MEGAFACE R₀₈ (manufactured by DIC Corporation, fluorine and silicon-based surfactant)
E-3: PF656 (manufactured by OMNOVA, fluorine-based surfactant)

### [Solvents F]

Solvents F (F-1 to F-9) used to prepare the resist compositions are shown below.
F-1: Propylene glycol monomethyl ether acetate (PGMEA)
F-2: Propylene glycol monomethyl ether (PGME)
F-3: Propylene glycol monoethyl ether (PGEE)
F-4: Cyclohexanone
F-5: Cyclopentanone
F-6: 2-Heptanone
F-7: Ethyl lactate
F-8: 7-Butyrolactone
F-9: Propylene carbonate

### <Preparation and Application of Resist Compositions>

Components shown in Table 4 below were mixed such that the concentration of solids was 2% by mass. The solution mixture obtained was filtered by causing the mixture to pass through a polyethylene-made filter with a pore size of 0.02 m to thereby prepare a resist composition.

The “solids” mean components other than the solvent.

One of these resist compositions was applied to a 6-inch Si (silicon) wafer treated in advance with hexamethyldisilazane (HMDS) using a spin coater “Mark 8” manufactured by Tokyo Electron Ltd. and dried on a hot plate at 130° C. for 300 seconds to thereby obtain a resist film having a film thickness of 30 nm.

Here, 1 inch is 0.0254 m.

In Table 4, entries separated by “/” in the type columns mean that a plurality of compounds are included as the corresponding material, and entries separated by “/” in the % by mass columns indicate the contents of the respective compounds. For example, the “Onium salt (I)” in “Re-26” includes “(I)-1” and “(I)-14,” and their contents are “13.0” and “13.0”% by mass, respectively.

In Table 4, the “% by mass” columns each show the content (% by mass) of a solid component with respect to the total mass of solids. The solids are components other than the solvent.

In Table 4, the “Mixing ratio” column in the “Solvent” column shows the mixing ratio (mass ratio) of solvents.

TABLE 4

| | | | | | | | Acid diffusion | | Hydrophobic | | | | | |
|---|---|---|---|---|---|---|---|---|---|---|---|---|---|---|
| Resist | Onium salt (I) | | Onium salt (II) | | Resin A | | control agent B | | resin C | | Surfactant E | | Solvent F | |
| composition | Type | % by mass | Type | % by mass | Type | % by mass | Type | % by mass | Type | % by mass | Type | % by mass | Type | Mixing ratio |
| Re-1 | (I)-1 | 26.0 | (II)-1 | 10.5 | A-25 | 63.5 | — | — | — | — | — | — | F-1/F-6 | 40/60 |
| Re-2 | (I)-1 | 22.0 | (II)-2 | 14.8 | A-7 | 63.2 | — | — | — | — | — | — | F-4 | 100 |
| Re-3 | (I)-1 | 24.5 | (II)-8 | 9.0 | A-34 | 66.5 | — | — | — | — | — | — | F-1 | 100 |
| Re-4 | (I)-1 | 12.0 | (II)-12 | 17.3 | A-3 | 69.6 | — | — | C-8 | 1.1 | — | — | F-1 | 100 |
| Re-5 | (I)-2 | 28.0 | (II)-5 | 6.2 | A-6 | 63.9 | — | — | C-5 | 1.9 | — | — | F-1/F-3 | 80/20 |
| Re-6 | (I)-3 | 30.0 | (II)-3 | 10.5 | A-20 | 57.1 | — | — | C-4 | 2.4 | — | — | F-1/F-9 | 90/10 |
| Re-7 | (I)-4 | 16.3 | (II)-14 | 19.2 | A-12 | 64.5 | — | — | — | — | — | — | F-1/F-7 | 80/20 |
| Re-8 | (I)-5 | 6.0 | (II)-17 | 40.0 | A-9 | 53.8 | — | — | — | — | E-1/E-2 | 0.1/0.1 | F-1/F-8 | 85/15 |
| Re-9 | (I)-6 | 4.0 | (II)-18 | 17.5 | A-15 | 78.5 | — | — | — | — | — | — | F-4 | 100 |
| Re-10 | (I)-7 | 40.0 | (II)-9 | 12.5 | A-30 | 46 | — | — | C-3 | 1.5 | — | — | F-1/F-5 | 50/50 |
| Re-11 | (I)-8 | 36.5 | (II)-4 | 10.1 | A-33 | 53.4 | — | — | — | — | — | — | F-1 | 100 |
| Re-12 | (I)-9 | 26.5 | (II)-6 | 9.0 | A-12 | 64.5 | — | — | — | — | — | — | F-1/F-2/F-8 | 70/25/5 |
| Re-13 | (I)-10 | 10.0 | (II)-11 | 13.4 | A-1 | 76.6 | — | — | — | — | — | — | F-1/F-6 | 40/60 |
| Re-14 | (I)-11 | 31.5 | (II)-7 | 8.5 | A-2 | 58 | — | — | C-1 | 2.0 | — | — | F-1/F-5 | 50/50 |
| Re-15 | (I)-12 | 9.5 | (II)-13 | 10.5 | A-27 | 80 | — | — | — | — | — | — | F-1 | 100 |
| Re-16 | (I)-13 | 23.0 | (II)-6 | 10.8 | A-31 | 66.2 | — | — | — | — | — | — | F-1/F-2/F-8 | 70/25/5 |
| Re-17 | (I)-14 | 13.0 | (II)-10 | 3.0 | A-5 | 83.9 | — | — | — | — | E-1 | 0.1 | F-7 | 100 |
| Re-18 | (I)-15 | 16.0 | (II)-15 | 20.0 | A-23 | 64 | — | — | — | — | — | — | F-1/F-8 | 85/15 |
| Re-19 | (I)-16 | 8.5 | (II)-1 | 3.2 | A-29 | 88.3 | — | — | — | — | — | — | F-1/F-2 | 70/30 |
| Re-20 | (I)-17 | 22.5 | (II)-2 | 13.4 | A-17 | 64 | — | — | — | — | E-2 | 0.1 | F-1/F-5 | 50/50 |
| Re-21 | (I)-18 | 25.0 | (II)-7 | 8.5 | A-10 | 63.1 | — | — | C-6 | 3.4 | — | — | F-1/F-2 | 70/30 |
| Re-22 | (I)-14 | 31.0 | (II)-16 | 20.4 | A-4 | 48.6 | — | — | — | | — | — | F-1/F-8 | 85/15 |
| Re-23 | (I)-5 | 29.0 | (II)-1 | 6.8 | A-32 | 61.3 | — | — | C-7 | 2.9 | — | — | F-1 | 100 |
| Re-24 | (I)-4 | 27.5 | (II)-1 | 7.9 | A-19 | 63.5 | — | — | C-2 | 1.1 | — | — | F-4 | 100 |
| Re-25 | (I)-2 | 31.0 | (II)-1 | 13.0 | A-18 | 56 | — | — | — | — | — | — | F-1/F-6 | 40/60 |
| Re-26 | (I)-1/(I)-14 | 13.0/13.0 | (II)-1 | 10.5 | A-8 | 63.5 | — | — | — | — | — | — | F-4 | 100 |
| Re-27 | (I)-10 | 20.0 | (II)-2/(II)-14 | 4.0/12.5 | A-14 | 63.5 | — | — | — | — | — | — | F-4 | 100 |
| Re-28 | (I)-8 | 23.5 | (II)-6 | 12.6 | A-22/A-24 | 30.0/33.9 | — | — | — | — | — | — | F-1 | 100 |
| Re-29 | (I)-7 | 26.0 | (II)-4 | 6.4 | A-11 | 66 | B-2 | 1.6 | — | — | — | — | F-1/F-8 | 85/15 |
| Re-30 | (I)-6 | 31.0 | (II)-5 | 5.2 | A-16 | 62.7 | B-4 | 1.1 | — | — | — | — | F-7 | 100 |
| Re-31 | (I)-18 | 18.5 | (II)-8 | 6.0 | A-26 | 74.7 | B-3 | 0.8 | — | — | — | — | F-1/F-8 | 85/15 |
| Re-32 | (I)-12 | 29.0 | (II)-2 | 11.0 | A-13 | 58.8 | B-1 | 1.2 | — | — | — | — | F-1/F-2 | 70/30 |
| Re-33 | (I)-5 | 13.0 | (II)-2/(II)-17 | 3.2/28.6 | A-21 | 55.2 | — | — | — | — | — | — | F-1/F-3 | 90/10 |
| Re-34 | (I)-13 | 32.0 | (II)-7 | 6.0 | A-28 | 62 | — | — | — | — | — | — | F-4 | 100 |
| Re-35 | (I)-1 | 6.8 | (II)-11/(II)-18 | 15.7/15.4 | A-1 | 62.1 | — | — | — | — | — | — | F-1/F-8 | 85/15 |
| Re-C1 | (Z)-1 | 28.0 | (II)-1 | 13.2 | A-25 | 58.8 | — | — | — | — | — | — | F-1/F-6 | 40/60 |
| Re-C2 | (I)-1 | 26.0 | — | — | A-25 | 70.5 | B-1 | 3.5 | — | — | — | — | F-1/F-6 | 40/60 |

### <Exposure and Development>

### [Euv Exposure]

A wafter coated with one of the resin films obtained above was subjected to pattern exposure using an EUV exposure device (Micro Exposure Tool, NA (numerical aperture): 0.3, Quadrupole, outer sigma: 0.68, inner sigma: 0.36) manufactured by Exitech. The exposure mask used was a mask with a line width of 20 nm and a 1:1 line and space pattern.

### [Organic Solvent Development]

The wafer exposed to light was heated on a hot plate at 90° C. for 60 seconds, developed with n-butyl acetate for 30 seconds, and spin-dried to thereby obtain a negative-type resist pattern.

Through the above procedure, resist patterns in Examples 1-1 to 1-35 and Comparative Examples 1-1 and 1-2 shown in Table 5 described later were obtained.

### [Alkali Development]

A wafer exposed to light was heated on a hot plate at 100° C. for 90 second, immersed in a 2.38% by mass aqueous tetramethylammonium hydroxide (TMAH) solution for 60 seconds, and rinsed with water for 30 seconds. Then the wafer was rotated at a rotation speed of 4000 rpm for 30 seconds and baked at 95° C. for 60 seconds to dry the water, and a positive-type wafer was thereby obtained.

Through the above procedure, resist patterns in Examples 2-1 to 2-35 and Comparative Examples 2-1 and 2-2 shown in Table 6 described later were obtained.

### <Evaluation of Cross-Sectional Rectangularity (1)>

The resist patterns in Examples and Comparative Examples (Examples 1-1 to 1-35 and Comparative Examples 1-1 and 1-2) obtained by the organic solvent development were evaluated according to the following procedure.

For each of the Examples and Comparative Examples, a cross-sectional shape of the obtained line pattern having an average line width of 20 nm was observed under a critical dimension scanning electron microscope (SEM, S-9380II manufactured by Hitachi, Ltd.), and the pattern line width Lb in a bottom portion of the resist pattern and the pattern line width La in an upper portion of the resist pattern were measured. The cross-sectional rectangularity of the pattern shape was evaluated based on the value of La/Lb according to the following criteria. S is the best, and E is the worst. A rating of D or higher is practically preferable.

### [Evaluation Criteria]

S: 1.00<(La/Lb)<1.01
A: 1.01<(La/Lb)<1.02
B: 1.02<(La/Lb)<1.03
C: 1.03<(La/Lb)<1.04
D: 1.04<(La/Lb)<1.05
E: 1.05<(La/Lb)

### <Evaluation of Cross-Sectional Rectangularity (2)>

The resist patterns in Examples and Comparative Examples (Examples 2-1 to 2-35 and Comparative Examples 2-1 and 2-2) obtained by the alkali development were evaluated according to the following procedure.

For each of the Examples and Comparative Examples, a cross-sectional shape of the obtained line pattern having an average line width of 20 nm was observed under a critical dimension scanning electron microscope (SEM, S-9380II manufactured by Hitachi, Ltd.), and the pattern line width L_{b} in a bottom portion of the resist pattern and the pattern line width La in an upper portion of the resist pattern were measured. The cross-sectional rectangularity of the pattern shape was evaluated based on the value of La/L_{b} according to the following criteria. S is the best, and E is the worst. A rating of D or higher is practically preferable.

### [Evaluation Criteria]

S: 1.00<(Lb/La)<1.01
A: 1.01<(Lb/La)<1.02
B: 1.02<(Lb/La)<1.03
C: 1.03<(Lb/La)<1.04
D: 1.04<(Lb/La)<1.05
E: 1.05<(Lb/La)

Table 5 shows the results of the evaluation of the cross-sectional rectangularity in Examples 1-1 to 1 to 35 and Comparative Examples 1-1 and 1-2 (alkali development). Table 6 shows the results of the evaluation of the cross-sectional rectangularity in Examples 2-1 to 2-35 and Comparative Examples 2-1 and 2-2 (organic solvent development).

In Tables 5 and 6, “A” to “D” in the “Condition 1” column each indicate that the onium salt (I) used for the resist composition is as follows.
A: The onium salt (I) generates the acid represented by formula (1) in which n is 2 to 5 and R_{S} in formula (1) represents —CO—O—R_{S1} or —O—CO—R_{S1}.
B: The onium salt (I) generates the acid represented by formula (1) in which n is 1 and R_{S} in formula (1) represents —CO—R_{S1}, —CO—O—R_{S1}, —O—CO—R_{S1}, —O—CO—O—R_{S1}, —SO₂—R_{S1}, or —SO₃—R_{S1} or in which n is 2 to 5 and only one of R_{S}'s in formula (1) represents —CO—O—R_{S1} or —O—CO—R_{S1}.
C: The onium salt (I) generates the acid represented by formula (1) in which R_{S} in formula (1) represents —O—R_{S1}.
D: The onium salt (I) does not generate the acid represented by formula (1).

In Tables 5 and 6, “^{A}” to “C” in the “Condition 2” column each indicate that the onium salt (II) used for the resist composition is as follows.
A: The anionic moiety A (the anionic moiety having the pKa shown in Table 1) in the structural moiety W of the onium salt (II) is a moiety represented by formula (II)-1.
B: The anionic moiety A (the anionic moiety having the pKa shown in Table 1) in the structural moiety W of the onium salt (II) is a moiety represented by any of formulas (II)-3 to (II)-6 and is different from the moiety represented by formula (II)-1.
C: The anionic moiety A (the anionic moiety having the pKa shown in Table 1) in the structural moiety W of the onium salt (II) is a moiety represented by formula (II)-2 and is different from the moieties represented by formulas (II)-1 and (II)-3 to (II)-6.

TABLE 5
Table 5

| Alkali | Resist | Condition | Condition | Cross-sectional |
|---|---|---|---|---|
| development | composition | 1 | 2 | rectangularity |
| Example 1-1 | Re-1 | A | B | A |
| Example 1-2 | Re-2 | A | A | S |
| Example 1-3 | Re-3 | A | C | B |
| Example 1-4 | Re-4 | A | A | S |
| Example 1-5 | Re-5 | C | B | C |
| Example 1-6 | Re-6 | B | B | B |
| Example 1-7 | Re-7 | B | A | A |
| Example 1-8 | Re-8 | A | A | S |
| Example 1-9 | Re-9 | B | A | A |
| Example 1-10 | Re-10 | B | B | B |
| Example 1-11 | Re-11 | B | B | B |
| Example 1-12 | Re-12 | B | B | B |
| Example 1-13 | Re-13 | A | B | A |
| Example 1-14 | Re-14 | B | B | B |
| Example 1-15 | Re-15 | A | B | A |
| Example 1-16 | Re-16 | B | B | B |
| Example 1-17 | Re-17 | A | B | A |
| Example 1-18 | Re-18 | A | B | A |
| Example 1-19 | Re-19 | B | B | B |
| Example 1-20 | Re-20 | C | A | B |
| Example 1-21 | Re-21 | C | B | C |
| Example 1-22 | Re-22 | A | C | B |
| Example 1-23 | Re-23 | A | B | A |
| Example 1-24 | Re-24 | B | B | B |
| Example 1-25 | Re-25 | A | B | A |
| Example 1-26 | Re-26 | A | B | A |
| Example 1-27 | Re-27 | A | A/A | S |
| Example 1-28 | Re-28 | B | B | B |
| Example 1-29 | Re-29 | B | B | B |
| Example 1-30 | Re-30 | B | B | B |
| Example 1-31 | Re-31 | C | C | D |
| Example 1-32 | Re-32 | A | A | S |
| Example 1-33 | Re-33 | A | A/A | S |
| Example 1-34 | Re-34 | B | B | B |
| Example 1-35 | Re-35 | A | A/A | S |
| Comparative | Re-C1 | D | B | E |
| Example 1-1 | | | | |
| Comparative | Re-C2 | A | — | E |
| Example 1-2 | | | | |

TABLE 6
Table 6

| Organic solvent | Resist | Condition | Condition | Cross-sectional |
|---|---|---|---|---|
| development | composition | 1 | 2 | rectangularity |
| Example 2-1 | Re-1 | A | B | A |
| Example 2-2 | Re-2 | A | A | S |
| Example 2-3 | Re-3 | A | C | B |
| Example 2-4 | Re-4 | A | A | S |
| Example 2-5 | Re-5 | C | B | C |
| Example 2-6 | Re-6 | B | B | B |
| Example 2-7 | Re-7 | B | A | A |
| Example 2-8 | Re-8 | A | A | S |
| Example 2-9 | Re-9 | B | A | A |
| Example 2-10 | Re-10 | B | B | B |
| Example 2-11 | Re-11 | B | B | B |
| Example 2-12 | Re-12 | B | B | B |
| Example 2-13 | Re-13 | A | B | A |
| Example 2-14 | Re-14 | B | B | B |
| Example 2-15 | Re-15 | A | B | A |
| Example 2-16 | Re-16 | B | B | B |
| Example 2-17 | Re-17 | A | B | A |
| Example 2-18 | Re-18 | A | B | A |
| Example 2-19 | Re-19 | B | B | B |
| Example 2-20 | Re-20 | C | A | B |
| Example 2-21 | Re-21 | C | B | C |
| Example 2-22 | Re-22 | A | C | B |
| Example 2-23 | Re-23 | A | B | A |
| Example 2-24 | Re-24 | B | B | B |
| Example 2-25 | Re-25 | A | B | A |
| Example 2-26 | Re-26 | A | B | A |
| Example 2-27 | Re-27 | A | A/A | S |
| Example 2-28 | Re-28 | B | B | B |
| Example 2-29 | Re-29 | B | B | B |
| Example 2-30 | Re-30 | B | B | B |
| Example 2-31 | Re-31 | C | C | D |
| Example 2-32 | Re-32 | A | A | S |
| Example 2-33 | Re-33 | A | A/A | S |
| Example 2-34 | Re-34 | B | B | B |
| Example 2-35 | Re-35 | A | A/A | S |
| Comparative | Re-C1 | D | B | E |
| Example 2-1 | | | | |
| Comparative | Re-C2 | A | — | E |
| Example 2-2 | | | | |

As can be seen from the results in Tables 5 and 6, the resist compositions of the invention (actinic ray-sensitive or radiation-sensitive resin compositions) have good cross-sectional rectangularity.

Comparisons of Examples 1-3, 1-22, and 1-31 and Examples 2-3, 2-22, and 2-31 with the other Examples indicate that, when the anionic moiety A in the structural moiety W was the moiety represented by any of formulas (II)-1 and (II)-3 to (II)-6, the effects of the invention were higher.

Comparisons of Examples 1-2, 1-4, 1-7 to 1-9, 1-20, 1-27, 1-32, 1-33, and 1-35 and Examples 2-2, 2-4, 2-7 to 2-9, 2-20, 2-27, 2-32, 2-33, and 2-35 with the other Examples indicate that, when the anionic moiety A in the structural moiety W was the moiety represented by formula (II)-1, the effects of the invention were higher.

Comparisons of Examples 1-1 to 1-4, 1-8, 1-13, 1-15, 1-17, 1-18, 1-22, 1-23, 1-25 to 1-27, 1-32, 1-33, and 1-35 and Examples 2-1 to 2-4, 2-8, 2-13, 2-15, 2-17, 2-18, 2-22, 2-23, 2-25 to 2-27, 2-32, 2-33, and 2-35 with the other Examples indicate that, when n in formula (1) was 2 to 5, the effects of the invention were higher.

Comparisons of Examples 1-5, 1-20, 1-21, and 1-31 and Examples 2-5, 2-20, 2-21, and 2-31 with the other Examples indicate that, when R_{S} in formula (1) represents —CO—OR_{S1}, —O—CO—R_{S1}, —O—CO—O—R_{S1}, —SO₂—R_{S1}, or —SO₃—R_{S1} and R_{S1} represents a monovalent substituent, the effects of the invention were higher.

Comparisons of Examples 1-2, 1-4, 1-8, 1-27, 1-32, 1-33, and 1-35 and Example 2-2, 2-4, 2-8, 2-27, 2-32, 2-33, and 2-35 with the other Examples indicate that, when R_{S} in formula (1) represents —CO—OR_{S1} or —O—CO—R_{S1} and R_{S1} represents a monovalent substituent, the effects of the invention were higher.

What is claimed is:

1. An actinic ray-sensitive or radiation-sensitive resin composition comprising:
a resin having a group that is decomposed by the action of an acid to generate a polar group;
an onium salt (I) that generates an acid represented by formula (1) upon irradiation with actinic rays or radiation; and
an onium salt (II) having at least one structural moiety W that includes an anionic moiety A and a cationic moiety M and forms an acidic moiety represented by HA upon irradiation with the actinic rays or radiation,
wherein the acid dissociation constant derived from the acidic moiety that is represented by HA and that is formed by replacing the cationic moiety in the structural moiety W with H⁺ is larger than the acid dissociation constant of the acid represented by formula (1): [symbol]
wherein, in formula (1), X represents —OH or —NH—SO₂—R_{X}; R_{X} represents an alkyl group having at least one fluorine atom;
Rf represents a fluorine atom or an alkyl group having at least one fluorine atom; Y represents a single bond, an oxygen atom, or a sulfur atom;
Ar represents an n+1 valent aromatic ring group optionally having a substituent other than R_{S};
R_{S} or R_{S}'s each independently represent —O—R_{S1}, —CO—R_{S1}, —CO—O—R_{S1}, —O—CO—R_{S1}, —O—CO—O—R_{S1}, —SO₂-R_{S1}, or —SO₃-R_{S1}; R_{S1} represents a monovalent substituent;
m represents an integer of 1 or more; and
n represents an integer of 1 to 5.

2. The actinic ray-sensitive or radiation-sensitive resin composition according to claim 1, wherein the anionic moiety A in the structural moiety W is a moiety represented by any of formulas (II)-1 to (II)-6: [symbol]
wherein, in formulas (II)-1 to (II)-6, * represents a direct bond.

3. The actinic ray-sensitive or radiation-sensitive resin composition according to claim 2, wherein the anionic moiety A in the structural moiety W is the moiety represented by any of formulas (II)-1 and (II)-3 to (II)-6.

4. The actinic ray-sensitive or radiation-sensitive resin composition according to claim 3, wherein the anionic moiety A in the structural moiety W is the moiety represented by formula (II)-1.

5. The actinic ray-sensitive or radiation-sensitive resin composition according to claim 1, wherein n in formula (1) is 2 to 5.

6. The actinic ray-sensitive or radiation-sensitive resin composition according to claim 1, wherein R_{S} or R_{S}'s in formula (1) each independently represent —CO—OR_{S1}, —O—CO—R_{S1}, —O—CO—O—R_{S1}, —SO₂-R_{S1}, or —SO₃-R_{S1}, and R_{S1} represents a monovalent substituent.

7. The actinic ray-sensitive or radiation-sensitive resin composition according to claim 1, wherein R_{S} or R_{S}'s in formula (1) each independently represent —CO—OR_{S1} or —O—CO—R_{S1}, and R_{S1} represents a monovalent substituent.

8. A resist film formed using the actinic ray-sensitive or radiation-sensitive resin composition according to claim 1.

9. A pattern forming method comprising:
a resist film forming step of forming a resist film on a substrate using the actinic ray-sensitive or radiation-sensitive resin composition according to claim 1;
an exposure step of exposing the resist film to light; and
a developing step of developing the exposed resist film using a developer.

10. The pattern forming method according to claim 9, wherein, in the exposure step, EUV light is used for exposure.

11. The pattern forming method according to claim 9, wherein the developer includes an organic solvent.

12. The pattern forming method according to claim 11, wherein the organic solvent includes butyl acetate.

13. A method for manufacturing an electronic device, the method comprising the pattern forming method according to claim 9.

14. The actinic ray-sensitive or radiation-sensitive resin composition according to claim 2, wherein n in formula (1) is 2 to 5.

15. The actinic ray-sensitive or radiation-sensitive resin composition according to claim 3, wherein n in formula (1) is 2 to 5.

16. The actinic ray-sensitive or radiation-sensitive resin composition according to claim 4, wherein n in formula (1) is 2 to 5.

17. The actinic ray-sensitive or radiation-sensitive resin composition according to claim 2, wherein R_{S} or R_{S}'s in formula (1) each independently represent —CO—OR_{S1}, —O—CO—R_{S1}, —O—CO—O—R_{S1}, —SO₂-R_{S1}, or —SO₃-R_{S1}, and R_{S1} represents a monovalent substituent.

18. The actinic ray-sensitive or radiation-sensitive resin composition according to claim 3, wherein R_{S} or R_{S}'s in formula (1) each independently represent —CO—OR_{S1}, —O—CO—R_{S1}, —O—CO—O—R_{S1}, —SO₂-R_{S1}, or —SO₃-R_{S1}, and R_{S1} represents a monovalent substituent.

19. The actinic ray-sensitive or radiation-sensitive resin composition according to claim 4, wherein R_{S} or R_{S}'s in formula (1) each independently represent —CO—OR_{S1}, —O—CO—R_{S1}, —O—CO—O—R_{S1}, —SO₂-R_{S1}, or —SO₃-R_{S1}, and R_{S1} represents a monovalent substituent.

20. The actinic ray-sensitive or radiation-sensitive resin composition according to claim 2, wherein R_{S} or R_{S}'s in formula (1) each independently represent —CO—OR_{S1} or —O—CO—R_{S1}, and R_{S1} represents a monovalent substituent.
